(19) United States
(12) Patent Application Publication
(10) Pub. No.: US 2024/0301284 A1
(43) Pub. Date: Sep. 12, 2024

(54) ORGANIC COMPOUND AND ELECTROLUMINESCENT DEVICE

(71) Applicant: SICHUAN AG-RAY NEW MATERIALS CO., LTD, Meishan City (CN)

(72) Inventors: Liangliang YAN, Foshan (CN); Shaofu CHEN, Foshan (CN); Lei DAI, Foshan (CN); Lifei CAI, Foshan (CN)

(73) Assignee: SICHUAN AG-RAY NEW MATERIALS CO., LTD, Meishan City (CN)

(21) Appl. No.: 18/658,193

(22) Filed: May 8, 2024

Related U.S. Application Data
(63) Continuation of application No. PCT/CN2022/123697, filed on Oct. 4, 2022.

(30) Foreign Application Priority Data
Nov. 9, 2021 (CN) 202111320218.1
Sep. 26, 2022 (CN) 202211173070.8

(51) Int. Cl. C09K 11/06; H10K 50/11; H10K 50/15; H10K 85/60

(52) U.S. Cl. CPC C09K 11/06; H10K 50/11; H10K 50/15; C09K 2211/1011; H10K 85/60

(57) ABSTRACT

The present disclosure relates to an organic compound and an electroluminescent device. The organic compound has the structure as shown in formula (1), which can be used in an organic electroluminescent device as a hole injection layer material.

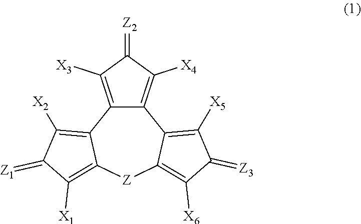

### CROSS-REFERENCE TO RELATED APPLICATIONS

This application is a bypass continuation of International Application No.: PCT/CN2022/123697 filed on Oct. 4, 2022, which claims the benefit of Chinese Patent Application No. 202111320218.1, filed on Nov. 9, 2021, and the Chinese Patent Application No. 202211173070.8, filed on Sep. 26, 2022, the entire contents of which are incorporated by reference in their entirety.

### TECHNICAL FIELD

The present disclosure relates to the technical field of organic electroluminescence, especially to an organic luminescent material, in particular to an organic compound and use thereof in an organic electroluminescent device.

### BACKGROUND

At present, as a new generation of display technology, organic electroluminescent devices (OLEDs) have attracted more and more attention in both display and lighting technologies, and their application prospects are very broad. However, with respect to market application requirements, the performance of OLED devices in terms of luminous efficiency, driving voltage, service life, etc., still needs to be further strengthened and improved.

Generally, the basic structure of OLED devices is that various organic functional material thin films with different functions are sandwiched between metal electrodes, just like a sandwich structure. Driven by current, holes and electrons are injected from an anode and a cathode, respectively. After moving for a certain distance, the holes and electrons are recombined in a luminescent layer and released in the form of light or heat, thus causing the OLEDs to emit light. However, organic functional materials are the core components of organic electroluminescent devices, and their thermal stability, photochemical stability, electrochemical stability, quantum yield, film-forming stability, crystallinity, color saturation, etc., are all main factors that affect the performance of the devices.

Generally, in organic light-emitting display devices, a hole injection layer will be introduced. The function of this layer of material is mainly to improve some defects of anode ITO and facilitate injection of holes into the device from ITO to reduce the driving voltage of the device and improve the stability of the device. At present, there are two main technologies to use a hole injection layer, one of which is to use a single material such as HATCN, F4-TCNQ, F6TCNNQ, CuPc, etc. Usually, this type of material has a relatively deep LUMO energy level. However, this type of material causes substantial lateral crosstalk, and in batch use, device reproducibility and stability problems caused by the film-forming properties and crystallinity of the material and other reasons need to be improved. The second one of them is a technical solution in which a material with relatively deep LUMO is used as a P-type dopant and a hole transport material with matched HOMO energy level is used as a matrix, mainly including NDP-9. However, the LUMO energy level of this type of material still needs to be further improved to reduce the device voltage, and there is also the problem of lateral crosstalk, which needs to be improved.

At present, various compounds with relatively deep LUMO have been developed as P-type dopants. For example, China patent document CN 101330129 B discloses a class of oxocarbon, pseudooxocarbon and radialene compounds that are applied to OLED devices as hole injection layers in a P-type dopant mode; China patent document CN 102439746 B discloses a class of OLED devices comprising compound

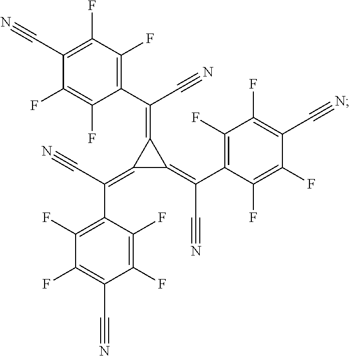 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

China patent CN 109422666 A discloses a class of OLED devices comprising compounds based on truxene

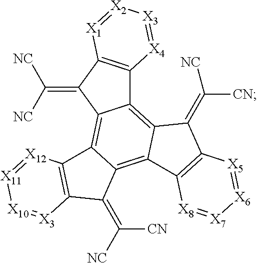

China patent CN 111454276 A discloses a class of structures having quinone fused to two five-membered heterocyclic rings as p-type dopants; in China patents CN 112745333 A, CN 109912619, CN 113087711 A, and CN 113321620A, structures such as dehydrobenzoxazole, dehydrobenzodithiazole or dehydrobenzodiselenazole are mainly used as P-type dopants; China patent CN 109928894 A discloses a class of radialene compounds that are applied to OLED devices as hole injection layers in a P-type dopant mode; China patent CN 109836436 B discloses a class of dithiophene structures as P-type dopants; China patent CN 110437103 B discloses a class of cyclic structures as P-type dopants; China patent CN 110483529 B discloses a class of fused ring structures as P-type dopants, and particularly discloses compound

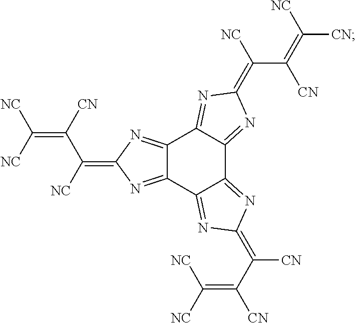 [SMILES: N#CC(C#N)=C(C#N)C(C#N)=C1N=c2c3c(c4c(c2=N1)=NC(=C(C#N)C(C#N)=C(C#N)C#N)N=4)=NC(=C(C#N)C(C#N)=C(C#N)C#N)N=3]

and China patent CN 110938085 B discloses a class of radialene structures as P-type dopants. However, the device performance of the above-mentioned materials exhibits certain good results in terms of LUMO energy level reduction and device performance improvement. However, it is particularly important and urgent to develop a high-performance hole injection layer material in order to meet the increasing requirements of device performance, particularly voltage, efficiency and lifetime, and to improve the lateral crosstalk between red, green and blue.

### SUMMARY

The present disclosure has been completed in order to solve the above-mentioned task and aims to provide a high-performance organic electroluminescent device and a novel material that can realize such an organic electroluminescent device.

In order to achieve the above-mentioned objective, the present inventors have repeatedly conducted in-depth studies, and as a result, found that high-performance organic electroluminescent devices can be obtained by using an organic compound shown in the following formula (1).

The organic compound has a structure shown in formula (1) The compound provided by the present disclosure has the advantages of not only a relatively deep LUMO energy level and a low sublimation temperature but also a good optical and electrical stability, a low driving voltage, a long device lifetime, a low transverse conductivity, etc., which can be used in organic light-emitting devices, particularly as a hole injection layer, and has the potential to be applied to the Active-matrix organic light-emitting diode (AMOLED) industry.

An organic compound having a structure shown in formula (1) is provided,

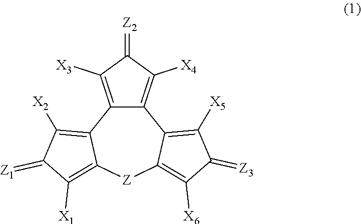

wherein Z is a single bond, O, S, SO, or SO₂;
X₁-X₆ are each independently X, X-substituted C6-C30 aryl or X-substituted C2-C30 heteroaryl, and X is F, CN, or completely or partially fluorine-substituted C1-C4 fluoroalkyl;
Z₁-Z₃ are each independently O, S, Se, NR₁, or CR₂R₃;
wherein R₁-R₃ are each independently selected from the group consisting of hydrogen, deuterium, halogen, CN, substituted or unsubstituted C1-C20 alkyl, substituted or unsubstituted C3-C20 cycloalkyl, substituted or unsubstituted C1-C20 heteroalkyl, substituted or unsubstituted C7-C30 aralkyl, substituted or unsubstituted C1-C20 alkoxy, substituted or unsubstituted C6-C30 aryloxy, substituted or unsubstituted C2-C20 alkenyl, substituted or unsubstituted C2-C20 alkynyl, substituted or unsubstituted C6-C30 aryl, substituted or unsubstituted C3-C30 heteroaryl, substituted or unsubstituted C3-C20 alkylsilyl, substituted or unsubstituted C6-C20 arylsilyl, substituted or unsubstituted C1-C6 alkylsulfinyl, substituted or unsubstituted C1-C6 alkylsulfonyl, and substituted or unsubstituted C1-C6 alkylcarbonyl;
wherein at least one of R₁-R₃ is a group having an electron-withdrawing group;
wherein the substituents on R₁-R₃ are F, CN, completely or partially fluorine-substituted C1-C4 fluoroalkyl, completely or partially fluorine-substituted C1-C4 alkylsulfinyl, completely or partially fluorine-substituted C1-C4 alkylsulfonyl, or completely or partially fluorine-substituted C1-C4 alkylcarbonyl; and
wherein the heteroalkyl and heteroaryl contain at least one O, N or S heteroatom.

In an embodiment, Z is a single bond, O, or SO.

In an embodiment, Z is a single bond and has a structure shown in formula (2),

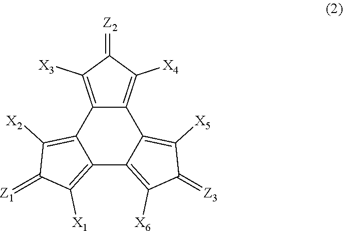

wherein
X₁-X₆ are each independently X, X-substituted C6-C20 aryl or X-substituted C2-C10 heteroaryl, and X is F, CN, or completely or partially fluorine-substituted C1-C4 fluoroalkyl; and
Z₁-Z₃ are each independently O, S, NR₁, or CR₂R₃;
wherein R₁-R₃ are each independently selected from the group consisting of hydrogen, deuterium, halogen, CN, substituted or unsubstituted C1-C10 alkyl, substituted or unsubstituted C3-C10 cycloalkyl, substituted or unsubstituted C1-C10 heteroalkyl, substituted or unsubstituted C7-C20 aralkyl, substituted or unsubstituted C6-C20 aryl, substituted or unsubstituted C3-C20 heteroaryl, substituted or unsubstituted C1-C4 alkylsulfinyl, substituted or unsubstituted C1-C4 alkylsulfonyl, and substituted or unsubstituted C1-C4 alkylcarbonyl.

In an embodiment, at least two of Z₁-Z₃ are CR₂R₃.

In an embodiment, Z₁-Z₃ are all CR₂R₃.

In an embodiment, among these CR₂R₃, at least two are groups having an electron-withdrawing group.

In an embodiment, R₂ and R₃ are both groups having electron-withdrawing property.

R₂-R₃ are each independently selected from the group consisting of halogen, CN, substituted or unsubstituted C1-C6 alkyl, substituted or unsubstituted C7-C10 aralkyl, substituted or unsubstituted C6-C12 aryl, substituted or unsubstituted C3-12 heteroaryl, substituted or unsubstituted C1-C4 alkylsulfinyl, substituted or unsubstituted C1-C4 alkylsulfonyl, and substituted or unsubstituted C1-C4 alkylcarbonyl.

The substituents on R₂-R₃ are F, CN, CF₃, CF₃-sulfinyl, or CF₃-sulfonyl;
wherein X₁-X₆ are each independently X.

In an embodiment, X1-X6 are F, CN, or CF₃.

In an embodiment, the group having electron-withdrawing property is F, CN, CF₃, pyridine, pyrimidine, pyridazine, pyrazine, thiazole, oxazole, triazine, sulfinyl, sulfonyl, carbonyl, or C6-C12 aryl or C3-C12 heteroaryl containing one of the foregoing groups having electron-withdrawing property.

In an embodiment, formula (1) is one of the following structural formulas:

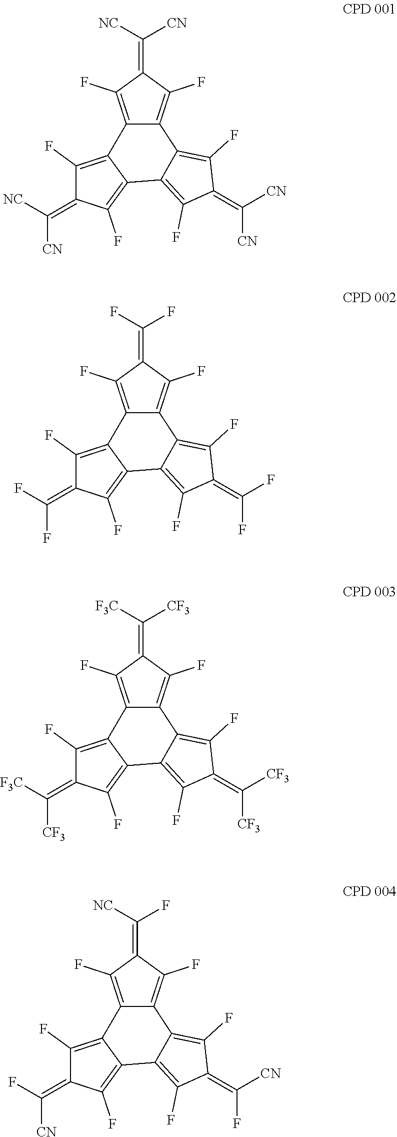 [SMILES: N#CC(C#N)=C1C(F)=c2c3c(c4c(c2=C1F)=C(F)C(=C(C#N)C#N)C=4F)=C(F)C(=C(C#N)C#N)C=3F ; FC(F)=C1C(F)=c2c3c(c4c(c2=C1F)=C(F)C(=C(F)F)C=4F)=C(F)C(=C(F)F)C=3F ; FC1=c2c3c(c4c(c2=C(F)C1=C(C(F)(F)F)C(F)(F)F)=C(F)C(=C(C(F)(F)F)C(F)(F)F)C=4F)=C(F)C(=C(C(F)(F)F)C(F)(F)F)C=3F ; N#CC(F)=C1C(F)=c2c3c(c4c(c2=C1F)=C(F)C(=C(F)C#N)C=4F)=C(F)C(=C(F)C#N)C=3F]

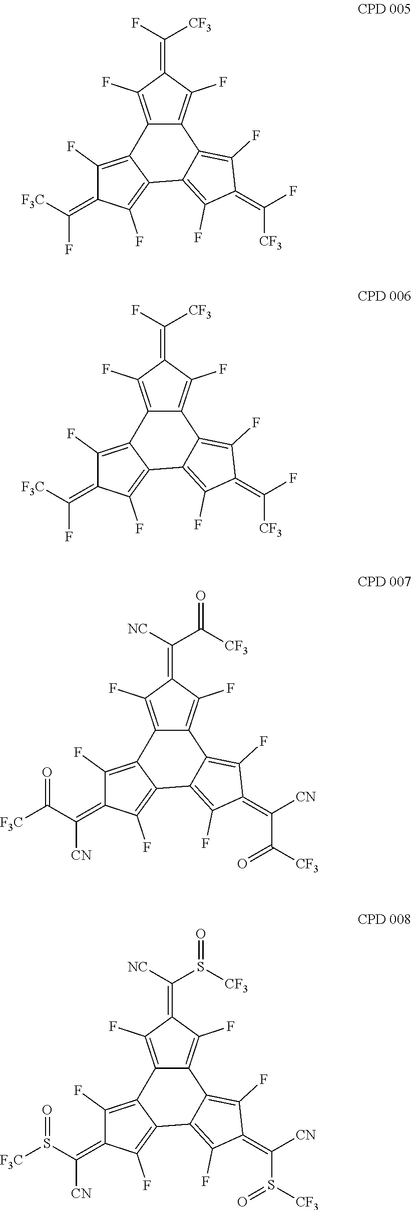 [SMILES: FC(=C1C(F)=c2c3c(c4c(c2=C1F)=C(F)C(=C(F)C(F)(F)F)C=4F)=C(F)C(=C(F)C(F)(F)F)C=3F)C(F)(F)F ; FC(=C1C(F)=c2c3c(c4c(c2=C1F)=C(F)C(=C(F)C(F)(F)F)C=4F)=C(F)C(=C(F)C(F)(F)F)C=3F)C(F)(F)F ; N#CC(C(=O)C(F)(F)F)=C1C(F)=c2c3c(c4c(c2=C1F)=C(F)C(=C(C#N)C(=O)C(F)(F)F)C=4F)=C(F)C(=C(C#N)C(=O)C(F)(F)F)C=3F ; N#CC(=C1C(F)=c2c3c(c4c(c2=C1F)=C(F)C(=C(C#N)S(=O)C(F)(F)F)C=4F)=C(F)C(=C(C#N)S(=O)C(F)(F)F)C=3F)S(=O)C(F)(F)F]

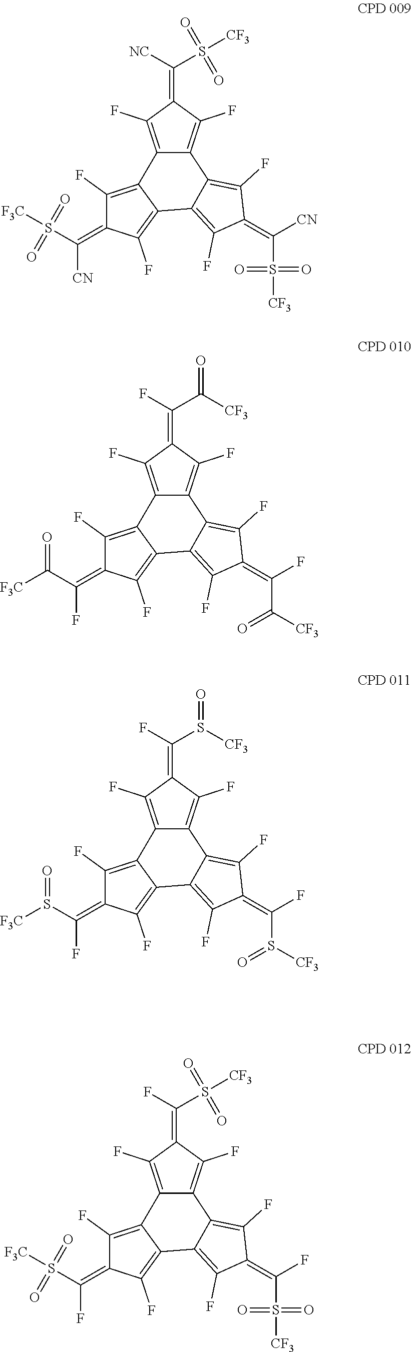 [SMILES: N#CC(=C1C(F)=c2c3c(c4c(c2=C1F)=C(F)C(=C(C#N)S(=O)(=O)C(F)(F)F)C=4F)=C(F)C(=C(C#N)S(=O)(=O)C(F)(F)F)C=3F)S(=O)(=O)C(F)(F)F ; O=C(C(F)=C1C(F)=c2c3c(c4c(c2=C1F)=C(F)C(=C(F)C(=O)C(F)(F)F)C=4F)=C(F)C(=C(F)C(=O)C(F)(F)F)C=3F)C(F)(F)F ; O=S(C(F)=C1C(F)=c2c3c(c4c(c2=C1F)=C(F)C(=C(F)S(=O)C(F)(F)F)C=4F)=C(F)C(=C(F)S(=O)C(F)(F)F)C=3F)C(F)(F)F ; O=S(=O)(C(F)=C1C(F)=c2c3c(c4c(c2=C1F)=C(F)C(=C(F)S(=O)(=O)C(F)(F)F)C=4F)=C(F)C(=C(F)S(=O)(=O)C(F)(F)F)C=3F)C(F)(F)F]

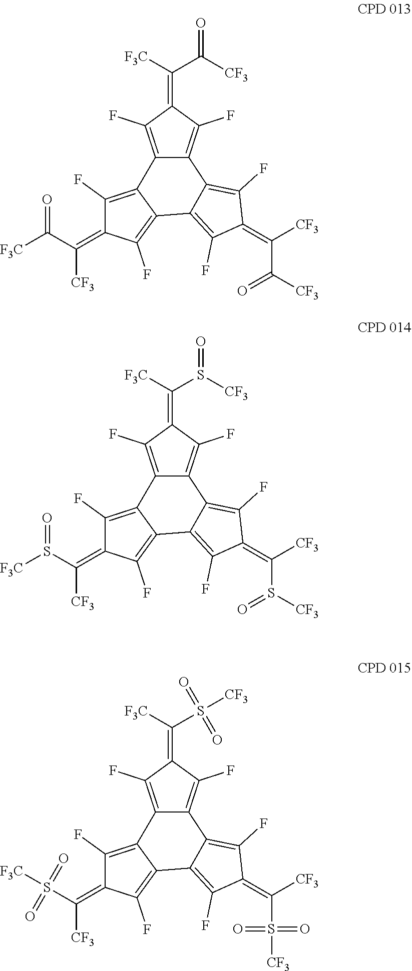 [SMILES: O=C(C(=C1C(F)=c2c3c(c4c(c2=C1F)=C(F)C(=C(C(=O)C(F)(F)F)C(F)(F)F)C=4F)=C(F)C(=C(C(=O)C(F)(F)F)C(F)(F)F)C=3F)C(F)(F)F)C(F)(F)F ; O=S(C(=C1C(F)=c2c3c(c4c(c2=C1F)=C(F)C(=C(S(=O)C(F)(F)F)C(F)(F)F)C=4F)=C(F)C(=C(S(=O)C(F)(F)F)C(F)(F)F)C=3F)C(F)(F)F)C(F)(F)F ; O=S(=O)(C(=C1C(F)=c2c3c(c4c(c2=C1F)=C(F)C(=C(C(F)(F)F)S(=O)(=O)C(F)(F)F)C=4F)=C(F)C(=C(C(F)(F)F)S(=O)(=O)C(F)(F)F)C=3F)C(F)(F)F)C(F)(F)F]

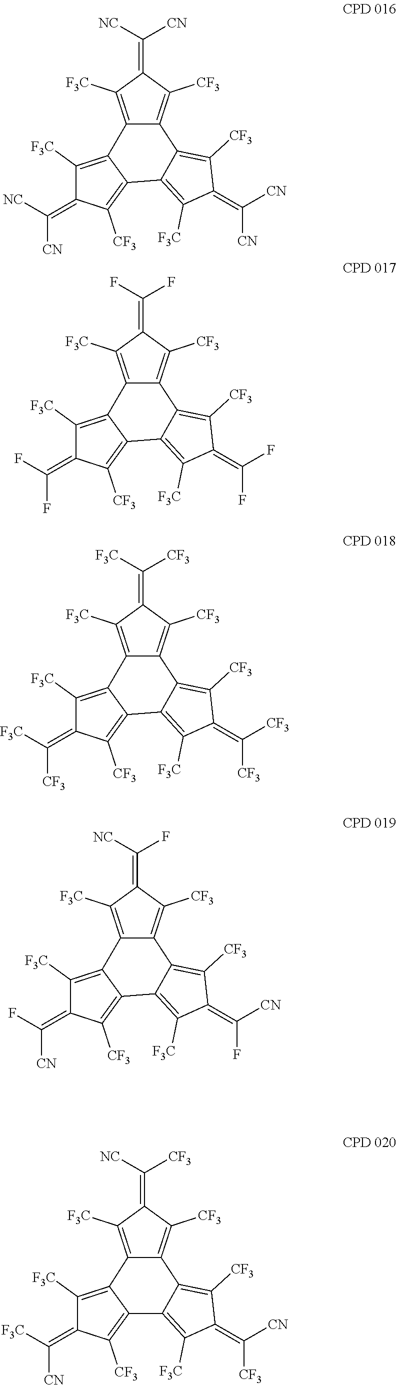

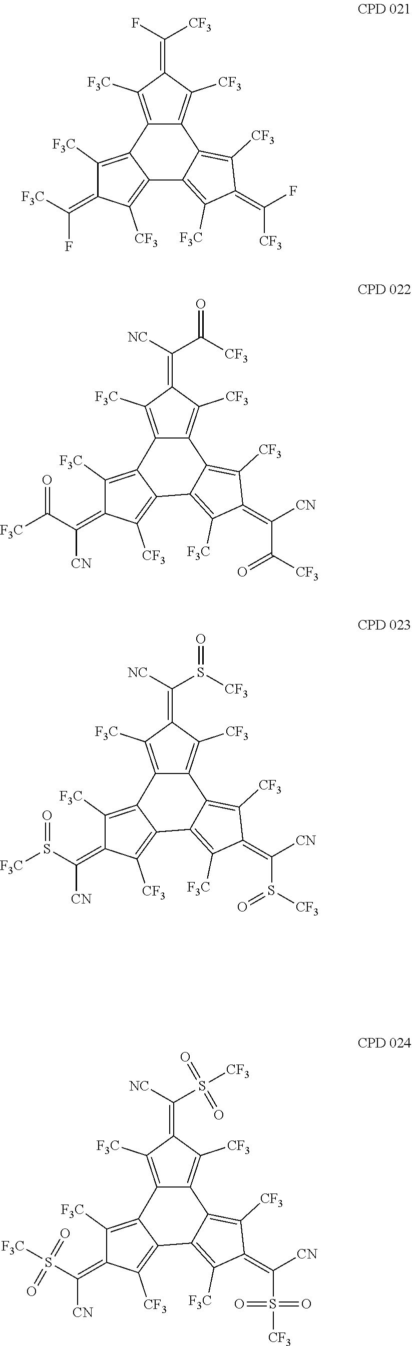

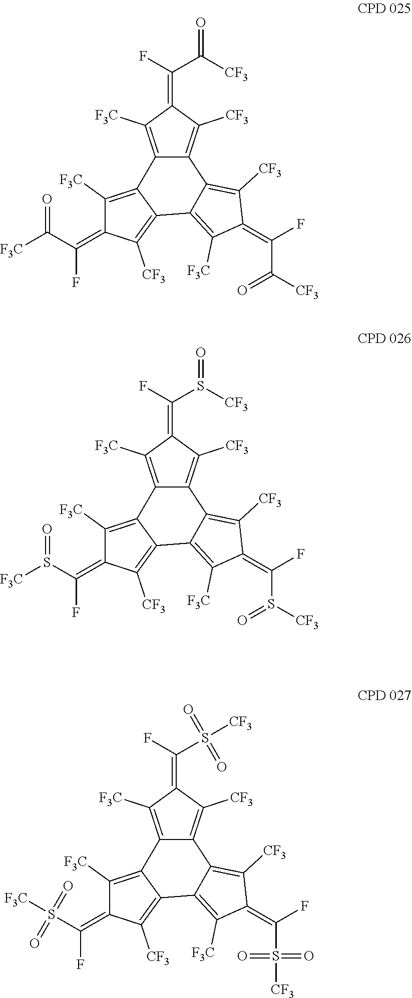 [SMILES: O=C(C(F)=C1C(C(F)(F)F)=c2c3c(c4c(c2=C1C(F)(F)F)=C(C(F)(F)F)C(=C(F)C(=O)C(F)(F)F)C=4C(F)(F)F)=C(C(F)(F)F)C(=C(F)C(=O)C(F)(F)F)C=3C(F)(F)F)C(F)(F)F ; O=S(C(F)=C1C(C(F)(F)F)=c2c3c(c4c(c2=C1C(F)(F)F)=C(C(F)(F)F)C(=C(F)S(=O)C(F)(F)F)C=4C(F)(F)F)=C(C(F)(F)F)C(=C(F)S(=O)C(F)(F)F)C=3C(F)(F)F)C(F)(F)F ; O=S(=O)(C(F)=C1C(C(F)(F)F)=c2c3c(c4c(c2=C1C(F)(F)F)=C(C(F)(F)F)C(=C(F)S(=O)(=O)C(F)(F)F)C=4C(F)(F)F)=C(C(F)(F)F)C(=C(F)S(=O)(=O)C(F)(F)F)C=3C(F)(F)F)C(F)(F)F]

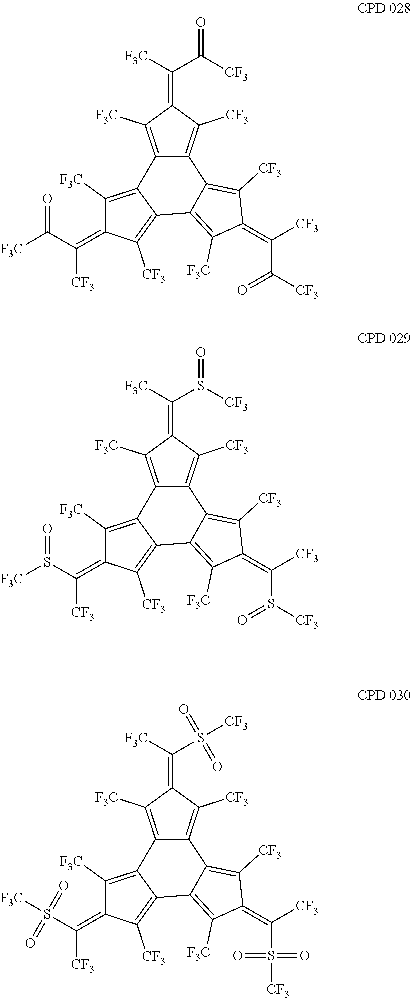

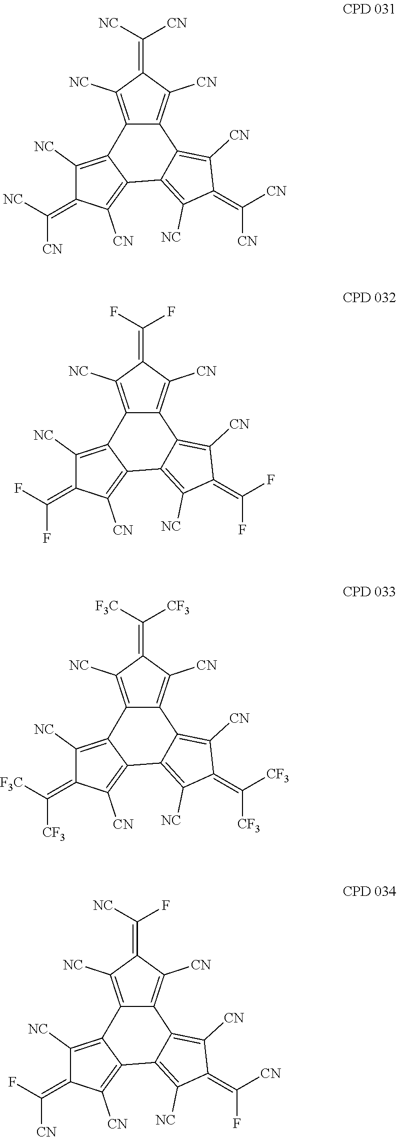 [SMILES: N#CC(C#N)=C1C(C#N)=c2c3c(c4c(c2=C1C#N)=C(C#N)C(=C(C#N)C#N)C=4C#N)=C(C#N)C(=C(C#N)C#N)C=3C#N ; N#CC1=c2c3c(c4c(c2=C(C#N)C1=C(F)F)=C(C#N)C(=C(F)F)C=4C#N)=C(C#N)C(=C(F)F)C=3C#N ; N#CC1=c2c3c(c4c(c2=C(C#N)C1=C(C(F)(F)F)C(F)(F)F)=C(C#N)C(=C(C(F)(F)F)C(F)(F)F)C=4C#N)=C(C#N)C(=C(C(F)(F)F)C(F)(F)F)C=3C#N ; N#CC(F)=C1C(C#N)=c2c3c(c4c(c2=C1C#N)=C(C#N)C(=C(F)C#N)C=4C#N)=C(C#N)C(=C(F)C#N)C=3C#N]

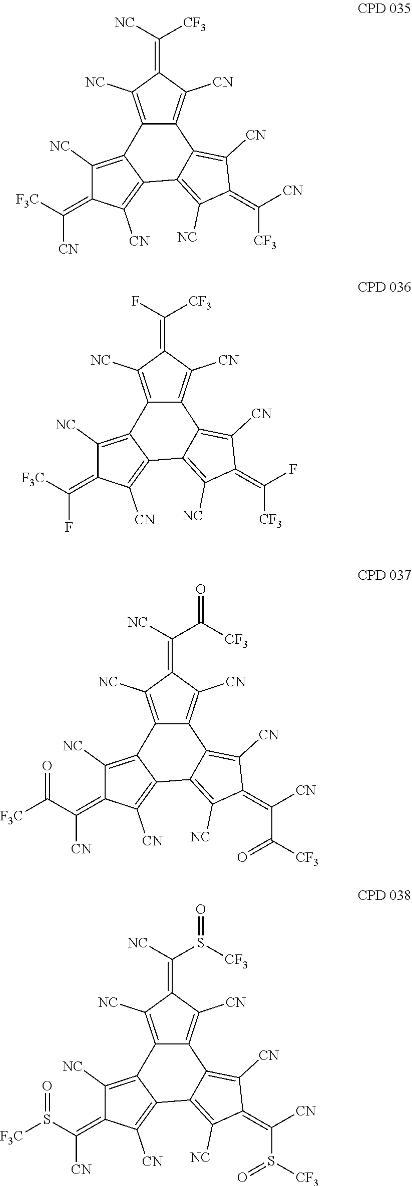 [SMILES: N#CC(=C1C(C#N)=c2c3c(c4c(c2=C1C#N)=C(C#N)C(=C(C#N)C(F)(F)F)C=4C#N)=C(C#N)C(=C(C#N)C(F)(F)F)C=3C#N)C(F)(F)F ; N#CC1=c2c3c(c4c(c2=C(C#N)C1=C(F)C(F)(F)F)=C(C#N)C(=C(F)C(F)(F)F)C=4C#N)=C(C#N)C(=C(F)C(F)(F)F)C=3C#N ; N#CC(C(=O)C(F)(F)F)=C1C(C#N)=c2c3c(c4c(c2=C1C#N)=C(C#N)C(=C(C#N)C(=O)C(F)(F)F)C=4C#N)=C(C#N)C(=C(C#N)C(=O)C(F)(F)F)C=3C#N ; N#CC(=C1C(C#N)=c2c3c(c4c(c2=C1C#N)=C(C#N)C(=C(C#N)S(=O)C(F)(F)F)C=4C#N)=C(C#N)C(=C(C#N)S(=O)C(F)(F)F)C=3C#N)S(=O)C(F)(F)F]

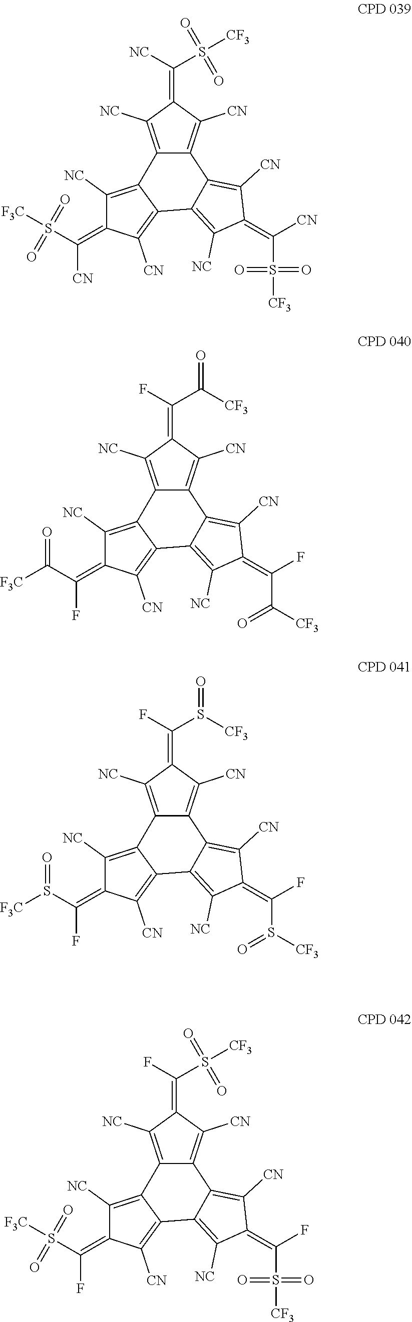 [SMILES: N#CC(=C1C(C#N)=c2c3c(c4c(c2=C1C#N)=C(C#N)C(=C(C#N)S(=O)(=O)C(F)(F)F)C=4C#N)=C(C#N)C(=C(C#N)S(=O)(=O)C(F)(F)F)C=3C#N)S(=O)(=O)C(F)(F)F ; N#CC1=c2c3c(c4c(c2=C(C#N)C1=C(F)C(=O)C(F)(F)F)=C(C#N)C(=C(F)C(=O)C(F)(F)F)C=4C#N)=C(C#N)C(=C(F)C(=O)C(F)(F)F)C=3C#N ; N#CC1=c2c3c(c4c(c2=C(C#N)C1=C(F)S(=O)C(F)(F)F)=C(C#N)C(=C(F)S(=O)C(F)(F)F)C=4C#N)=C(C#N)C(=C(F)S(=O)C(F)(F)F)C=3C#N ; N#CC1=c2c3c(c4c(c2=C(C#N)C1=C(F)S(=O)(=O)C(F)(F)F)=C(C#N)C(=C(F)S(=O)(=O)C(F)(F)F)C=4C#N)=C(C#N)C(=C(F)S(=O)(=O)C(F)(F)F)C=3C#N]

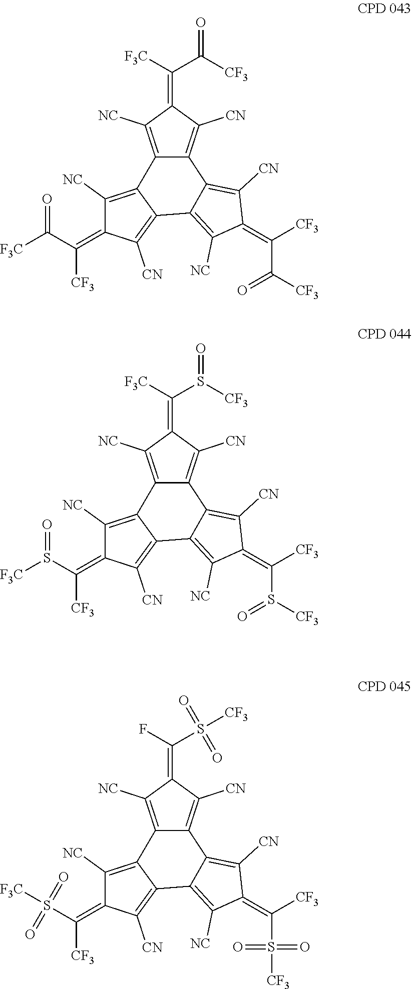 [SMILES: N#CC1=c2c3c(c4c(c2=C(C#N)C1=C(C(=O)C(F)(F)F)C(F)(F)F)=C(C#N)C(=C(C(=O)C(F)(F)F)C(F)(F)F)C=4C#N)=C(C#N)C(=C(C(=O)C(F)(F)F)C(F)(F)F)C=3C#N ; N#CC1=c2c3c(c4c(c2=C(C#N)C1=C(S(=O)C(F)(F)F)C(F)(F)F)=C(C#N)C(=C(S(=O)C(F)(F)F)C(F)(F)F)C=4C#N)=C(C#N)C(=C(S(=O)C(F)(F)F)C(F)(F)F)C=3C#N ; N#CC1=c2c(c3c(c4c2=C(C#N)/C(=C(\F)S(=O)(=O)C(F)(F)F)C=4C#N)=C(C#N)/C(=C(\C(F)(F)F)S(=O)(=O)C(F)(F)F)C=3C#N)=C(C#N)/C1=C(\C(F)(F)F)S(=O)(=O)C(F)(F)F]

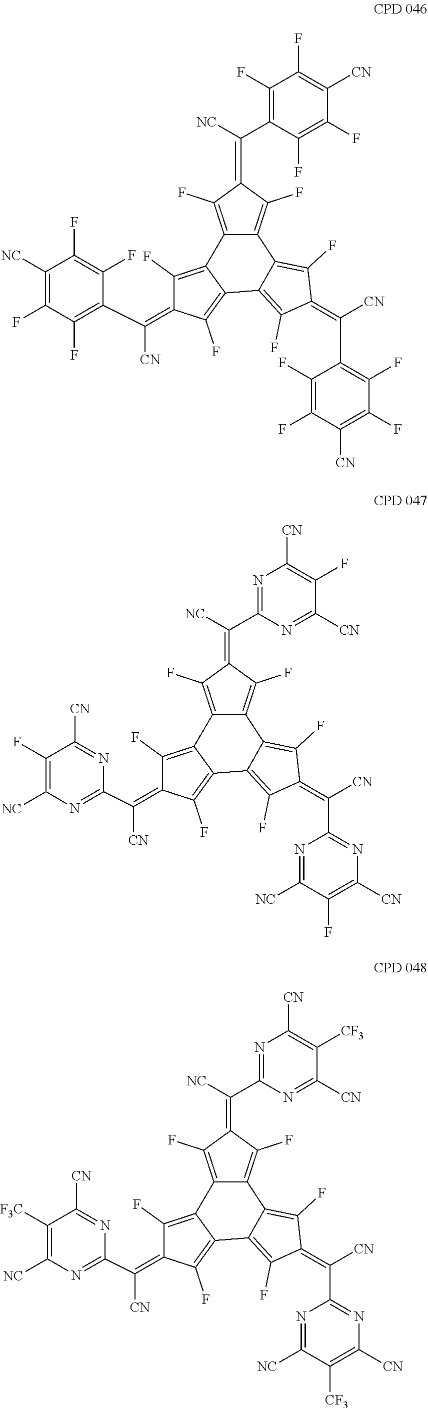 [SMILES: N#CC(=C1C(F)=c2c3c(c4c(c2=C1F)=C(F)C(=C(C#N)c1c(F)c(F)c(C#N)c(F)c1F)C=4F)=C(F)C(=C(C#N)c1c(F)c(F)c(C#N)c(F)c1F)C=3F)c1c(F)c(F)c(C#N)c(F)c1F ; N#CC(=C1C(F)=c2c3c(c4c(c2=C1F)=C(F)C(=C(C#N)c1nc(C#N)c(F)c(C#N)n1)C=4F)=C(F)C(=C(C#N)c1nc(C#N)c(F)c(C#N)n1)C=3F)c1nc(C#N)c(F)c(C#N)n1 ; N#CC(=C1C(F)=c2c3c(c4c(c2=C1F)=C(F)C(=C(C#N)c1nc(C#N)c(C(F)(F)F)c(C#N)n1)C=4F)=C(F)C(=C(C#N)c1nc(C#N)c(C(F)(F)F)c(C#N)n1)C=3F)c1nc(C#N)c(C(F)(F)F)c(C#N)n1]

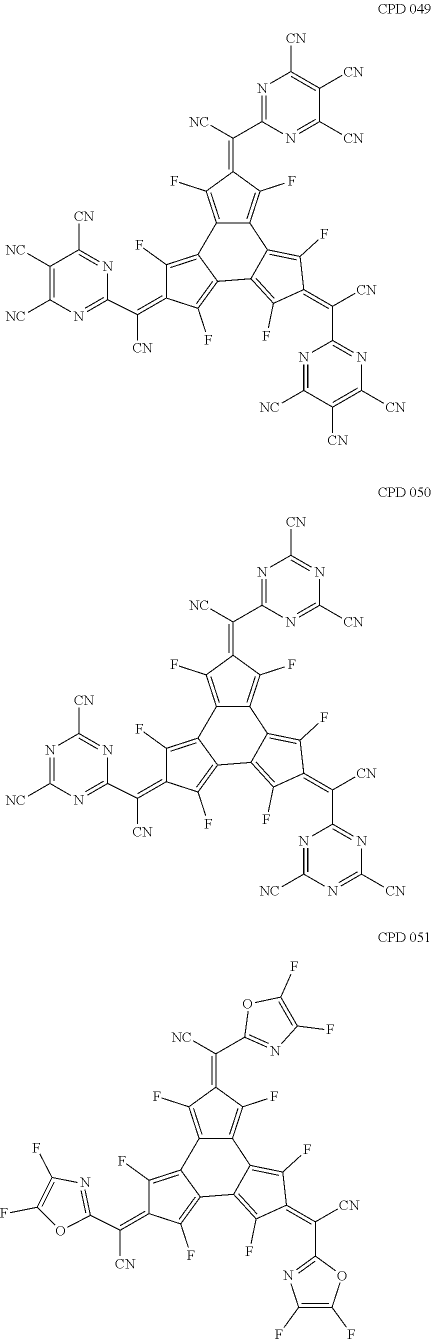 [SMILES: N#CC(=C1C(F)=c2c3c(c4c(c2=C1F)=C(F)C(=C(C#N)c1nc(C#N)c(C#N)c(C#N)n1)C=4F)=C(F)C(=C(C#N)c1nc(C#N)c(C#N)c(C#N)n1)C=3F)c1nc(C#N)c(C#N)c(C#N)n1 ; N#CC(=C1C(F)=c2c3c(c4c(c2=C1F)=C(F)C(=C(C#N)c1nc(C#N)nc(C#N)n1)C=4F)=C(F)C(=C(C#N)c1nc(C#N)nc(C#N)n1)C=3F)c1nc(C#N)nc(C#N)n1 ; N#CC(=C1C(F)=c2c3c(c4c(c2=C1F)=C(F)C(=C(C#N)c1nc(F)c(F)o1)C=4F)=C(F)C(=C(C#N)c1nc(F)c(F)o1)C=3F)c1nc(F)c(F)o1]

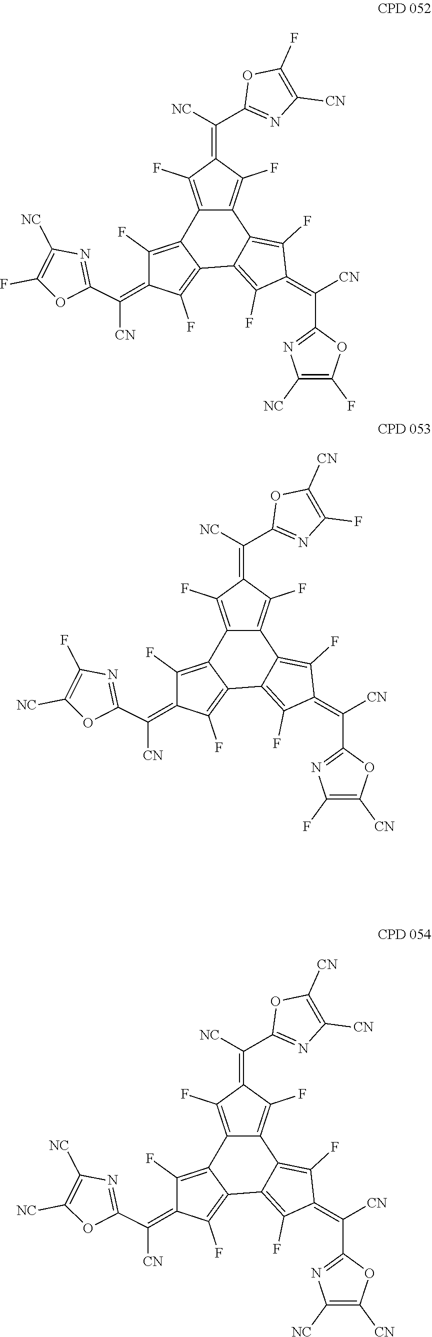 [SMILES: N#CC(=C1C(F)=c2c3c(c4c(c2=C1F)=C(F)C(=C(C#N)c1nc(C#N)c(F)o1)C=4F)=C(F)C(=C(C#N)c1nc(C#N)c(F)o1)C=3F)c1nc(C#N)c(F)o1 ; N#CC(=C1C(F)=c2c3c(c4c(c2=C1F)=C(F)C(=C(C#N)c1nc(F)c(C#N)o1)C=4F)=C(F)C(=C(C#N)c1nc(F)c(C#N)o1)C=3F)c1nc(F)c(C#N)o1 ; N#CC(=C1C(F)=c2c3c(c4c(c2=C1F)=C(F)C(=C(C#N)c1nc(C#N)c(C#N)o1)C=4F)=C(F)C(=C(C#N)c1nc(C#N)c(C#N)o1)C=3F)c1nc(C#N)c(C#N)o1]

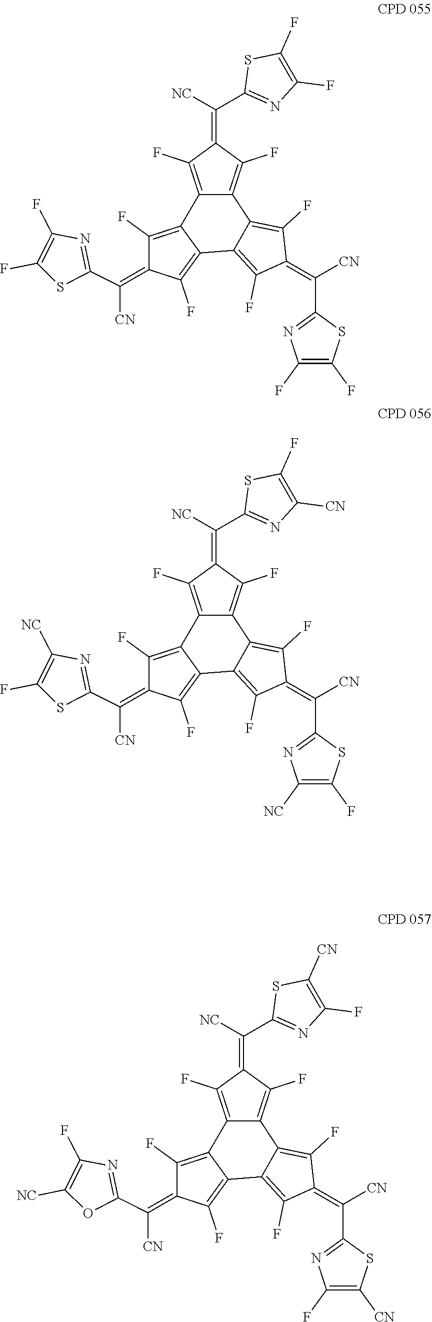 [SMILES: N#CC(=C1C(F)=c2c3c(c4c(c2=C1F)=C(F)C(=C(C#N)c1nc(F)c(F)s1)C=4F)=C(F)C(=C(C#N)c1nc(F)c(F)s1)C=3F)c1nc(F)c(F)s1 ; N#CC(=C1C(F)=c2c3c(c4c(c2=C1F)=C(F)C(=C(C#N)c1nc(C#N)c(F)s1)C=4F)=C(F)C(=C(C#N)c1nc(C#N)c(F)s1)C=3F)c1nc(C#N)c(F)s1 ; N#C/C(=C1/C(F)=c2c(c3c(c4c2=C(F)/C(=C(\C#N)c2nc(F)c(C#N)s2)C=4F)=C(F)/C(=C(\C#N)c2nc(F)c(C#N)s2)C=3F)=C1F)c1nc(F)c(C#N)o1]

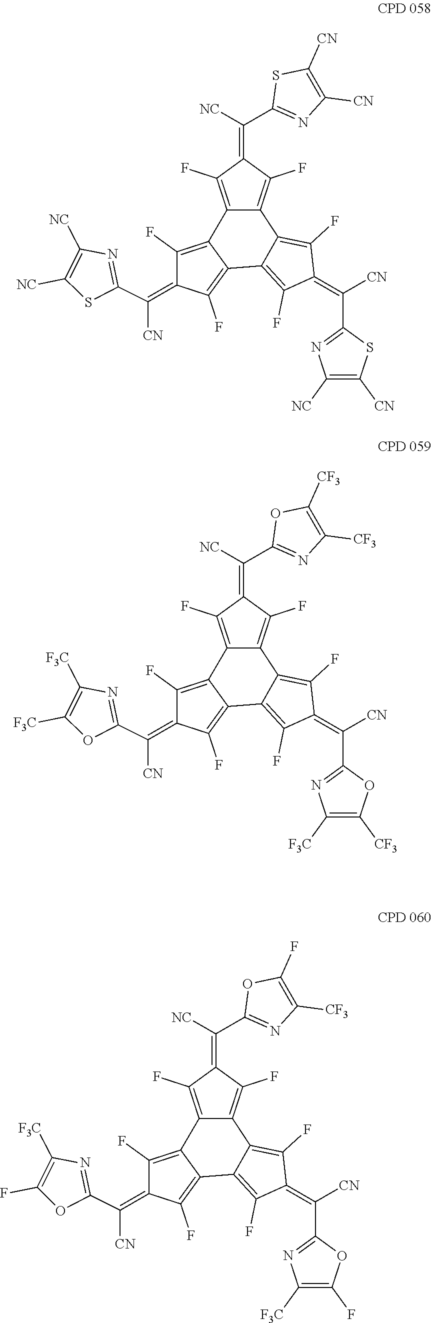 [SMILES: N#CC(=C1C(F)=c2c3c(c4c(c2=C1F)=C(F)C(=C(C#N)c1nc(C#N)c(C#N)s1)C=4F)=C(F)C(=C(C#N)c1nc(C#N)c(C#N)s1)C=3F)c1nc(C#N)c(C#N)s1 ; N#CC(=C1C(F)=c2c3c(c4c(c2=C1F)=C(F)C(=C(C#N)c1nc(C(F)(F)F)c(C(F)(F)F)o1)C=4F)=C(F)C(=C(C#N)c1nc(C(F)(F)F)c(C(F)(F)F)o1)C=3F)c1nc(C(F)(F)F)c(C(F)(F)F)o1 ; N#CC(=C1C(F)=c2c3c(c4c(c2=C1F)=C(F)C(=C(C#N)c1nc(C(F)(F)F)c(F)o1)C=4F)=C(F)C(=C(C#N)c1nc(C(F)(F)F)c(F)o1)C=3F)c1nc(C(F)(F)F)c(F)o1]

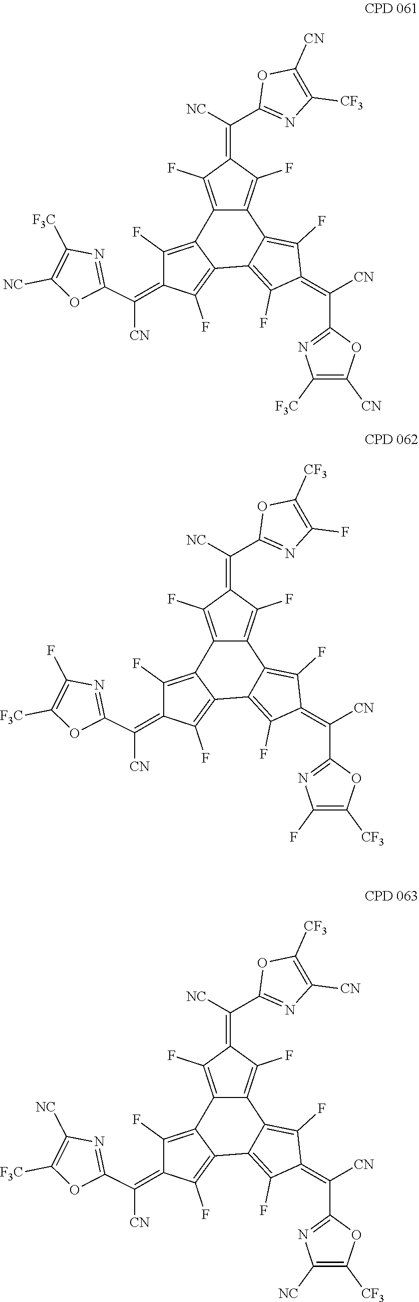 [SMILES: N#CC(=C1C(F)=c2c3c(c4c(c2=C1F)=C(F)C(=C(C#N)c1nc(C(F)(F)F)c(C#N)o1)C=4F)=C(F)C(=C(C#N)c1nc(C(F)(F)F)c(C#N)o1)C=3F)c1nc(C(F)(F)F)c(C#N)o1 ; N#CC(=C1C(F)=c2c3c(c4c(c2=C1F)=C(F)C(=C(C#N)c1nc(F)c(C(F)(F)F)o1)C=4F)=C(F)C(=C(C#N)c1nc(F)c(C(F)(F)F)o1)C=3F)c1nc(F)c(C(F)(F)F)o1 ; N#CC(=C1C(F)=c2c3c(c4c(c2=C1F)=C(F)C(=C(C#N)c1nc(C#N)c(C(F)(F)F)o1)C=4F)=C(F)C(=C(C#N)c1nc(C#N)c(C(F)(F)F)o1)C=3F)c1nc(C#N)c(C(F)(F)F)o1]

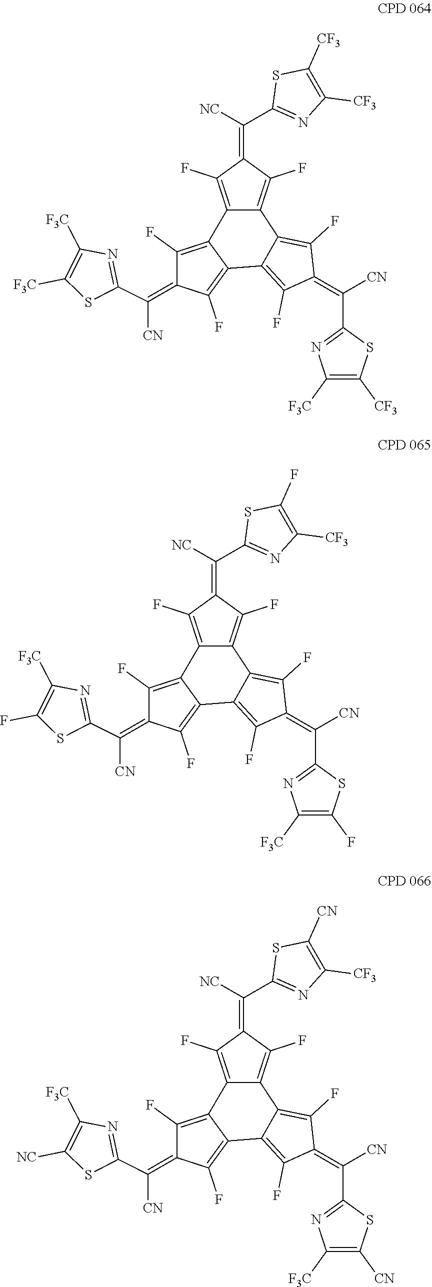 [SMILES: N#CC(=C1C(F)=c2c3c(c4c(c2=C1F)=C(F)C(=C(C#N)c1nc(C(F)(F)F)c(C(F)(F)F)s1)C=4F)=C(F)C(=C(C#N)c1nc(C(F)(F)F)c(C(F)(F)F)s1)C=3F)c1nc(C(F)(F)F)c(C(F)(F)F)s1 ; N#CC(=C1C(F)=c2c3c(c4c(c2=C1F)=C(F)C(=C(C#N)c1nc(C(F)(F)F)c(F)s1)C=4F)=C(F)C(=C(C#N)c1nc(C(F)(F)F)c(F)s1)C=3F)c1nc(C(F)(F)F)c(F)s1 ; N#CC(=C1C(F)=c2c3c(c4c(c2=C1F)=C(F)C(=C(C#N)c1nc(C(F)(F)F)c(C#N)s1)C=4F)=C(F)C(=C(C#N)c1nc(C(F)(F)F)c(C#N)s1)C=3F)c1nc(C(F)(F)F)c(C#N)s1]

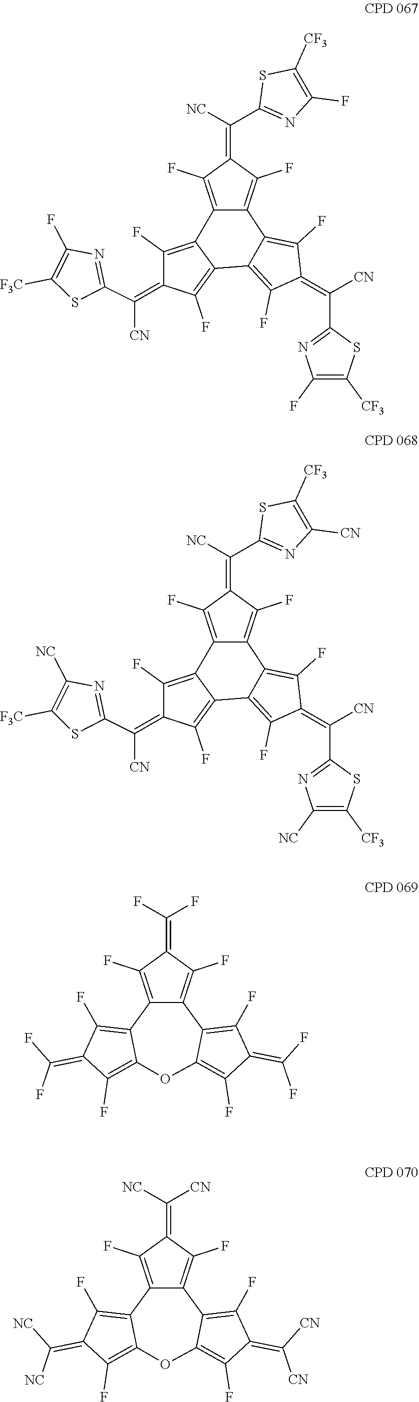 [SMILES: N#CC(=C1C(F)=c2c3c(c4c(c2=C1F)=C(F)C(=C(C#N)c1nc(F)c(C(F)(F)F)s1)C=4F)=C(F)C(=C(C#N)c1nc(F)c(C(F)(F)F)s1)C=3F)c1nc(F)c(C(F)(F)F)s1 ; N#CC(=C1C(F)=c2c3c(c4c(c2=C1F)=C(F)C(=C(C#N)c1nc(C#N)c(C(F)(F)F)s1)C=4F)=C(F)C(=C(C#N)c1nc(C#N)c(C(F)(F)F)s1)C=3F)c1nc(C#N)c(C(F)(F)F)s1 ; FC(F)=c1c(F)c2oc3c(F)c(=C(F)F)c(F)c3c3c(F)c(=C(F)F)c(F)c3c2c1F ; N#CC(C#N)=c1c(F)c2oc3c(F)c(=C(C#N)C#N)c(F)c3c3c(F)c(=C(C#N)C#N)c(F)c3c2c1F]

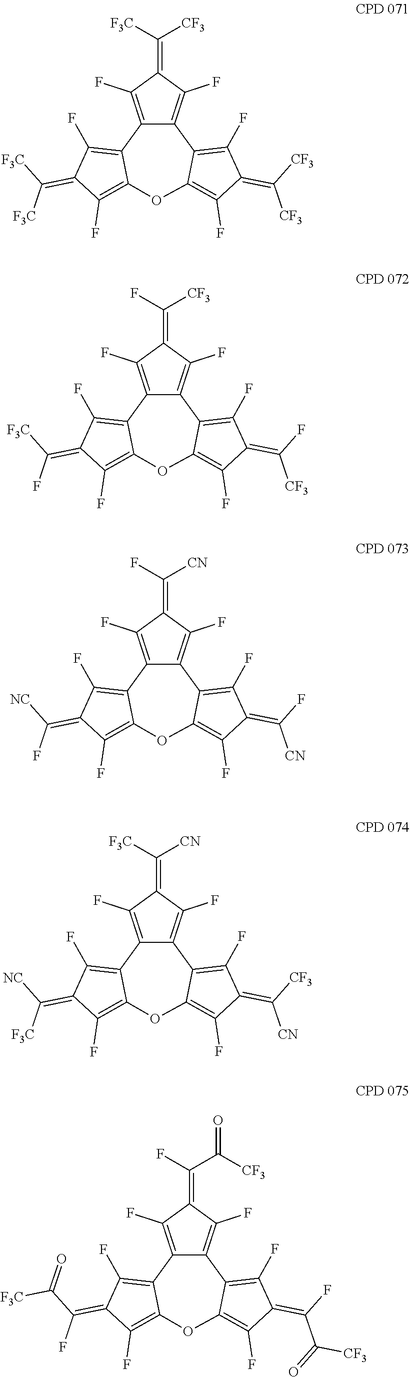 [SMILES: Fc1c(=C(C(F)(F)F)C(F)(F)F)c(F)c2c1oc1c(F)c(=C(C(F)(F)F)C(F)(F)F)c(F)c1c1c(F)c(=C(C(F)(F)F)C(F)(F)F)c(F)c21 ; F/C(=c1\c(F)c2oc3c(F)/c(=C(/F)C(F)(F)F)c(F)c3c3c(F)/c(=C(/F)C(F)(F)F)c(F)c3c2c1F)C(F)(F)F ; N#C/C(F)=c1/c(F)c2oc3c(F)/c(=C(/F)C#N)c(F)c3c3c(F)/c(=C(/F)C#N)c(F)c3c2c1F ; N#C/C(=c1/c(F)c2oc3c(F)/c(=C(\C#N)C(F)(F)F)c(F)c3c3c(F)/c(=C(\C#N)C(F)(F)F)c(F)c3c2c1F)C(F)(F)F ; O=C(/C(F)=c1/c(F)c2oc3c(F)/c(=C(/F)C(=O)C(F)(F)F)c(F)c3c3c(F)/c(=C(/F)C(=O)C(F)(F)F)c(F)c3c2c1F)C(F)(F)F]

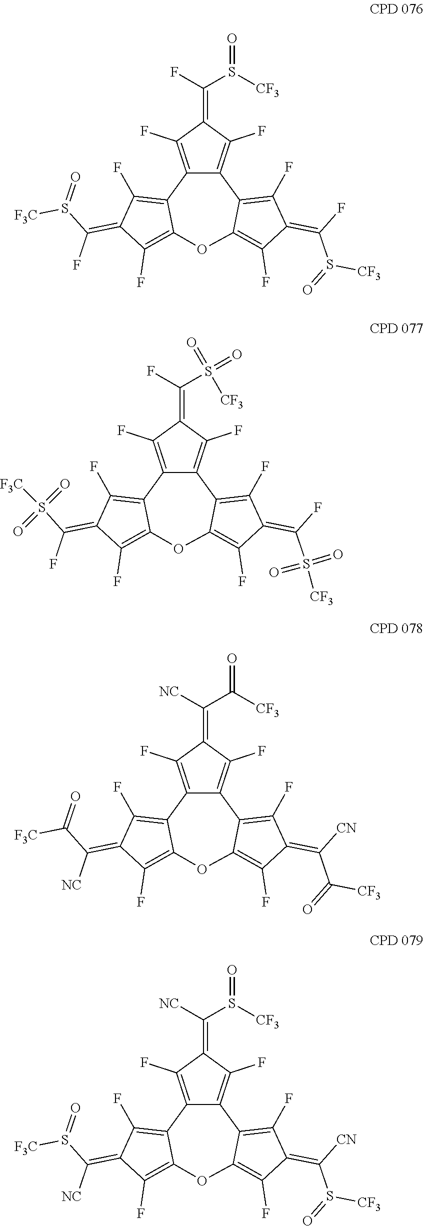 [SMILES: O=S(/C(F)=c1\c(F)c2oc3c(F)/c(=C(\F)S(=O)C(F)(F)F)c(F)c3c3c(F)/c(=C(\F)S(=O)C(F)(F)F)c(F)c3c2c1F)C(F)(F)F ; O=S(=O)(/C(F)=c1\c(F)c2oc3c(F)/c(=C(\F)S(=O)(=O)C(F)(F)F)c(F)c3c3c(F)/c(=C(\F)S(=O)(=O)C(F)(F)F)c(F)c3c2c1F)C(F)(F)F ; N#C/C(C(=O)C(F)(F)F)=c1/c(F)c2oc3c(F)/c(=C(\C#N)C(=O)C(F)(F)F)c(F)c3c3c(F)/c(=C(\C#N)C(=O)C(F)(F)F)c(F)c3c2c1F ; N#C/C(=c1/c(F)c2oc3c(F)/c(=C(\C#N)S(=O)C(F)(F)F)c(F)c3c3c(F)/c(=C(\C#N)S(=O)C(F)(F)F)c(F)c3c2c1F)S(=O)C(F)(F)F]

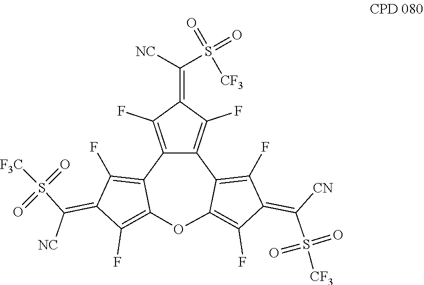 [SMILES: N#C/C(=c1/c(F)c2oc3c(F)/c(=C(\C#N)S(=O)(=O)C(F)(F)F)c(F)c3c3c(F)/c(=C(\C#N)S(=O)(=O)C(F)(F)F)c(F)c3c2c1F)S(=O)(=O)C(F)(F)F]

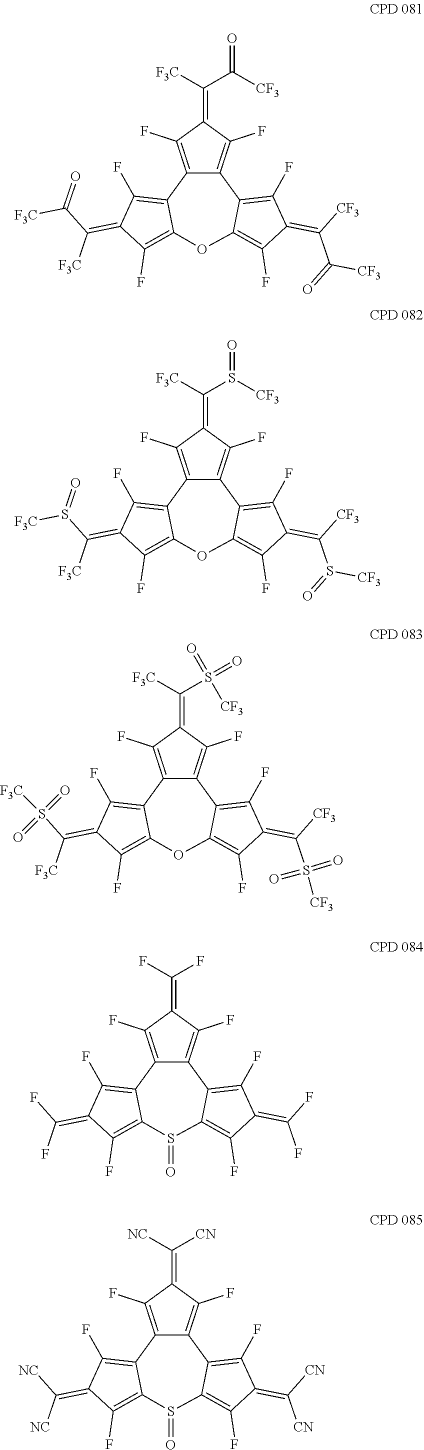

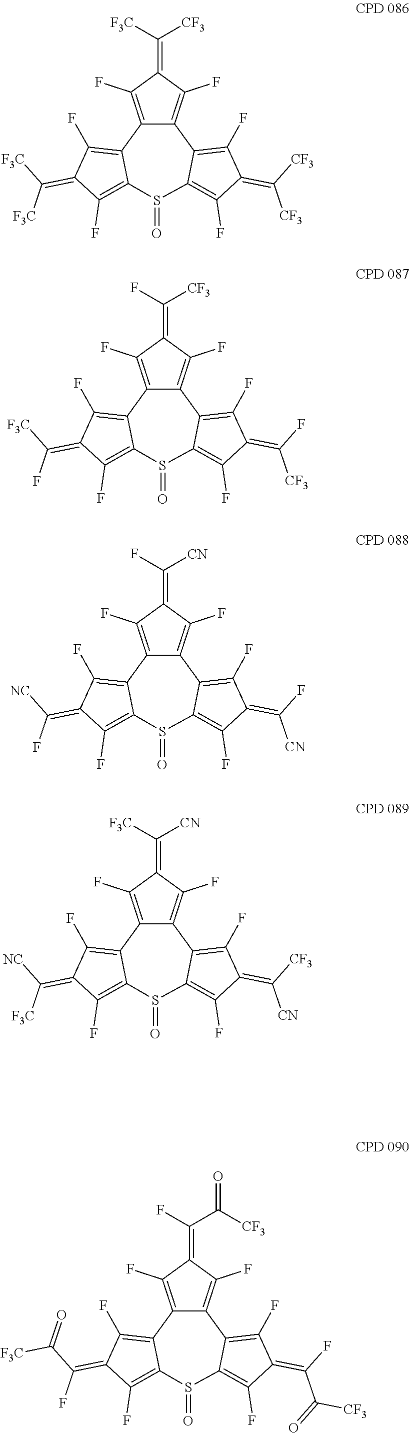

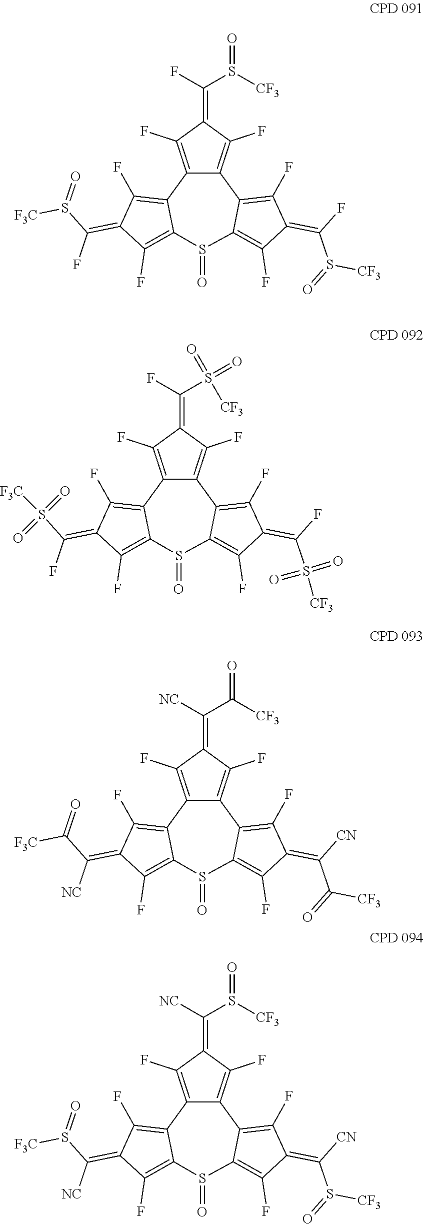 [SMILES: O=S1C2=C(F)/C(=C(/F)S(=O)C(F)(F)F)C(F)=C2C2=C(F)/C(=C(/F)S(=O)C(F)(F)F)C(F)=C2C2=C(F)/C(=C(/F)S(=O)C(F)(F)F)C(F)=C21 ; O=S1C2=C(F)/C(=C(/F)S(=O)(=O)C(F)(F)F)C(F)=C2C2=C(F)/C(=C(/F)S(=O)(=O)C(F)(F)F)C(F)=C2C2=C(F)/C(=C(/F)S(=O)(=O)C(F)(F)F)C(F)=C21 ; N#C/C(C(=O)C(F)(F)F)=C1/C(F)=C2C(=C1F)C1=C(F)/C(=C(/C#N)C(=O)C(F)(F)F)C(F)=C1S(=O)C1=C(F)/C(=C(/C#N)C(=O)C(F)(F)F)C(F)=C21 ; N#C/C(=C1/C(F)=C2C(=C1F)C1=C(F)/C(=C(/C#N)S(=O)C(F)(F)F)C(F)=C1S(=O)C1=C(F)/C(=C(/C#N)S(=O)C(F)(F)F)C(F)=C21)S(=O)C(F)(F)F]

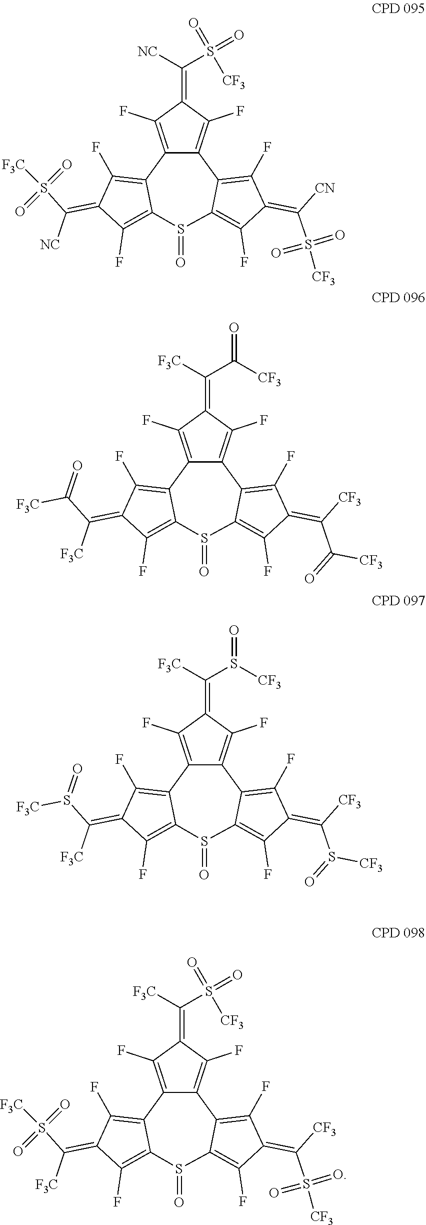

Furthermore one of the objectives of the present disclosure is to provide an electroluminescent device comprising a cathode, an anode, and an organic layer arranged between the cathode and the anode, wherein the organic layer comprises the above-mentioned organic compound.

The organic layer comprises a hole injection laxer, and the hole injection layer comprises the above-mentioned organic compound, wherein the hole injection layer further comprises a hole transport material containing triarylamine or carbazole as a matrix material in addition to the above-mentioned organic compound. An absolute value of a highest occupied orbital energy level (HOMO) of the hole transport material containing triarylamine or carbazole is 4.8-6.8 eV.

The compound of the present disclosure has a relatively low LUMO energy level, and the prepared red-light device has a low driving voltage, a better device luminous efficiency, and an improved lifetime. The above results indicate that the compound of the present disclosure can be used in an organic electroluminescent device as a hole injection layer material and has the potential to be applied to the OLED industry.

### DETAILED DESCRIPTION

The organic compound of the present disclosure has a structure shown in formula (1),

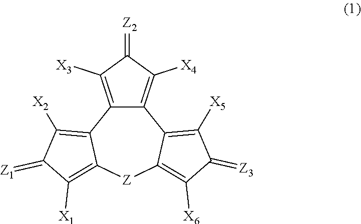

wherein
Z is a single bond, O, S, SO, or SO₂;
X₁-X₆ are each independently X, X-substituted C6-C30 aryl or X-substituted C2-C30 heteroaryl, and X is F, CN, or completely or partially fluorine-substituted C1-C4 fluoroalkyl;
Z₁-Z₃ are each independently O, S, Se, NR₁, or CR₂R₃;
wherein R₁-R₃ are each independently selected from the group consisting of hydrogen, deuterium, halogen, CN, substituted or unsubstituted C1-C20 alkyl, substituted or unsubstituted C3-C20 cycloalkyl, substituted or unsubstituted C1-C20 heteroalkyl, substituted or unsubstituted C7-C30 aralkyl, substituted or unsubstituted C1-C20 alkoxy, substituted or unsubstituted C6-C30 aryloxy, substituted or unsubstituted C2-C20 alkenyl, substituted or unsubstituted C2-C20 alkynyl, substituted or unsubstituted C6-C30 aryl, substituted or unsubstituted C3-C30 heteroaryl, substituted or unsubstituted C3-C20 alkylsilyl, substituted or unsubstituted C6-C20 arylsilyl, substituted or unsubstituted C1-C6 alkylsulfinyl, substituted or unsubstituted C1-C6 alkylsulfonyl, and substituted or unsubstituted C1-C6 alkylcarbonyl;
wherein at least one of R₁-R₃ is a group having an electron-withdrawing group;
wherein the substituents on R₁-R₃ are F, CN, completely or partially fluorine-substituted C1-C4 fluoroalkyl, completely or partially fluorine-substituted C1-C4 alkylsulfinyl, completely or partially fluorine-substituted C1-C4 alkylsulfonyl, or completely or partially fluorine-substituted C1-C4 alkylcarbonyl; and
wherein the heteroalkyl and heteroaryl contain at least one O, N or S heteroatom.

In an embodiment, Z is a single bond, and the organic compound has a structure shown in formula (2)

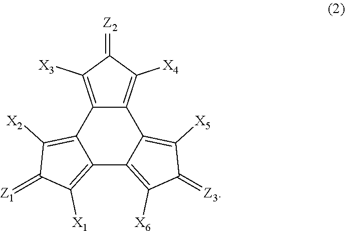

Hereinafter, examples of each of the groups of the compound as shown in formula (1) to formula (2) will be described.

It should be noted that in the present description, the “Ca-Cb” in the expression “substituted or unsubstituted Ca-Cb X group” represents the carbon number where the X group is unsubstituted, excluding the carbon number of substituents where the X group is substituted.

In an embodiment, C1-C10 alkyl is a linear or branched alkyl group, by way of example, methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, n-decyl and isomers thereof, etc., preferably methyl, ethyl, propyl, isopropyl, n-butyl, isobutyl, sec-butyl, and tert-butyl, and more preferably propyl, isopropyl, isobutyl, sec-butyl and tert-butyl.

In an embodiment, C3-C20 cycloalkyl include, by way of example, cyclopropyl, cyclobutyl, cyclopentyl, cyclohexyl, 1-adamantyl, 2-adamantyl, 1-norbornyl, 2-norbornyl etc., preferably cyclopentyl and cyclohexyl.

In an embodiment, C2-C10 alkenyl include, by way of example, vinyl, propenyl, allyl, 1-butadienyl, 2-butadienyl, 1-hexatrienyl, 2-hexatrienyl, 3-hexatrienyl, etc., preferably propenyl and allyl.

In an embodiment, C1-C10 heteroalkyl is linear or branched alkyl, cycloalkyl, etc., which contains atoms other than carbon and hydrogen, and include, by way of example, mercaptomethylimethanyl, methoxymethanol, ethoxymethanyl, tert-butoxymethanol, N,N-dimethylinethanyl, epoxybutanyl, epoxypentanyl, epoxyhexanyl, etc., preferably methoxymethanyl and epoxypentanyl.

In an embodiment, specific examples of aryl are phenyl, naphthalenyl, anthracenyl, phenanthrenyl, tetraphenyl, pyrenyl, chrysenyl, benzo[c]phenanthrenyl, benzo[g]chrysenyl, fluorenyl, benzofluorenyl, dibenzofluorenyl, biphenyl, terphenyl, quaterphenyl, fluoranthenyl, etc., preferably phenyl and naphthalenyl.

In an embodiment, specific examples of heteroaryl include, by way of example, pyrrolyl, pyrazinyl, pyridyl, pyrimidinyl, triazinyl, indolyl, isoindolyl, imidazolyl, furyl, benzofuranyl, isobenzofuranyl, dibenzofuranyl, dibenzothienyl, azadibenzofuranyl, azadibenzothienyl, diazadibenzofuranyl, diazadibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furazanyl, thienyl, benzothienyl, dihydroacridinyl, azacarbazolyl, diazacarbazolyl, quinazolinyl, etc., preferably pyridyl, pyrimidinyl, triazinyl, dibenzofuranyl, dibenzothienyl, azadibenzofuranyl, azadibenzothienyl, diazadibenzofuranyl, diazadibenzothienyl, carbazolyl, azacarbazolyl, and diazacarbazolyl.

In the description, the electron-withdrawing substituent refers to a group in which the electron cloud density on a benzene ring decreases after a substituent replaces a hydrogen on the benzene ring. Generally, the Hammett constant of such a substituent is a positive value, Generally, specific examples of electron-withdrawing substituents include, by way of example, nitro, cyano, sulfonic group, F, Cl, Br, I, trifluoromethyl, trifluoromethylsulfonyl, trifluoromethylsulfinyl, alkynyl, sulfonyl, sulfinyl, phosphonyl, aldehyde group, keto, ester group, carbonyl, pyrazinyl, pyridinyl, pyrimidinyl, triazinyl, quinolyl, isoquinolyl, quinoxalinyl, and alkyl, cycloalkyl and aromatic groups that contain the foregoing groups, etc. The Hammett constant of preferred substituents is preferably ≥0.05, particularly preferably ≥0.3, especially preferably ≥0.5. Examples are preferably CN, F, CF3, pyridinyl, pyrimidinyl, and triazinyl.

The following examples are only for the convenience of understanding the present disclosure and should not be regarded as specific limitations to the present disclosure.

Raw materials, solvents, etc., involved during the synthesis of compounds in the present disclosure are all purchased from suppliers familiar to those skilled in the art, such as Alfa and Acros.

### Synthesis of Compound CPD001

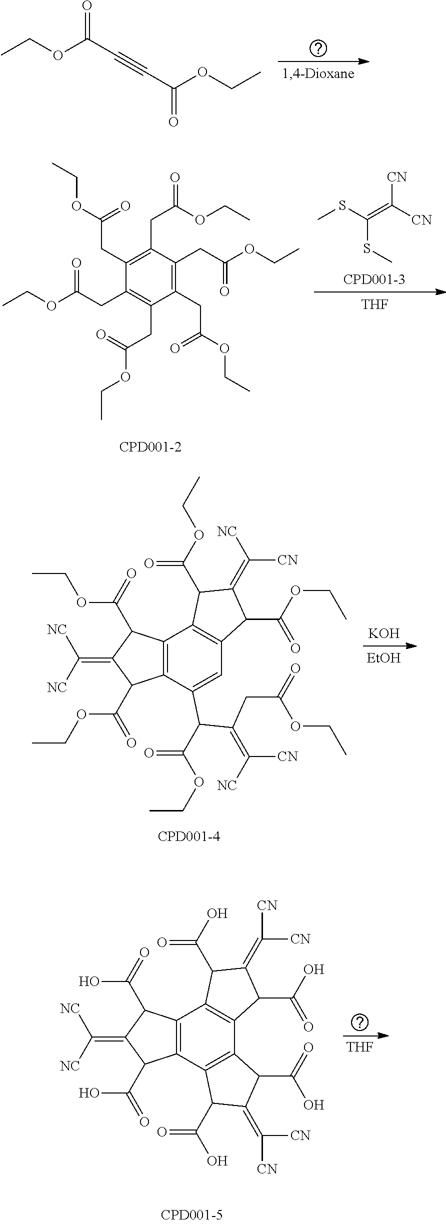

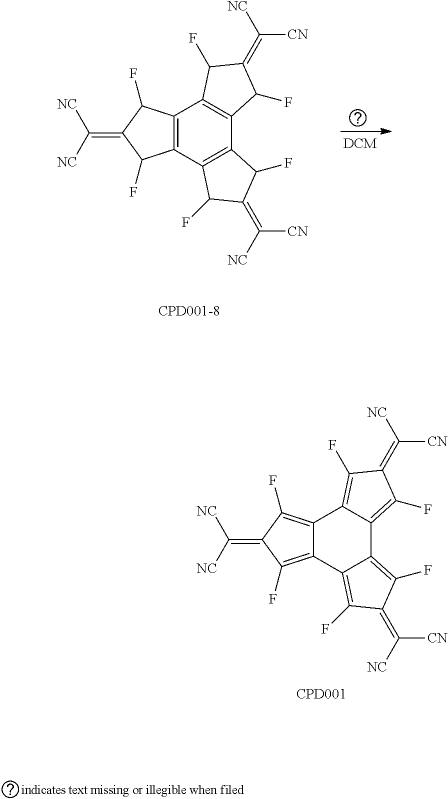

### Synthesis of Compound CPD001-2

CPD001-1 (50 g, 293.83 mmol), dicarbonylclopentadienyl cobalt (2.64 g, 14.69 mmol), and 1,4-dioxane (500 mL) were added to a 1000 mL three-necked round-bottom flask, and the flask was displaced with nitrogen three times. After heating to 110° C. and refluxed for 24 h, TLC monitored (ethyl acetate:petroleum ether=1:20) that the raw material CPD001-1 was completely consumed.

The reaction solution was concentrated to remove 1,4-dioxane, added with dichloromethane (500 mL), washed with deionized water (200 mL*3), and then successively subjected to liquid separation, concentration, purification by silica gel column chromatography (200-300 mesh silica gel, acetate:petroleum ether=1:50 as an eluent), elution and concentration, to obtain Compound CPD001-2 as a colorless liquid (35.52 g, purity: 99.63%, yield: 61.00%). Mass spectrum: 595.4 (M+H).

¹H NMR (400 MHz, CDCl₃) δ 4.13 (q, J=11.91 Hz, 12H), 3.71 (s, 12H), 1.21 (L, J=11.8 Hz, 18H).

### Synthesis of Compound CPD001-4

CPD001-2 (35.00 g, 58.86 mmol) and dry tetrahydrofuran (350 mL) were added to a 1000 mL three-necked round-bottom flask and cooled to 0° C., and then added with sodium hydride with a mass fraction of 60% (18.86 g, 470.88 mmol) in portions and stirred at 0° C. for 30 minutes, followed by the addition of CPD001-3 (50.10 g, 294.29 mmol). The mixture was returned to room temperature and stirred overnight for 24 h. TLC monitored (ethyl acetate:petroleum ether=1:20) that the raw material CPD001-2 was completely consumed.

After cooling to 5° C., deionized water (100 mL) was added to quench the reaction. The reaction solution was concentrated to remove the solvent, and a large amount of solid was precipitated. After suction filtration, the solid was sprinkled with iced ethanol (100 mL) and recrystallized twice with 10-fold ethanol, and the filter cake was dried in vacuum at 50° C. for 4 h to obtain compound CPD001-4 as a light yellow solid (37.5 g, purity: 99.57%, yield: 78.05%). Mass spectrum: 817.22 (M+H).

¹H NMR (400 MHz, CDCl₃) δ 5.49 (s, 6H), 4.21 (q, J=11.8 Hz, 12H), 1.20 (t, J=11.8 Hz, 18H).

### Synthesis of Compound CPD001-5

CPD001-4 (15.00 g, 18.36 mmol), ethanol (150 mL), potassium hydroxide (10.30 g, 183.65 mmol), and deionized water (46 mL) were added to a 500 mi, three-necked round-bottom flask, and the reaction system was then heated to 80° C. and reacted for 10 h. TLC (methanol:dichloromethane=1:20 as a developing agent) monitored that the raw material CPD001-4 was completely consumed.

The reaction was cooled to 5° C., and the pH of the reaction system was adjusted to 6 by adding 10% hydrochloric acid, whereupon a large amount of a solid was precipitated. After suction filtration, the filter cake was pulped at room temperature with a mixed solvent of deionized water (200 mL) and methanol (200 mL) for 1 h. After suction filtration, the filter cake was sprinkled with deionized water (100 mL) and with iced methanol (80 mL) and dried in vacuum at 50° C. for 24 hours to obtain CPD001-5 as a yellow solid (10.86 g, purity: 99.87%, yield: 91.21%). Mass spectrum: 649.25 (M+H).

### Synthesis of Compound CPD001-6

CPD001-5 (10.00 g, 15.42 mmol), sodium bicarbonate (12.95 g, 154.20 mmol), selectfluor (43.70 g, 123.36 mmol), and tetrahydrofuran (100 mL) were added to a 500 mL three-necked round-bottom flask and stirred at room temperature overnight for 28 h. TLC (methanol dichloromethane=1:20 as a developing agent) monitored that the raw material CPD001-5 was completely consumed.

The reaction was cooled to 5° C., methanol was added, and the mixture was retuned to room temperature and pulped for 30 minutes. After suction filtration, the filter cake was washed with iced methanol (40 mL) and dried in vacuum at 50° C. to obtain CPD001-6 as a yellow solid (4.96 g, purity: 99.90%, yield: 65.37%). Mass spectrum: 493.46 (M+H).

### Synthesis of Compound CPD001

CPD001-6 (4.90 g, 9.95 mmol), dichloromethane (75 mL), potassium hydroxide (6.70 g, 119.40 mmol), deionized water (30 mL), and potassium ferricyanide (26.21 g, 79.60 mmol) were added to a 500 mL three-necked round-bottom flask and stirred at room temperature for 24 h.

After suction filtration, the filter cake was sprinkled with dichloromethane (80 mL), recrystallized twice with 5-fold chloroform, and dried in vacuum at 50° C. for 8 hours to obtain target Compound CPD001 as a yellowish brown solid (3.22 g, purity: 99.90%, yield: 66.54%). 3.22 g of crude CPD001 was sublimated and purified to obtain sublimated pure CPD001 (1.03 g, purity: 99.90%, yield: 31.98%). Mass spectrum: 487.02 (M+H).

¹³C NMR (100 MHz, CDCl₃) δ 134.26, 124.88, 113.35, 82.58.

¹⁹F NMR (377 MHz, CDCl₃) δ −146.20.

### Synthesis of Compound CPD003

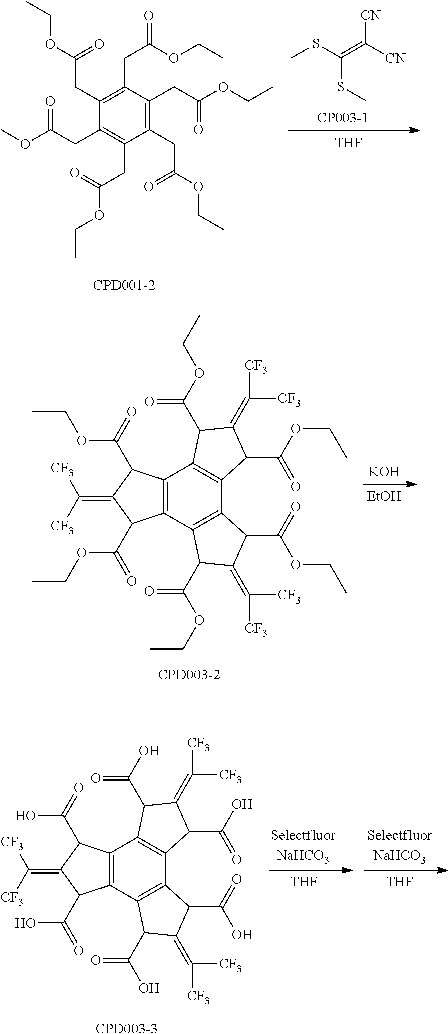

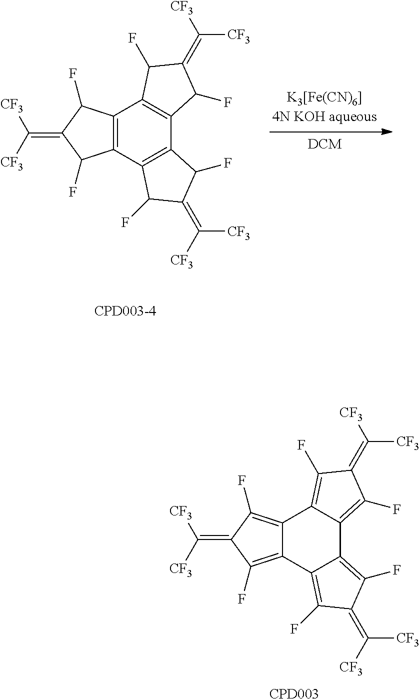

### Synthesis of Compound CPD003-2

Referring to the synthesis and purification method for Compound CPD001-4, only by changing the corresponding raw materials, Compound CPD003-2 as a yellow solid (28.16 g, purity: 99.63%, yield: 75.35%) was obtained. Mass spectrum: 1075.20 (M+H).

### Synthesis of Compound CPD003-3

Referring to the synthesis and purification method for Compound CPD001-5, only by changing the corresponding raw materials, Compound CPD003-3 as a yellow solid (17.47 g, purity: 99.71%, yield: 89.62%) was obtained Mass spectrum: 907.22 (M+H).

### Synthesis of Compound CPD003-4

Referring to the synthesis and purification method for Compound CPD001-6, only by changing the corresponding raw, materials, Compound CPD903-4 as a yellow solid (8.19 g, purity: 99.88%, yield: 55.36%) was obtained. Mass spectrum 751.42 (M+H).

### Synthesis of Compound CPD903

Referring to the synthesis and purification method for Compound CPD001 only by changing the corresponding raw materials, target Compound CPD003 as a yellowish brown solid (5.18 g, purity: 99.91%, yield: 62.17%) was obtained. 5.18 g of crude CPD003 was sublimated and purified to obtain sublimated pure CPD003 (2.03 g, purity: 99.91%, yield: 39.19%). Mass spectrum 745.02 (M+H).

¹³C NMR (100 MHz, CDCl₃) δ 170.22, 124.97, 119.76, 111.43.

¹⁹F NMR (377 MHz, CDCl₃) δ −61.80, −146.20.

### Synthesis of Compound CPD016

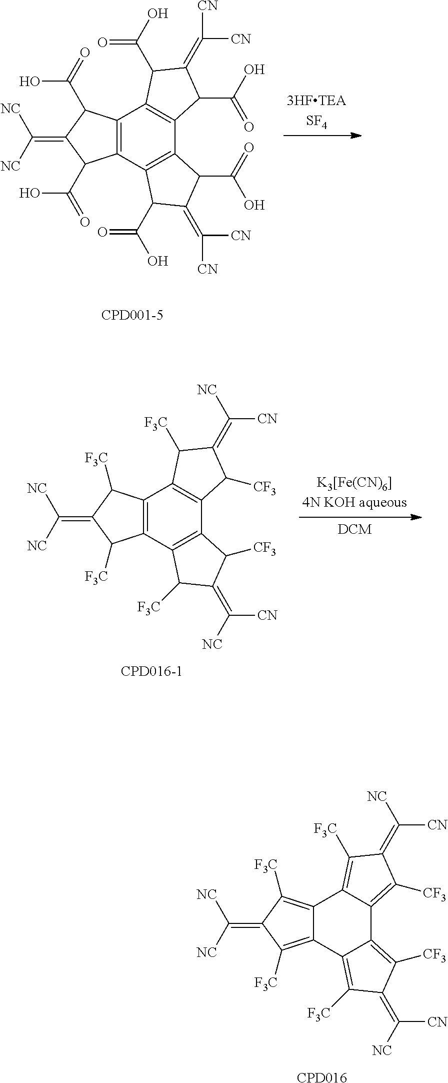

### Synthesis of Compound CPD016-1

CPD001-5 (16.20 g, 24.98 mmol) and triethylamine hydrofluoride (80.54 g, 499.60 mmol) were added to a stainless steel autoclave, into which sulfur tetrafluoride gas (53.98 g, 499.60 mmol) was then introduced, and the resulting mixture was then heated to 80° C. and reacted for 24 h.

The reaction system was cooled to room temperature, the fume hood was turned on for forced ventilation, the autoclave was slowly depressurized and evacuated, and the reaction solution was slowly dropwise added to deionized water at 5° C. (500 mL) and naturally returned to room temperature. After stirring for 1.5 hours, a large amount of dispersed solid was precipitated, and after suction filtration, the solid was washed with deionized water (200 mL) and with iced methanol (50 mL) and dried in vacuum at 50° C. to obtain CPD016-1 as a yellow solid (10.76 g, purity: 99.89%, yield: 54.34%). Mass spectrum: 793.04 (M+H).

### Synthesis of Compound CPD016

Referring to the synthesis and purification method for Compound CPD001, only by changing the corresponding raw materials, target Compound CPD016 as a yellowish brown solid (4.03 g, purity: 99.91%, yield: 60.51%) was obtained. 4.03 g of crude CPD016 was sublimated and purified to obtain sublimated pure CPD016 (1.38 g, purity: 99.91%, yield: 34.24%), Mass spectrum: 787.04 (M+H).

¹³C NMR (100 MHz, CDCl₃) δ 172.08, 121.05, 120.10, 118.42, 113.35, 107.82.

¹⁹F NMR (377 MHz, CDCl₃) δ −59.80.

### Synthesis of Compound CPD031

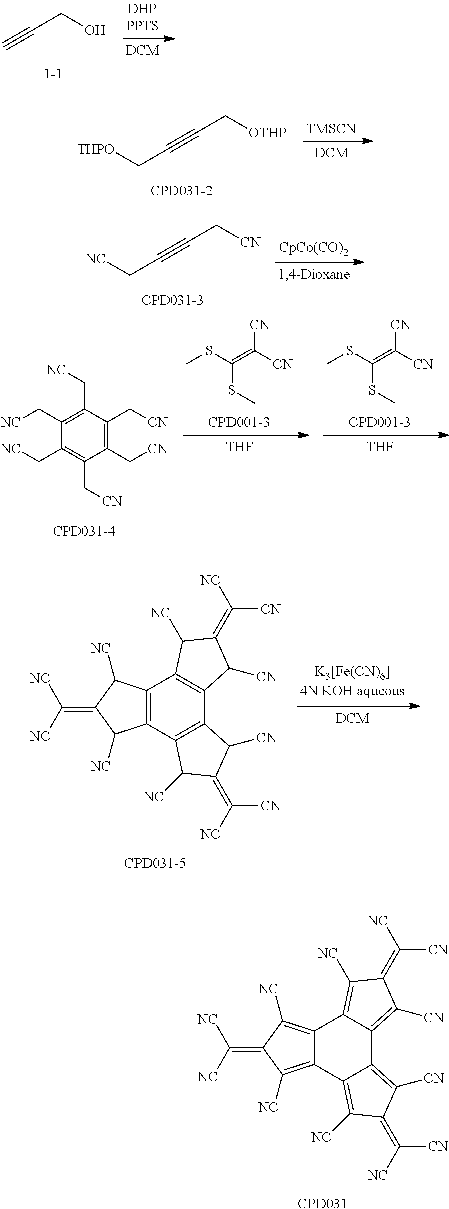

### Synthesis of Compound CPD031-2

CPD031-1 (50.00 g, 0.58 mol), 3,4-dihydro-2H-pyran (14637 g, 1.74 mol), pyridinium p-toluenesulfonate (4.37 g, 17.40 mmol), and dichloromethane (500 mL were added to a 1000 mL three-necked round-bottom flask. TLC monitored (ethyl acetate:petroleum ether=1:20) that after 2 hours, the raw material CPD031-1 was completely consumed.

The reaction solution was added with dichloromethane (200 mL), washed with deionized water (300 mL*3), and then successively subjected to liquid separation, concentration, purification by silica gel column chromatography (200-300 mesh silica gel, acetate:petroleum ether=1:40 as an eluent). After concentration, Compound CPD031-2 was obtained as a light yellow liquid (132.94 g, purity: 99.83%, yield: 90.00%). Mass spectrum: 255.16 (M+H).

### Synthesis of Compound CPD031-3

To a 2000 mL three-necked round-bottom flask, CPD031-2 (120.00 g, 0.47 mol) and dry dichloromethane (1200 mL) were added, and the flask was displaced with nitrogen three times. The reaction system was then cooled to 0° C. while stirring, trimethylcyanosilane (140.43 g, 1.42 mol) was then added dropwise, and finally, stannic chloride (240 mL, 1.0 M solution in CH₂Cl₂, 0.24 mmol) was added dropwise. Subsequently, the reaction was returned to room temperature and reacted overnight. TLC monitored (ethyl acetate:petroleum ether=1:10) that the raw material CPD031-2 was completely consumed.

An aqueous solution of potassium carbonate was added to quench the reaction. The reaction solution was maintained at room temperature and stirred for 30 minutes, and then successively subjected to liquid separation, washing with deionized water (300 mL*2), liquid separation, silica gel column chromatography (200-300 mesh silica gel, ethyl acetate:petroleum ether=1:20 as an eluent), and concentration, to obtain CPD031-3 as a light yellow liquid (43.31 g, purity: 99.85%, yield: 88.16%). Mass spectrum: 105.04 (M+H).

### Synthesis of Compound CPD031-4

Referring to the synthesis and purification method for Compound CPD001-2, only by changing the corresponding raw materials, target Compound CPD031-4 was obtained (31.21 g, purity: 99.58%, yield: 57.72%). Mass spectrum: 313.12 (M+H).

### Synthesis of Compound CPD031-5

Referring to the synthesis and purification method for Compound CPD001-4, only by changing the corresponding raw materials, Compound CPD031-5 as a yellow solid (15.16 g, purity: 99.64%, yield: 73.33%) was obtained Mass spectrum: 535.08 (M+H).

### Synthesis of Compound CPD031

Referring to the synthesis and purification method for Compound CPD001, only by changing the corresponding raw materials, target Compound CPD031 as a yellowish brown solid (6.05 g, purity: 99.90%, yield: 55.19%) was obtained. 6.05 g of crude CPD031 was sublimated and purified to obtain sublimated pure CPD031 (2.34 g, purity: 99.91%, yield: 38.67%). Mass spectrum: 529.24 (M+H).

¹³C NMR (100 MHz, CDCl₃) δ 14935, 143.10, 113.35, 106.01, 99.44, 67.86.

### Synthesis of Compound CPD032

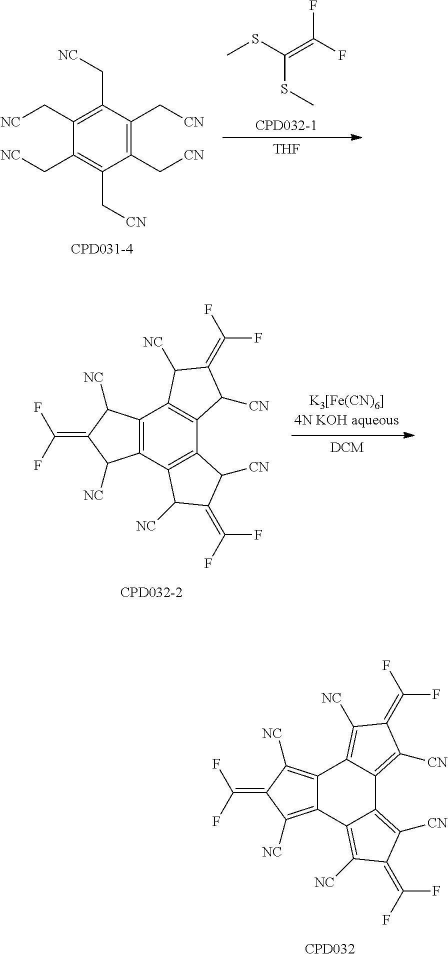

### Synthesis of Compound CPD032-1

Referring to the synthesis and purification method for Compound CPD001-4, only by changing the corresponding raw materials, target Compound CPD032-1 (8.29 g purity: 99.88%, yield: 71.41%) was obtained. Mass spectrum: 493.16 (M+1).

### Synthesis of Compound CPD032

Referring to the synthesis and purification method for Compound CPD001, only by changing the corresponding raw materials, target Compound CPD032 as a yellowish brown solid (5.83 g, purity: 99.92%, yield: 48.83%) was obtained. 5.83 g of crude CPD032 was sublimated and purified to obtain sublimated pure CPD032 (1.69 g, purity: 99.92% yield: 28.98%), Mass spectrum: 487.02 (M+H).

¹³C NMR (100 MHz, CDCl₃) δ 150.30, 138.39, 114.48, 107.85, 80.81.

¹⁹F NMR (377 MHz, CDCl₃) δ −65.70.

### Synthesis of Compound CPD046

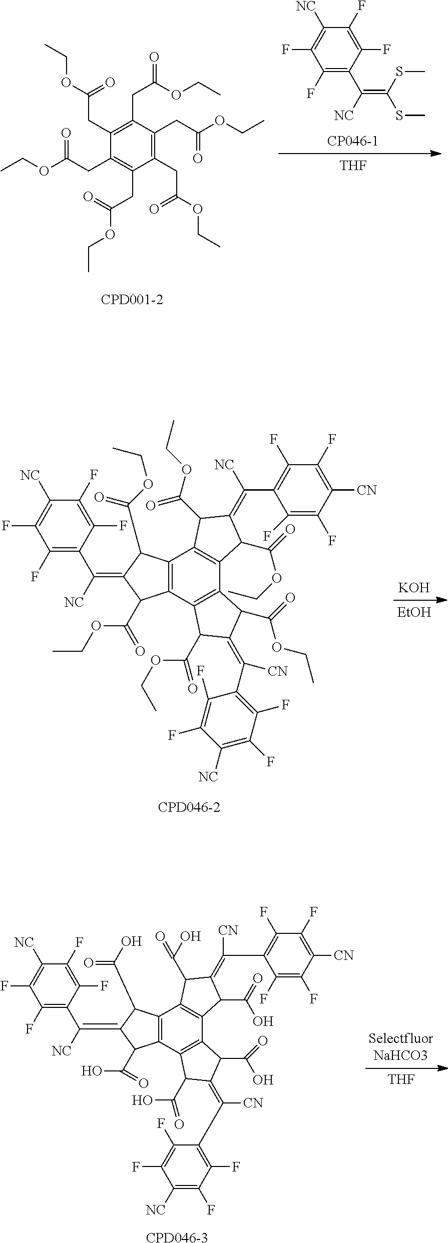

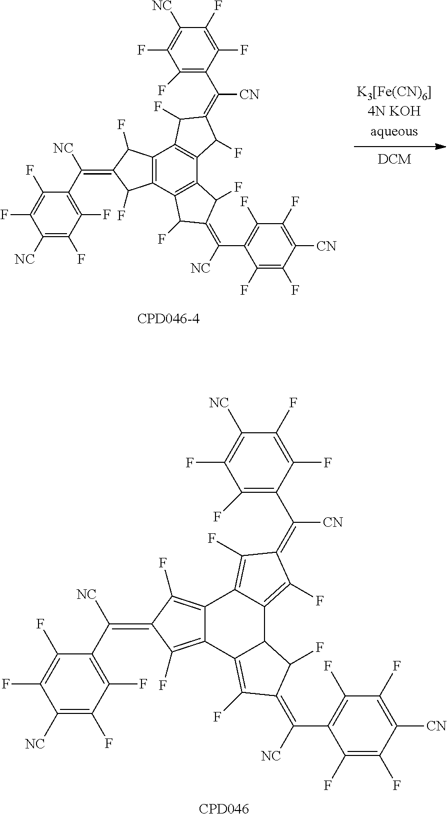

### Synthesis of Compound CPD046-2

Referring to the synthesis and purification method for Compound CPD001-4, only by changing the corresponding raw materials, Compound CPD046-2 as a yellow solid (25.33 g, purity: 99.50%, yield: 77.47%) was obtained. Mass spectrum: 1261.26 (M+H).

### Synthesis of Compound CPD046-3

Referring to the synthesis and purification method for Compound CPD001-5, only by changing the corresponding raw materials, Compound CPD046-3 as a yellow solid (13.44 g purity: 99.75%, yield: 91.25%) was obtained. Mass spectrum: 1093.23 (M+H).

### Synthesis of Compound CPD046-4

Referring to the synthesis and purification method for Compound CPD001-6, only by changing the corresponding raw materials, Compound CPD046-4 as a yellow solid (8.53 g, purity: 99.91%, yield: 58.13%) was obtained. Mass spectrum: 937.24 (M+H).

### Synthesis of Compound CPD046

Referring to the synthesis and purification method for Compound CPD001, only by changing the corresponding raw materials, target Compound CPD046 as a yellowish brown solid (6.12 g, purity: 99.91%, yield: 50.14%) was obtained. 6.12 g of crude CPD046 was sublimated and purified to obtain sublimated pure CPD046 (2.06 g, purity: 99.91%, yield: 33.66%). Mass spectrum: 931.42 (M+H).

¹³C NMR (100 MHz, CDCl₃) δ 145.60, 143.59, 142.00, 139.99, 131.02, 124.77, 110.99, 108.09, 107.01, 86.28.

¹⁹F NMR (377 MHz, CDCl₃) δ −138.50, −143.10, −146.20.

### Synthesis of Compound CPD058

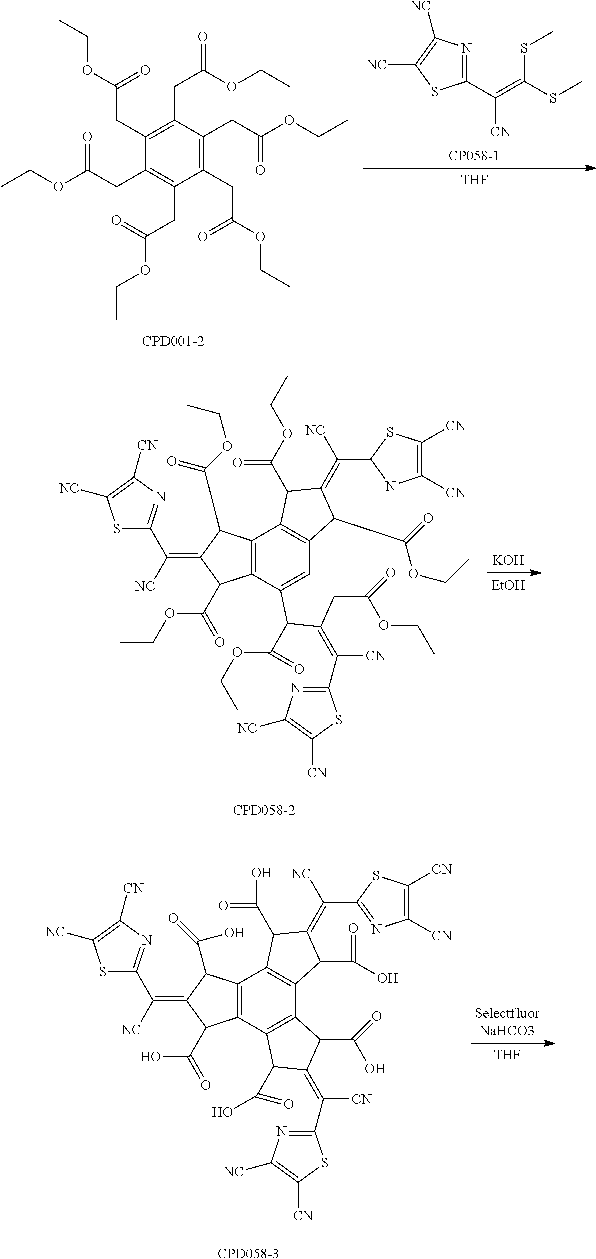

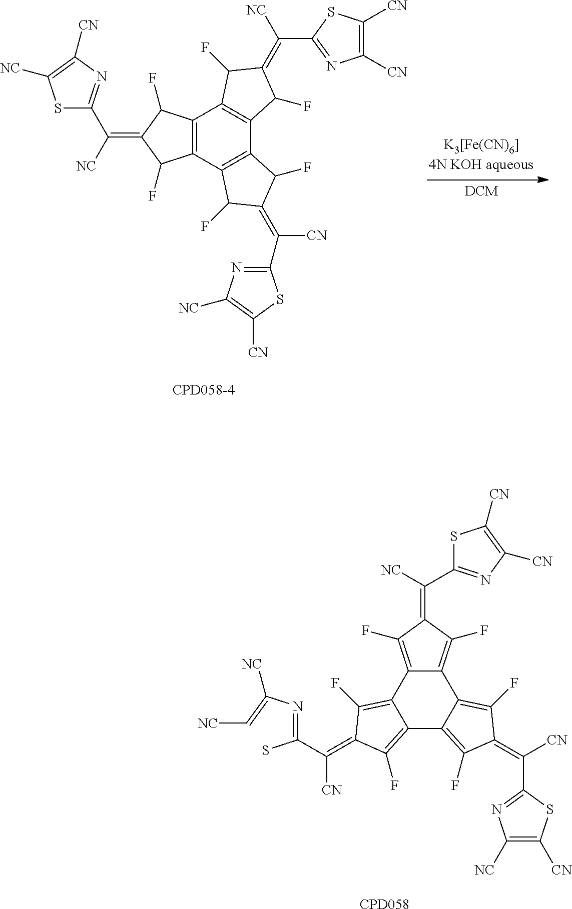

### Synthesis of Compound CPD058-2

Referring to the synthesis and purification method for Compound CPD001-4, only by changing the corresponding raw materials, compound CPD058-2 as a yellow solid (27.59 g, purity: 99.50%, yield: 80.01%) was obtained. Mass spectrum: 1141.27 (M+H).

### Synthesis of Compound CPD058-3

Referring to the synthesis and purification method for Compound CPD001-5, only by changing the corresponding raw materials, Compound CPD058-3 as a yellow solid (18.03 g, purity: 99.77%, yield: 93.21%) was obtained Mass spectrum: 973.25 (M+H).

### Synthesis of Compound CPD058-4

Referring to the synthesis and purification method for Compound CPD001-6, only by changing the corresponding raw materials, Compound CPD058-4 as a yellow solid (8.26 g, purity: 99.89%, yield: 62.11%) was obtained. Mass spectrum: 817.20 (M+H).

### Synthesis of Compound CPD058

Referring to the synthesis and purification method for Compound CPD001, only by changing the corresponding raw materials, target Compound CPD058 as a yellowish brown solid (4.83 g, purity: 99.91%, yield: 42.92%) was obtained. 4.83 g of crude CPD058 was sublimated and purified to obtain sublimated pure CPD058 (1.25 g, purity: 99.91%, yield: 25.87%). Mass spectrum: 811.62 (M+H).

¹³C NMR (100 MHz, CDCl₃) δ 156.50, 148.75, 139.36, 132.37, 125.98, 125.50, 124.39, 116.83, 114.98, 114.53.

¹⁹F NM R (377 MHz, CDCl₃) δ −146.20.

### Synthesis of Compound CPD070

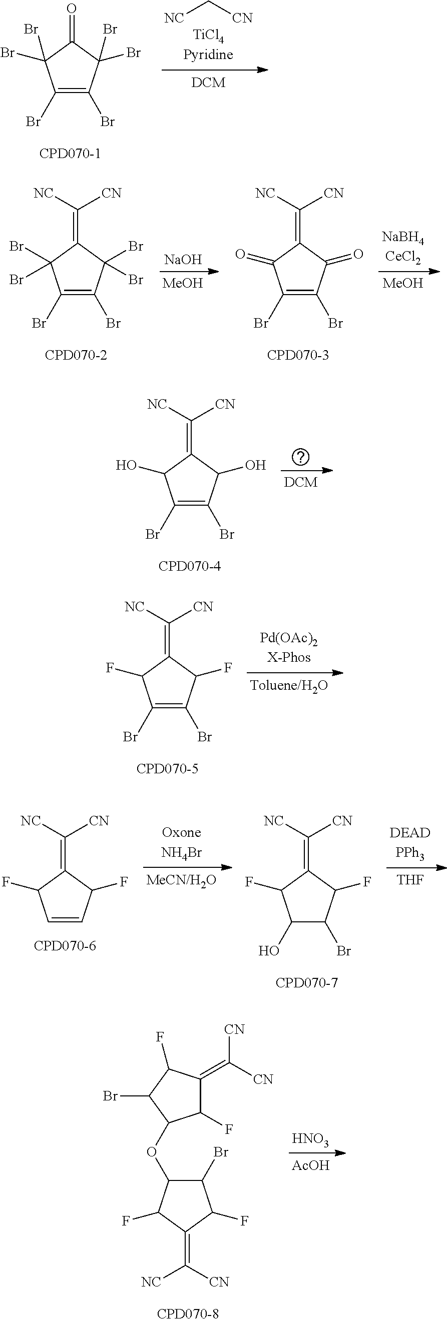

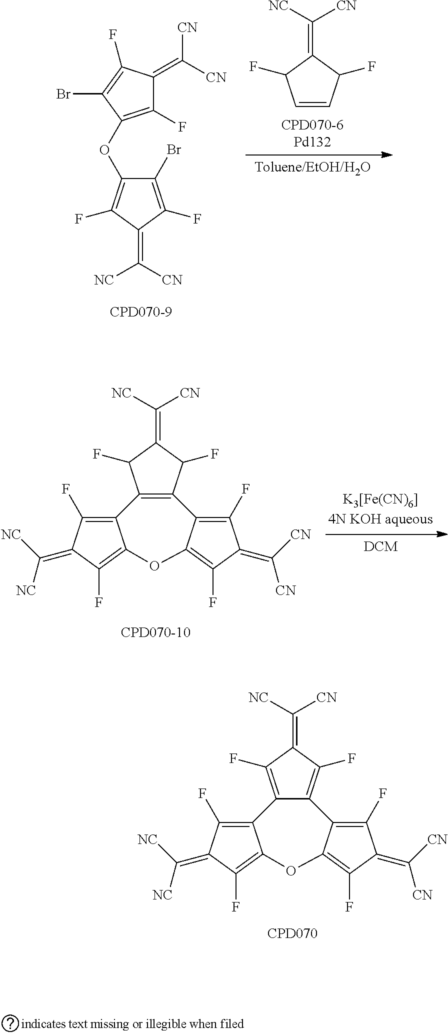

### Synthesis of Compound CPD070-2

CPD070-1 (50.00 g. 90.01 mmol), propanedinitrile (11.89 g, 180.03 mmol), and dichloromethane (500 mL) were added to a 1000 mL three-necked round-bottom flask, followed by the addition of pyridine (7.12 g, 90.01 mmol) and titanium tetrachloride (15.23 g, 90.01 mmol), and the flask was displaced with nitrogen three times. The resulting mixture was maintained at room temperature and stirred overnight. TLC monitored (ethyl acetate:petroleum ether=1:18) that the raw material CPD070-1 was completely consumed.

The reaction solution was washed by adding deionized water (300 mL*3), and successively subjected to liquid separation, concentration, purification by silica gel column chromatography (200-300 mesh silica gel, acetate:petroleum ether=1:30 as an eluent) and concentration, to obtain CPD070-2 as a light yellow liquid (30.42 g, purity: 99.53%, yield: 56.00%). Mass spectrum: 598.56 (M+H1).

### Synthesis of Compound CPD070-3

CPD070-2 (29.00 g, 48.05 mmol), sodium hydroxide (13.45 g, 336.36 mmol), methanol (290 mL), and deionized water (80 mL) were added to a 1000 mL three-necked round-bottom flask, and the flask was displaced with nitrogen three times. The resulting mixture was heated to 60° C. and stirred overnight. TLC monitored (ethyl acetate:petroleum ether=1:8) that the raw material CPD070-2 was completely consumed.

The reaction solution was concentrated to remove the solvent, added with dichloromethane (500 mL), washed with deionized water (150 mL*3), and successively subjected to liquid separation, concentration, purification by silica gel column chromatography (200-300 mesh silica gel, acetate:petroleum ether=1:10 as an eluent), and concentration, to obtain CPD070-3 as a light yellow liquid (12.17 g, purity: 99.64%, yield: 80.16%). Mass spectrum: 315.02 (M+H).

### Synthesis of Compound CPD070-4

CPD070-3 (12.00 g, 37.99 mmol), anhydrous cereous chloride (18.72 g, 75.97 mmol), and methanol (120 mL) were added to a 500 mL three-necked round-bottom flask. The reaction system was cooled to 5° C., sodium borohydride (2.43 g, 75.97 mmol) was added in portions, and the reaction system was maintained at 5° C. for 30 minutes. TLC monitored (ethyl acetate:petroleum ether=1:5) that the raw material CPD070-3 was completely consumed.

The reaction solution was concentrated to remove the solvent, added with dichloromethane (500 mL), washed with deionized water (150 mL*3), and successively subjected to liquid separation, and concentration, to obtain CPD070-4 as a light yellow liquid (11.58 g, purity: 99.63%, yield: 95.25%). Mass spectrum: 319.06 (M+H). The crude product was directly used in the next step.

### Synthesis of Compound CPD070-5

CPD070-4 (11.00 g 34.38 mmol) and dry dichloromethane (110 mL) were added to a 500 mL three-necked round-bottom flask. The reaction system was cooled to −10° C., bis(2-methoxyethyl)aminosulfur trifluoride (18.94 g, 85.59 mmol) was slowly dropwise added, and the mixture was maintained at −10° C. and reacted for 10 minutes. TLC monitored (ethyl acetate:petroleum ether=1:5) that the raw material CPD070-4 was completely consumed.

10 mL of methanol was dropwise added to quench the reaction. The reaction solution was washed with deionized water (50 mL*3), and successively subjected to liquid separation, concentration, purification by silica gel column chromatography (200-300 mesh silica gel, acetate:petroleum ether=1:10 as an eluent) and concentration, to obtain CPD070-5 as a light yellow liquid (8.73 g, purity: 99.48%, yield: 78.36%). Mass spectrum: 323.14 (M+H).

### Synthesis of compound CPD070-6

CPD070-5 (8.50 g, 26.24 mmol), potassium phosphate (16.71 g, 78.72 mmol), palladium acetate (176.74 mg, 0.79 mmol), 2-dicyclohexylphosphine-2′,4′,6′-triisopropyl biphenyl (0.75 g, 1.58 mmol), toluene (120 ml), and deionized water (40 ml) were added to a 500 mL three-necked round-bottom flask, and the flask was displaced with nitrogen three times. The reaction system was heated to 120° C. and refluxed overnight for 16 hours. TLC monitored (ethyl acetate:petroleum ether=1:12) that the raw material CPD070-5 was completely consumed.

After cooling to room temperature. The reaction solution was washed with deionized water (50 mL*3), and successively subjected to liquid separation, concentration, purification by silica gel column chromatography (200-300 mesh silicagel, acetate:petroleum ether=1:25 as an eluent) and concentration, to obtain CPD070-6 as a light yellow liquid (3.94 g, purity: 99.58%, yield: 90.410). Mass spectrum: 167.03 (M+H).

### Synthesis of Compound CPD070-7

CPD070-6 (18.00 g, 108.35 mmol), potassium peroxymonosulfonate (85.25 g, 238.37 mmol), ammonium bromide (23.35 g, 238.37 mmol), acetonitrile (200 mL), and deionized water (200 mL) were added to a 1000 mL three-necked round-bottom flask, and the flask was displaced with nitrogen three times. The resulting mixture was stirred at room temperature for 6 h. TLC monitored (ethyl acetate:petroleum ether=1:8) that the raw material CPD070-6 was completely consumed.

The reaction solution was concentrated to remove the solvent, added with dichloromethane (500 mL), washed with deionized water (150 mL*3), and successively subjected to liquid separation, concentration, purification by silica gel column chromatography (200-300 mesh silica gel, acetate:petroleum ether=1:30 as an eluent), and concentration, to obtain CPD070-7 as a light yellow liquid (24.40 g, purity: 99.61%, yield: 85.62%). Mass spectrum: 263.06 (M+H).

### Synthesis of Compound CPD070-8

CPD070-7 (15.00 g, 57.03 mmol), triphenylphosphine (17.95 g, 68.44 mmol), diethyl azodicarboxylate (11.92 g, 68.44 mmol), and dry THF (150 mL) were added to a 500 mL three-necked round-bottom flask, and the flask was displaced with nitrogen three times. The resulting mixture was stirred at room temperature for 24 h. TLC monitored (ethyl acetate:petroleum ether 1:15) that the raw, material CPD070-7 was completely consumed.

The reaction solution was concentrated to remove the solvent, added with dichloromethane (700 mL), washed with deionized water (200 mL*3), and successively subjected to liquid separation, concentration, purification by silica gel column chromatography (200-300 mesh silica gel, acetate:petroleum ether=1:35 as an eluent), and concentration, to obtain CPD070-8 as a light yellow solid (18.02 g, purity: 99.59%, yield: 62.19%), Mass spectrum: 507.20 (M+H).

### Synthesis of Compound CPD070-9

CPD070-8 (16.00 g, 31.49 mmol) and glacial acetic acid (160 mL) were added to a 1000 mL three-necked round-bottom flask, followed by the dropwise addition of concentrated nitric acid (160 mL), and the mixture was stirred at room temperature for 24 h. TLC monitored (ethyl acetate:petroleum ether=1:6) that the raw material CPD070-8 was completely consumed.

A light yellow solid directly precipitated out from the reaction solution. After suction filtration, deionized water (500 mL) was added to the filter cake, and the mixture was pulped at room temperature for 1 hour. After suction filtration, the filter cake was washed with deionized water (100 mL) and with iced methanol (100 mL) and dried in vacuum at 50° C. to obtain Compound CPD070-9 as a light yellow solid (12.93 g, purity: 99.52%, yield: 82.14%). Mass spectrum: 499.24 (M+H).

### Synthesis of Compound CPD070-10

CPD070-9 (10.00 g, 20.00 mmol), CPD070-6 (3.32 g, 20.00 mmol), dichlorodi-tert-butyl-(4-dimethylaminophenyl)phosphine palladium(II) (284.04 mg, 0.40 mmol), sodium carbonate (4.24 g, 40 mmol), toluene (150 mL), ethanol (50 mL), and deionized water (50 mL) were added to a 500 mL three-necked round-bottom flask, and the flask was displaced with nitrogen three times. Subsequently, the reaction system was heated to 70° C. and reacted for 5 hours. TLC monitored (methanol:dichloromethane=1:20) that the raw material CPD070-9 was completely consumed.

A yellow solid directly precipitated out from the reaction solution. After suction filtration, deionized water (500 mL) was added to the filter cake, and the mixture was pulped at room temperature for 1 hour After suction filtration, the filter cake was washed with deionized water (100 mL) and with iced methanol (80 mL) and dissolved with 10-fold chloroform. After hot filtration over silica gel (20 g, 200-300 mesh silica gel) and concentration, recrystallization was then carried out twice with 5-fold chloroform. After driving in vacuum at 50° C., Compound CPD070-9 as a yellow solid was obtained (7.70 g, purity: 99.91%, yield: 76.32%). Mass spectrum: 505.22 (M+H).

### Synthesis of Compound CPD070

Referring to the synthesis and purification method for Compound CPD001, only by changing the corresponding raw materials, target Compound CPD070 as a yellowish brown solid (4.43 g. purity: 99.91%, yield: 61.80%) was obtained. 4.43 g of crude CPD070 was sublimated and purified to obtain sublimated pure CPD070 (2.01 g, purity: 99.91%, yield: 45.37%). Mass spectrum: 503.16 (M+H).

¹³C NMR (100 MHz, CDCl₃) δ 147.72, 144.12, 143.07, 124.68, 113.35, 111.90, 92.09, 90.60.

¹⁹F NMR (377 MHz, CDCl₃) δ −117.90, −146.20.

### Synthesis of Compound CPD085

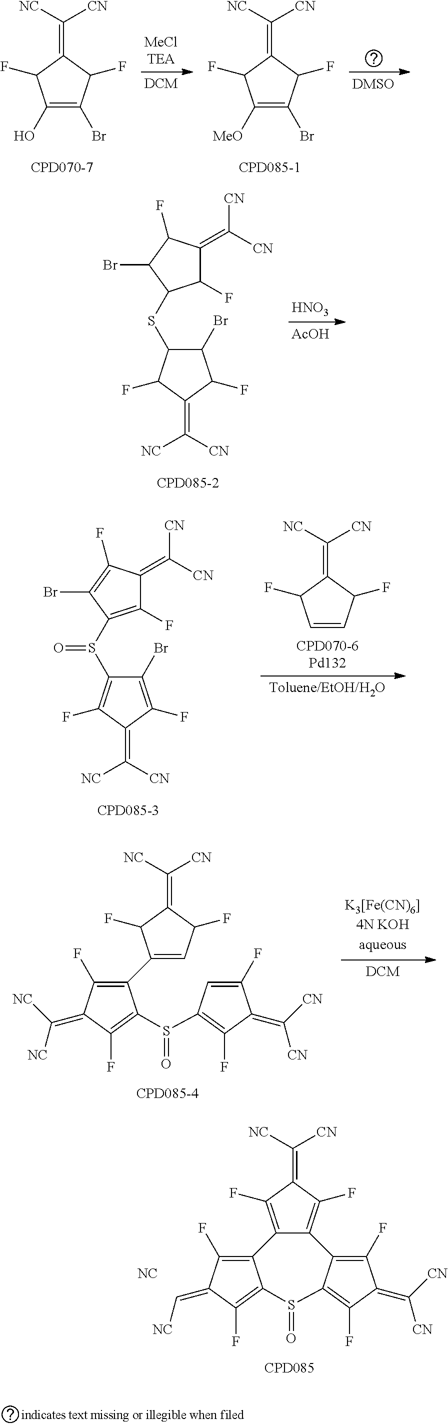

### Synthesis of Compound CPD085-1

CPD070-7 (16.00 g, 60.83 mmol), triethylamine (12.31 g, 121.66 mmol) and dichloromethane (160 mL) were added to a 500 mL three-necked round-bottom flask, and the flask was displaced with nitrogen three times. Subsequently, the reaction system was cooled to 0° C. methylsulfonyl chloride (10.45 g, 91.24 mmol) was dropwise added, and the reaction system was maintained at this temperature and stirred for 1 hour. TLC monitored (ethyl acetate:petroleum ether=1:20) that the raw material CPD070-7 was completely consumed.

The reaction solution was washed by adding deionized water (60 mL*3) and successively subjected to concentration, purification by silica gel column chromatography (200-300 mesh silica gel, acetate:petroleum ether=1:30 as an eluent), and concentration, to obtain CPD085-1 as a light yellow liquid (19.72 g, purity: 99.68%, yield: 95.04%). Mass spectrum: 341.03 (M+H).

### Synthesis of Compound CPD085-2

CPD085-1 (17.85 g, 52.33 mmol), sodium sulfide nonahydrate (25.13 g, 261.65 mmol), and N,N-dimethylformamide (180 mL) were added to a 500 mL three-necked round-bottom flask, and the flask was displaced with nitrogen three times. Subsequently, the reaction system was heated to 50° C. and reacted overnight. TLC monitored (ethyl acetate:petroleum ether=1:20) that the raw material CPD085-1 was completely consumed.

The reaction solution was concentrated to remove the solvent, added dichloromethane (500 mL), washed by adding deionized water (150 mL*3), and successively subjected to concentration, purification by silica gel column chromatography (200-300 mesh silica gel, acetate:petroleum ether=1:25 as an eluent), and concentration, to obtain CPD085-2 as a light yellow liquid (15.45 g, purity: 99.56%, yield: 56.36%). Mass spectrum: 523.08 (M+H).

### Synthesis of Compound CPD085-3

Referring to the synthesis and purification method for Compound CPD070-9, only by changing the corresponding raw materials, target Compound CPD085-3 as a light yellow solid (11.76 g, purity: 99.51%, yield: 8615%) was obtained. Mass spectrum: 531.02 (M+H).

### Synthesis of Compound CPD085-4

Referring to the synthesis and purification method for Compound CPD070-10, only by changing the corresponding raw materials, target Compound CPD085-4 as a yellow solid (6.88 g, purity: 99.90%, yield: 75.11%) was obtained. Mass spectrum: 537.06 (M+H).

### Synthesis of Compound CPD085

Referring to the synthesis and purification method for Compound CPD001, only by changing the corresponding raw materials, target Compound CPD085 as a yellowish brown solid (4.05 g, purity: 99.91%, yield: 62.33%) was obtained. 4.05 g of crude CPD085 was sublimated and purified to obtain sublimated pure CPD085 (1.88 g, purity: 99.91%, yield: 46.41%). Mass spectrum: 535.06 (M+H).

¹³C NMR (100 MHz, CDCl₃) δ 144.12, 142.16, 126.14, 113.35, 112.40, 97.72, 92.08.

¹⁹F NMR (377 MHz, CDCl₃) δ −146.12.

### Synthesis of Compound CPD094

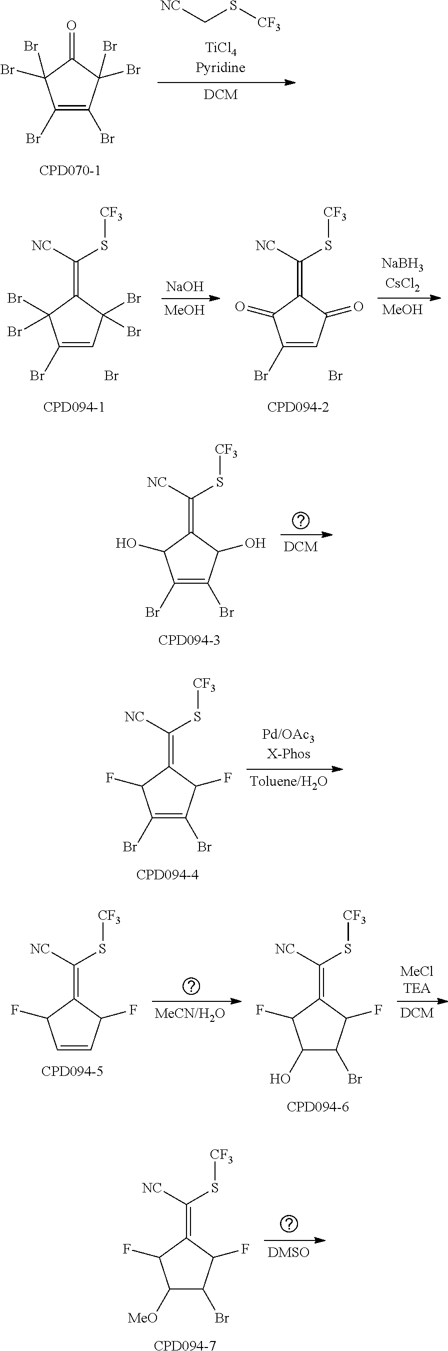

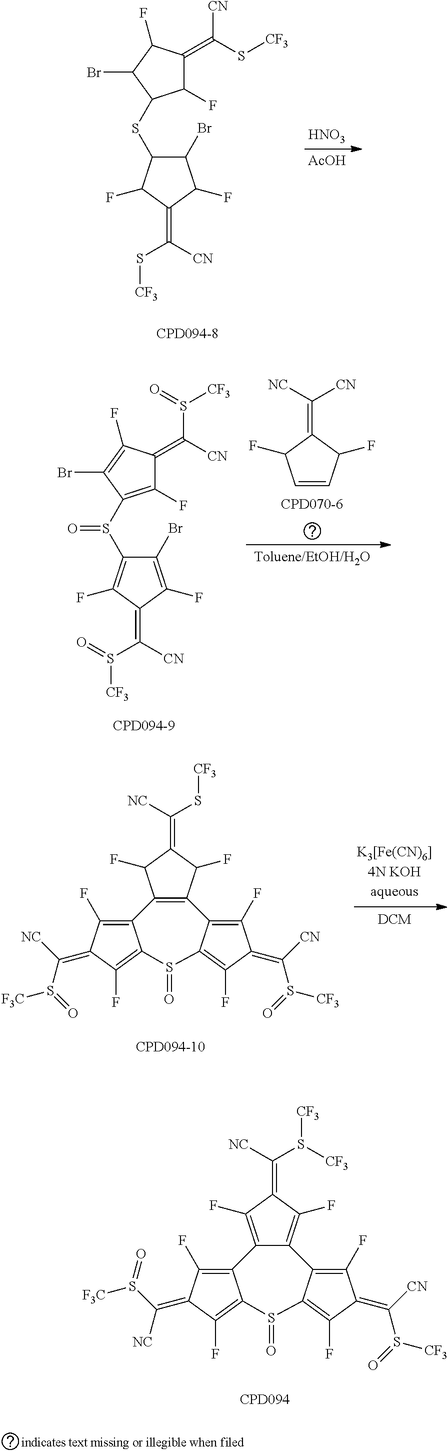

### Synthesis of Compound CPD094-1

Referring to the synthesis and purification method for Compound CPD070-2, only by changing the corresponding raw materials, target Compound CPD094-1 as a light yellow liquid (35.12 g, purity: 99.56%, yield: 57.68%) was obtained. Mass spectrum: 673.28 (M+H).

### Synthesis of Compound CPD094-2

Referring to the synthesis and purification method for Compound CPD070-3, only by changing the corresponding raw materials, target Compound CPD094-2 as a light yellow liquid (16.73 g, purity: 99.68%, yield: 83.98%) was obtained, Mass spectrum: 390.21 (M+H).

### Synthesis of Compound CPD094-3

Referring to the synthesis and purification method for Compound CPD070-4, only by changing the corresponding raw materials, target Compound CPD094-3 as a light yellow liquid (15.55 g, purity: 99.62%/n, yield: 93.34%) was obtained. Mass spectrum: 394.03 (M+H).

### Synthesis of Compound CPD094-4

Referring to the synthesis and purification method for Compound CPD070-5, only by changing the corresponding raw materials, target Compound CPD094-4 as a light yellow liquid (13.10 g, purity: 99.50%, yield: 75.17%) was obtained. Mass spectrum: 398.02 (M+14H).

### Synthesis of Compound CPD094-5

Referring to the synthesis and purification method for Compound CPD070-6, only by changing the corresponding raw materials, target Compound CPD094-5 as a light yellow liquid (8.94 g, purity: 99.53%, yield: 88.78%) was obtained. Mass spectrum: 242.04 (M+H).

### Synthesis of Compound CPD094-6

Referring to the synthesis and purification method for Compound CPD070-7, only by changing the corresponding raw materials, target Compound CPD094-6 as a light yellow liquid (18.98 g, purity: 99.63%, yield: 87.36%) was obtained. Mass spectrum: 338.12 (M+H).

### Synthesis of Compound CPD094-7

Referring to the synthesis and purification method for Compound CPD085-1, only by changing the corresponding raw materials, target Compound CPD094-7 as a light yellow liquid (20.05 g, purity: 99.67%, yield: 95.34%) was obtained. Mass spectrum: 416.00 (M+H).

### Synthesis of Compound CPD094-8

Referring to the synthesis and purification method for Compound CPD085-2, only by changing the corresponding raw materials, target Compound CPD094-8 as a light yellow liquid (16.33 g, purity: 99.61%, yield: 57.33%) was obtained. Mass spectrum: 673.01 (M+H).

### Synthesis of Compound CPD094-9

Referring to the synthesis and purification method for Compound CPD070-9, only by changing the corresponding raw materials, target Compound CPD094-9 as a light yellow solid (14.35 g, purity: 99.58%, yield: 84.10%) was obtained Mass spectrum: 713.14 (M+H).

### Synthesis of Compound CPD094-10

Referring to the synthesis and purification method for Compound CPD070-10, only by changing the corresponding raw materials, target Compound CPD094-10 as a light yellow solid (6.05 g, purity: 99.92%, yield: 61.11%) was obtained. Mass spectrum: 794.26 (M+1).

### Synthesis of Compound CPD094

Referring to the synthesis and purification method for Compound CPD001, only by changing the corresponding raw materials, target Compound CPD094 as a yellowish brown solid (5.55 g, purity: 99.92% a, yield: 58.74%) was obtained. 5.55 g of crude CPD094 was sublimated and purified to obtain sublimated pure CPD094 (2.35 g, purity: 99.92%, yield: 42.34%). Mass spectrum: 808.06 (M+H).

¹³C NMR (100 MHz, CDCl₃) δ 201.83, 196.96, 123.85, 109.77, 107.98.

¹⁹F NMR (377 MHz, CDCl₃) δ −77.9, −146.21.

### Example of Use: Manufacturing of Organic Electroluminescent Device

A 50 mm*50 mm*1.0 mm glass substrate with an ITO (100 nm) anode electrode was ultrasonically cleaned in ethanol for 10 minutes, then dried at 150° C. and then treated with N₂ Plasma for 30 minutes. The washed glass substrate was arranged on a substrate holder of a vacuum evaporation device. Firstly, compound HTM1 and P-dopant (at a ratio of 97%:3%, P-dopant was Comparative Compound X or a compound of the present disclosure) were evaporated on the side of the glass substrate, on which there was an anode electrode wire, by covering the electrode in a co-evaporation manner to form a thin film with a thickness of 10 nm, followed by immediately evaporation of a layer of HTM1 to form a thin film with a thickness of about 60 nu, and then evaporation of a layer of HTM2 on the HTM1 thin film to form a thin film with a thickness of about 10 nm. Then, Host Material 1, Host Material 2, and a doping compound (RD) were evaporated on the HTM2 film layer by means of co-evaporation again to form a film with a thickness of 40 nm. ETL:LiQ (35 nm, the ratio was 50%:50%) was evaporated on the luminescent layer by means of co-evaporation. Yb (1 nm) was then evaporated on the electron transport layer material, and finally a layer of metal Ag (15 nm) was evaporated as an electrode.

| | | | | | Electron |
|---|---|---|---|---|---|
| | | | | | transport |
| Example | HIL | HTL | EBL | Luminescent layer | layer |
| A1 | HTM1:CPD 001 | HTM1 | HTM2 | H1:H2:RD | ETL:LiQ |
| A2 | HTM1:CPD 003 | HTM1 | HTM2 | H1:H2:RD | ETL:LiQ |
| A3 | HTM1:CPD 016 | HTM1 | HTM2 | H1:H2:RD | ETL:LiQ |
| A4 | HTM1:CPD 031 | HTM1 | HTM2 | H1:H2:RD | ETL:LiQ |
| A5 | HTM1:CPD 032 | HTM1 | HTM2 | H1:H2:RD | ETL:LiQ |
| A6 | HTM1:CPD 046 | HTM1 | HTM2 | H1:H2:RD | ETL:LiQ |
| A7 | HTM1:CPD 058 | HTM1 | HTM2 | H1:H2:RD | ETL:LiQ |
| A8 | HTM1:CPD 070 | HTM1 | HTM2 | H1:H2:RD | ETL:LiQ |
| A9 | HTM1:CPD 085 | HTM1 | HTM2 | H1:H2:RD | ETL:LiQ |
| A10 | HTM1:CPD 094 | HTM1 | HTM2 | H1:H2:RD | ETL:LiQ |
| Comparative | HTM1: Comparative | HTM1 | HTM2 | H1:H2:RD | ETL:LiQ |
| Example 1 | Compound 1 | | | | |
| Comparative | HTMI: Comparative | HTM1 | HTM2 | H1:H2:RD | ETL:LiQ |
| Example 2 | Compound 2 | | | | |
| Comparative | HTM1: Comparative | HTM1 | HTM2 | H1:H2:RD | ETL:LiQ |
| Example 3 | Compound 3 | | | | |

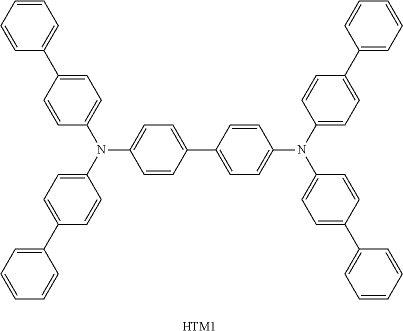

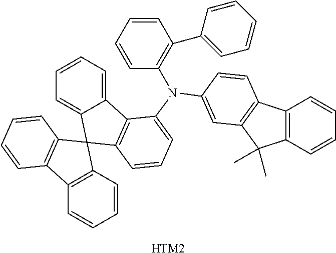

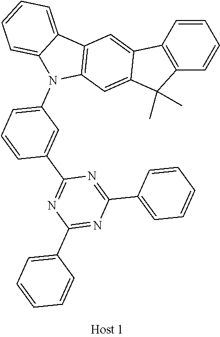 [SMILES: CC1(C)c2ccccc2-c2cc3c4ccccc4n(-c4cccc(-c5nc(-c6ccccc6)nc(-c6ccccc6)n5)c4)c3cc21]

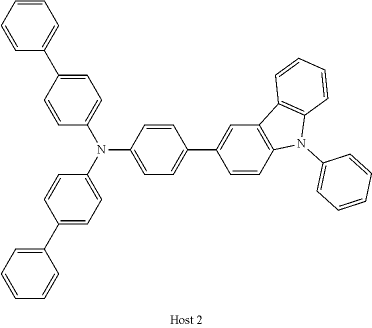 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

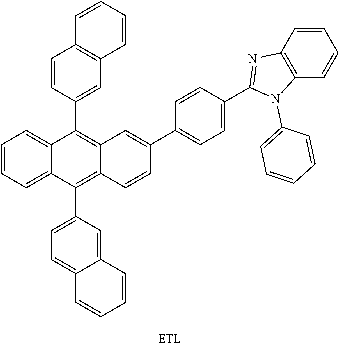 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

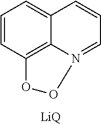

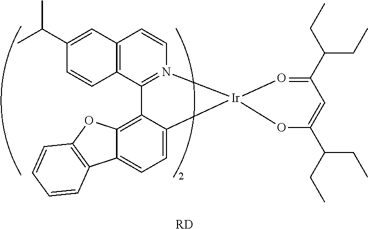

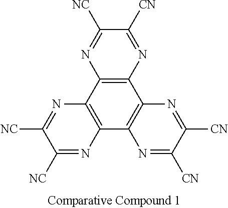 [SMILES: N#Cc1nc2c3nc(C#N)c(C#N)nc3c3nc(C#N)c(C#N)nc3c2nc1C#N]

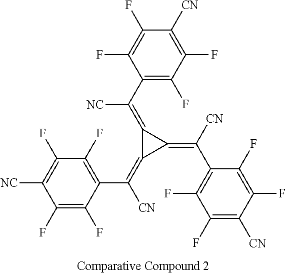 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

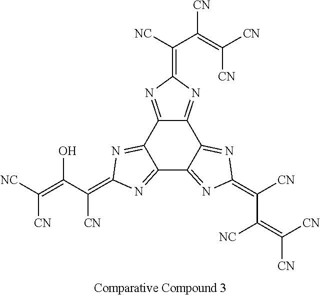 [SMILES: N#CC(C#N)=C(O)/C(C#N)=C1\N=c2c(c3c(c4c2=N/C(=C(\C#N)C(C#N)=C(C#N)C#N)N=4)=N/C(=C(\C#N)C(C#N)=C(C#N)C#N)N=3)=N1]

Evaluation: The above-mentioned devices were tested for device performance. In each of the examples and comparative examples, by using a constant current power supply (Keithley 2400), using a fixed current, density, that flowed through the luminescent element, and using a spectral radiance luminance meter (CS 2000), luminescence spectrum was tested. In addition, the voltage value was measured, and the time when the test brightness was 95%) of the initial brightness (LT915) was measured. The results were as follows: the current efficiency and device lifetime were both calculated based on the value of Comparative Compound 1 as 100%.

| | Starting | | | |
|---|---|---|---|---|
| | voltage @ | Current | Color | |
| | 20 mA/cm² | efficiency | coordinates | LT95@ |
| | V | @20 mA/cm² | (CIE X, Y) | 5000 nits |
| Example A1 | 3.87 | 109 | 0.68, 0.32 | 121 |
| Example A2 | 3.89 | 112 | 0.68, 0.32 | 126 |
| Example A3 | 3.91 | 116 | 0.68, 0.32 | 115 |
| Example A4 | 3.88 | 110 | 0.68, 0.32 | 119 |
| Example A5 | 3.93 | 113 | 0.68, 0.32 | 123 |
| Example A6 | 3.95 | 115 | 0.68, 0.32 | 121 |
| Example A7 | 4.01 | 109 | 0.68, 0.32 | 115 |
| Example A8 | 4.04 | 118 | 0.68, 0.32 | 118 |
| Example A9 | 3.98 | 115 | 0.68, 0.32 | 125 |
| Example A10 | 3.97 | 119 | 0.68, 0.32 | 122 |
| Comparative | 4.38 | 100 | 0.68, 0.32 | 100 |
| Example 1 | | | | |
| Comparative | 4.15 | 107 | 0.68, 0.32 | 113 |
| Example 2 | | | | |
| Comparative | 4.12 | 102 | 0.68, 0.32 | 94 |
| Example 3 | | | | |

From the comparison of the data in the above table, it can be seen that among devices with the same color coordinates, the organic electroluminescent devices in which the compound of the present disclosure is used as a P-type dopant all exhibit superior performance in terms of driving voltage, luminous efficiency and device lifetime as compared with Comparative Compounds 1, 2 and 3.

LUMO energy level test: The electrochemical properties of the compound were determined by cyclic voltammetry (CV). Model CS300 electrochemical workstation produced by Corrtest Instrument Corp., Ltd., Wuhan, China was used in the test, and a three-electrode working system was used, wherein a platinum disk electrode was used as a working electrode, an Ag/AgCl saturated KCl electrode was used as a reference electrode, and a platinum wire electrode was used as an auxiliary electrode. With anhydrous N,N-Dimethylformamide (DMF) as a solvent and 0.1 mol/L tetrabutylammonium hexafluorophosphate as a supporting electrolyte, the compound to be tested was prepared into a 10⁻³ mol/L solution, and nitrogen was introduced into the solution for 10 min for removal of oxygen before testing. Instrument parameter settings: The scanning rate was 100 mV/s, and ferrocene was used for potential calibration, wherein the absolute potential of ferrocene under vacuum condition was set to −4.8 eV. The corresponding calculation formula was as follows: LUMO=−[E^{red} (Sample)−E(Fc/Fc⁺)+4.8] eV. The LUMO energy levels of the compounds of the present disclosure and the comparative compounds were tested and calculated.

| Material | LUMO |
|---|---|
| CPD 001 | −5.10 |
| CPD 003 | −5.04 |
| CPD 016 | −5.11 |
| CPD 031 | −5.15 |
| CPD 032 | −5.08 |
| CPD 046 | −5.13 |
| CPD 058 | −5.09 |
| CPD 070 | −5.04 |
| CPD 085 | −5.05 |
| CPD 094 | −5.07 |
| Comparative Compound 1 | −4.44 |
| Comparative Compound 2 | −5.02 |
| Comparative Compound 3 | −5.17 |

From the comparison of the data in the above table, it can be seen that the compounds of the present disclosure have a lower LUMO energy level (<−5.0 eV), which can form a good match with the HOMO energy level of the hole transport material, and can effectively form holes, increase the hole concentration, improve the hole injection and transmission efficiency, and finally reduce the working voltage of a device to improve the luminous efficiency.

By means of a special collocation of substituents in the present disclosure, compared with the prior art, the compound of the present disclosure has a relatively low LUMO energy level, and the prepared red-light device has a low driving voltage, a better device luminous efficiency, and an improved lifetime. The above results indicate that the compound of the present disclosure can be used in an organic electroluminescent device as a hole injection layer material and has the potential to be applied to the OLED industry.

1. An organic compound having a structure shown in formula (1), 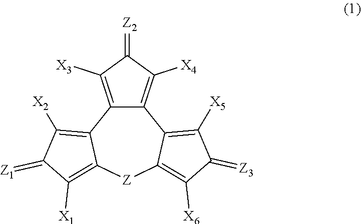
wherein Z is a single bond, O, S, SO or SO₂;
X₁-X₆ are each independently X, X-substituted C6-C30 aryl or X-substituted C2-C30 heteroaryl, and X is F, CN, or completely or partially fluorine-substituted C1-C4 fluoroalkyl;
Z₁-Z₃ are each independently O, S, Se, NR₁, or CR₂R₃;
wherein R₁-R₃ are each independently hydrogen, deuterium, halogen, CN, substituted or unsubstituted C1-C20 alkyl, substituted or unsubstituted C3-C20 cycloalkyl, substituted or unsubstituted C1-C20 heteroalkyl, substituted or unsubstituted C7-C30 aralkyl, substituted or unsubstituted C1-C20 alkoxy, substituted or unsubstituted C6-C30 aryloxy, substituted or unsubstituted C2-C20 alkenyl, substituted or unsubstituted C2-C20 alkynyl, substituted or unsubstituted C6-C30 aryl, substituted or unsubstituted C3-C30 heteroaryl, substituted or unsubstituted C3-C20 alkylsilyl, substituted or unsubstituted C6-C20 arylsilyl, substituted or unsubstituted C1-C6 alkylsulfinyl, substituted or unsubstituted C1-C6 alkylsulfonyl, or substituted or unsubstituted C1-C6 alkylcarbonyl;
wherein at least one of R₁-R₃ is a group having an electron-withdrawing group;
wherein the substituents on R₁-R₃ are F, CN, completely or partially fluorine-substituted C1-C4 fluoroalkyl, completely or partially fluorine-substituted C1-C4 alkylsulfinyl, completely or partially fluorine-substituted C1-C4 alkylsulfonyl, or completely or partially fluorine-substituted C1-C4 alkylcarbonyl; and
wherein the heteroalkyl and heteroaryl contain at least one O, N or S heteroatom.

2. The organic compound according to claim 1, wherein Z is a single bond, O, or SO.

3. The organic compound according to claim 2, wherein Z is a single bond and has a structure shown in formula (2), 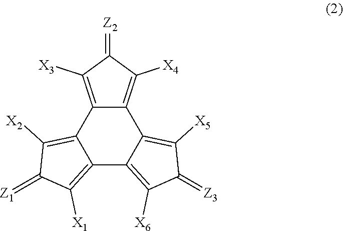
wherein X₁-X₆ are each independently X, X-substituted C6-C20 aryl or X-substituted C2-C10 heteroaryl, and X is F, CN, or completely or partially fluorine-substituted C1-C4 fluoroalkyl; and
Z₁-Z₃ are each independently O, S, NR₁, or CR₂R₃;
wherein R₁-R₃ are each independently selected from the group consisting of hydrogen, deuterium, halogen, CN, substituted or unsubstituted C1-C10 alkyl, substituted or unsubstituted C3-C10 cycloalkyl, substituted or unsubstituted C1-C10 heteroalkyl, substituted or unsubstituted C7-C20 aralkyl, substituted or unsubstituted C6-C20 aryl, substituted or unsubstituted C3-C20 heteroaryl, substituted or unsubstituted C1-C4 alkylsulfinyl, substituted or unsubstituted C1-C4 alkylsulfonyl, and substituted or unsubstituted C1-C4 alkylcarbonyl.

4. The organic compound according to claim 3, wherein at least two of Z₁-Z₃ are CR₂R₃.

5. The organic compound according to claim 4, wherein Z₁-Z₃ are all CR₂R₃.

6. The organic compound according to claim 5, wherein among these CR₂R₃, at least two are groups having an electron-withdrawing group.

7. The organic compound according to claim 6, wherein R₂ and R₃ are both groups having electron-withdrawing property.

8. The organic compound according to claim 7, wherein R₂-R₃ are each independently halogen, CN, substituted or unsubstituted C1-C6 alkyl, substituted or unsubstituted C7-C10 aralkyl, substituted or unsubstituted C6-C12 aryl, substituted or unsubstituted C3-C12 heteroaryl, substituted or unsubstituted C1-C4 alkylsulfinyl, substituted or unsubstituted C1-C4 alkylsulfonyl, or substituted or unsubstituted C1-C4 alkylcarbonyl;
and the substituents on R₂-R₃ are F, CN, CF₃, CF₃-sulfinyl, or CF₃-sulfonyl;
wherein X₁-X₆ are each independently X.

9. The organic compound according to claim 8, wherein the group having electron-withdrawing property is F, CN, CF₃, pyridine, pyrimidine, pyridazine, pyrazine, thiazole, oxazole, triazine, sulfinyl, sulfonyl, carbonyl, or C6-C12 aryl or C3-C12 heteroaryl containing one of the foregoing groups having electron-withdrawing property.

10. The organic compound according to claim 9, wherein X1-X6 are, F, N, or CF₃.

11. The organic compound according to claim 1, which has one of the following structures:

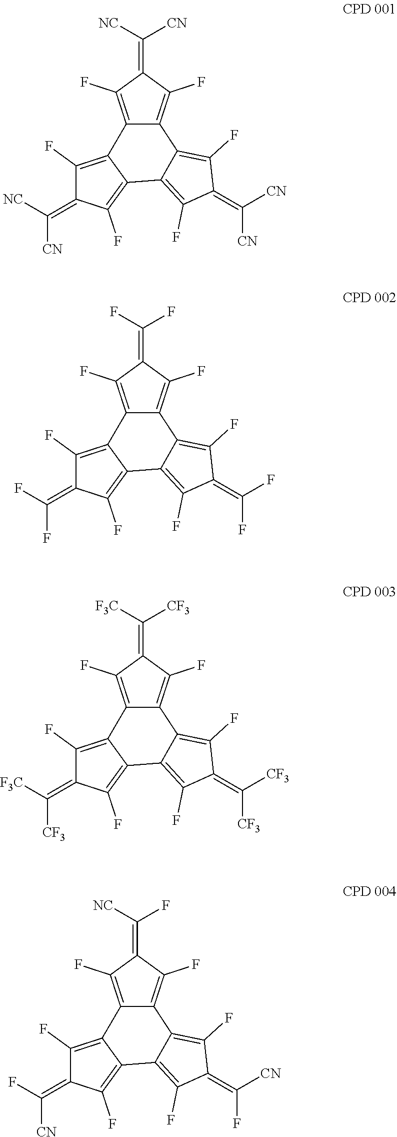 [SMILES: N#CC(C#N)=C1C(F)=c2c3c(c4c(c2=C1F)=C(F)C(=C(C#N)C#N)C=4F)=C(F)C(=C(C#N)C#N)C=3F ; FC(F)=C1C(F)=c2c3c(c4c(c2=C1F)=C(F)C(=C(F)F)C=4F)=C(F)C(=C(F)F)C=3F ; FC1=c2c3c(c4c(c2=C(F)C1=C(C(F)(F)F)C(F)(F)F)=C(F)C(=C(C(F)(F)F)C(F)(F)F)C=4F)=C(F)C(=C(C(F)(F)F)C(F)(F)F)C=3F ; N#CC(F)=C1C(F)=c2c3c(c4c(c2=C1F)=C(F)C(=C(F)C#N)C=4F)=C(F)C(=C(F)C#N)C=3F]

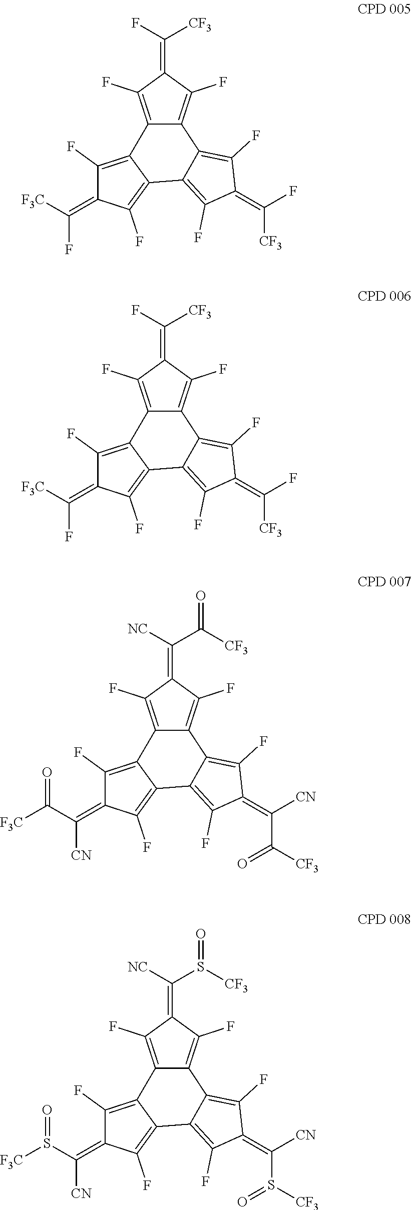 [SMILES: FC(=C1C(F)=c2c3c(c4c(c2=C1F)=C(F)C(=C(F)C(F)(F)F)C=4F)=C(F)C(=C(F)C(F)(F)F)C=3F)C(F)(F)F ; FC(=C1C(F)=c2c3c(c4c(c2=C1F)=C(F)C(=C(F)C(F)(F)F)C=4F)=C(F)C(=C(F)C(F)(F)F)C=3F)C(F)(F)F ; N#CC(C(=O)C(F)(F)F)=C1C(F)=c2c3c(c4c(c2=C1F)=C(F)C(=C(C#N)C(=O)C(F)(F)F)C=4F)=C(F)C(=C(C#N)C(=O)C(F)(F)F)C=3F ; N#CC(=C1C(F)=c2c3c(c4c(c2=C1F)=C(F)C(=C(C#N)S(=O)C(F)(F)F)C=4F)=C(F)C(=C(C#N)S(=O)C(F)(F)F)C=3F)S(=O)C(F)(F)F]

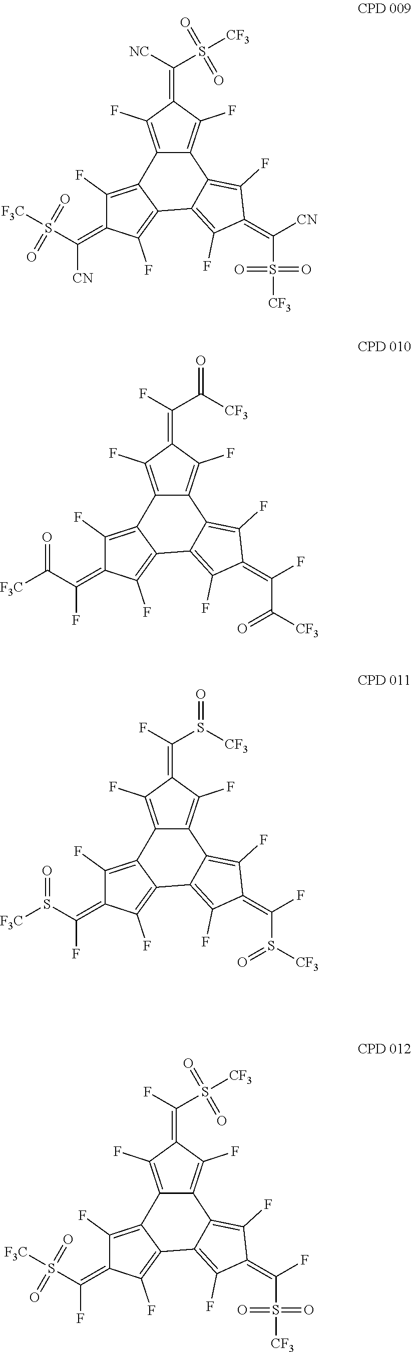 [SMILES: N#CC(=C1C(F)=c2c3c(c4c(c2=C1F)=C(F)C(=C(C#N)S(=O)(=O)C(F)(F)F)C=4F)=C(F)C(=C(C#N)S(=O)(=O)C(F)(F)F)C=3F)S(=O)(=O)C(F)(F)F ; O=C(C(F)=C1C(F)=c2c3c(c4c(c2=C1F)=C(F)C(=C(F)C(=O)C(F)(F)F)C=4F)=C(F)C(=C(F)C(=O)C(F)(F)F)C=3F)C(F)(F)F ; O=S(C(F)=C1C(F)=c2c3c(c4c(c2=C1F)=C(F)C(=C(F)S(=O)C(F)(F)F)C=4F)=C(F)C(=C(F)S(=O)C(F)(F)F)C=3F)C(F)(F)F ; O=S(=O)(C(F)=C1C(F)=c2c3c(c4c(c2=C1F)=C(F)C(=C(F)S(=O)(=O)C(F)(F)F)C=4F)=C(F)C(=C(F)S(=O)(=O)C(F)(F)F)C=3F)C(F)(F)F]

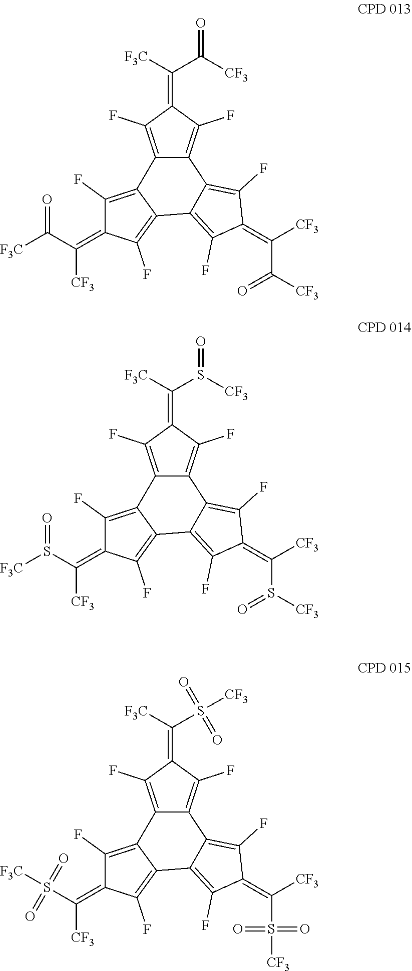 [SMILES: O=C(C(=C1C(F)=c2c3c(c4c(c2=C1F)=C(F)C(=C(C(=O)C(F)(F)F)C(F)(F)F)C=4F)=C(F)C(=C(C(=O)C(F)(F)F)C(F)(F)F)C=3F)C(F)(F)F)C(F)(F)F ; O=S(C(=C1C(F)=c2c3c(c4c(c2=C1F)=C(F)C(=C(S(=O)C(F)(F)F)C(F)(F)F)C=4F)=C(F)C(=C(S(=O)C(F)(F)F)C(F)(F)F)C=3F)C(F)(F)F)C(F)(F)F ; O=S(=O)(C(=C1C(F)=c2c3c(c4c(c2=C1F)=C(F)C(=C(C(F)(F)F)S(=O)(=O)C(F)(F)F)C=4F)=C(F)C(=C(C(F)(F)F)S(=O)(=O)C(F)(F)F)C=3F)C(F)(F)F)C(F)(F)F]

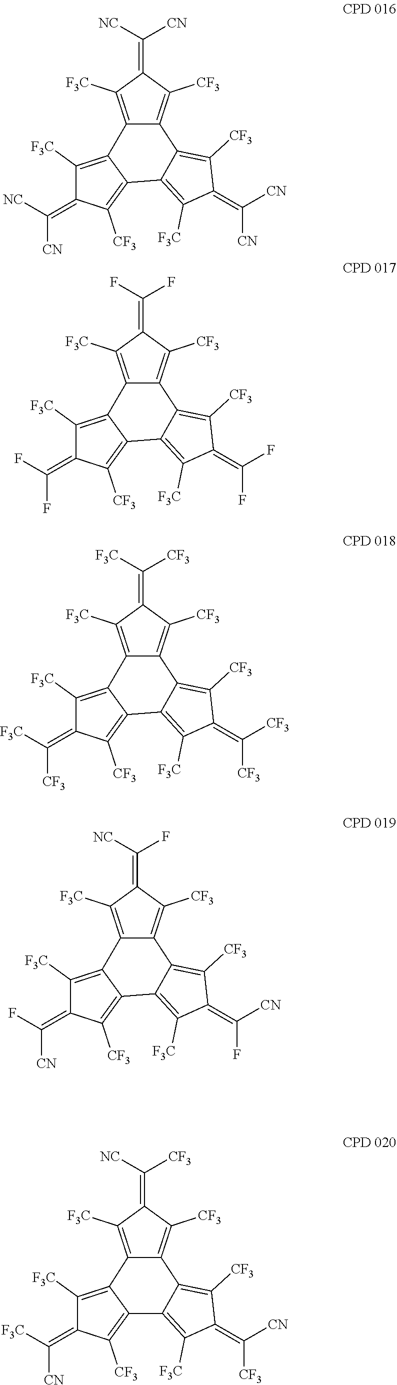

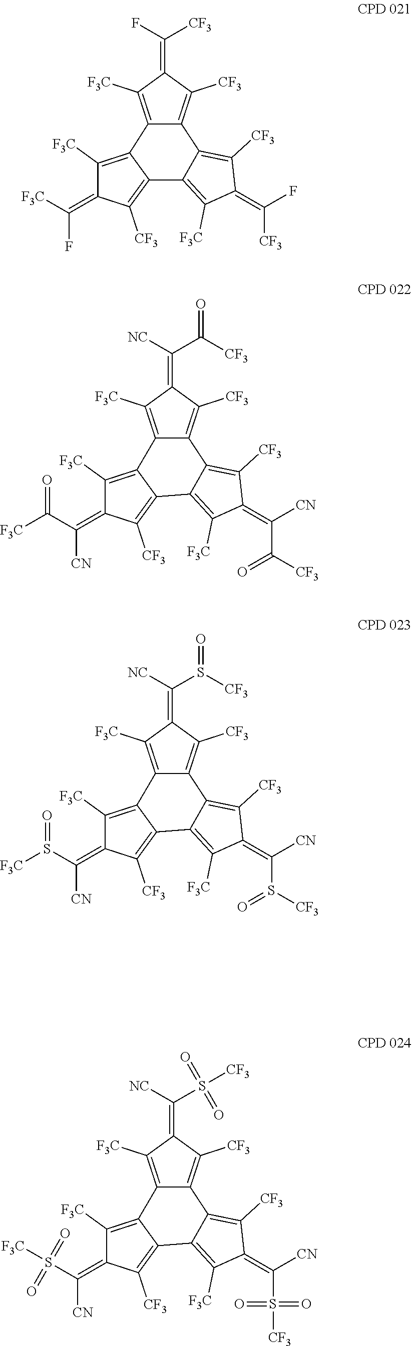

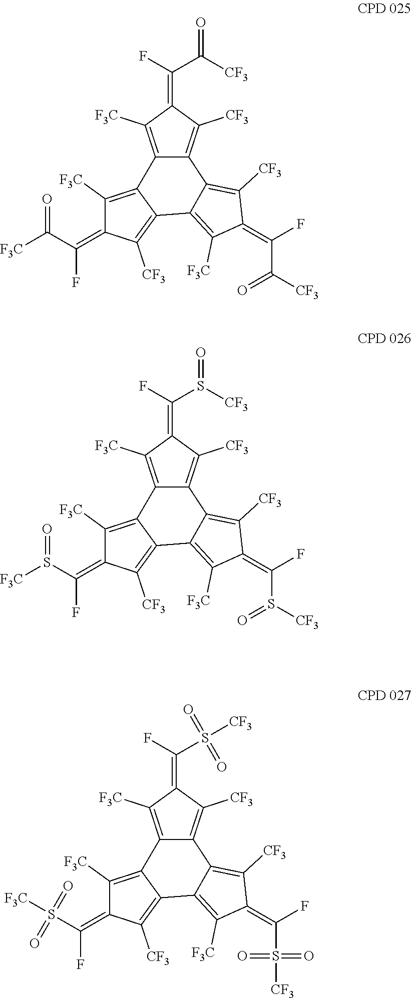 [SMILES: O=C(C(F)=C1C(C(F)(F)F)=c2c3c(c4c(c2=C1C(F)(F)F)=C(C(F)(F)F)C(=C(F)C(=O)C(F)(F)F)C=4C(F)(F)F)=C(C(F)(F)F)C(=C(F)C(=O)C(F)(F)F)C=3C(F)(F)F)C(F)(F)F ; O=S(C(F)=C1C(C(F)(F)F)=c2c3c(c4c(c2=C1C(F)(F)F)=C(C(F)(F)F)C(=C(F)S(=O)C(F)(F)F)C=4C(F)(F)F)=C(C(F)(F)F)C(=C(F)S(=O)C(F)(F)F)C=3C(F)(F)F)C(F)(F)F ; O=S(=O)(C(F)=C1C(C(F)(F)F)=c2c3c(c4c(c2=C1C(F)(F)F)=C(C(F)(F)F)C(=C(F)S(=O)(=O)C(F)(F)F)C=4C(F)(F)F)=C(C(F)(F)F)C(=C(F)S(=O)(=O)C(F)(F)F)C=3C(F)(F)F)C(F)(F)F]

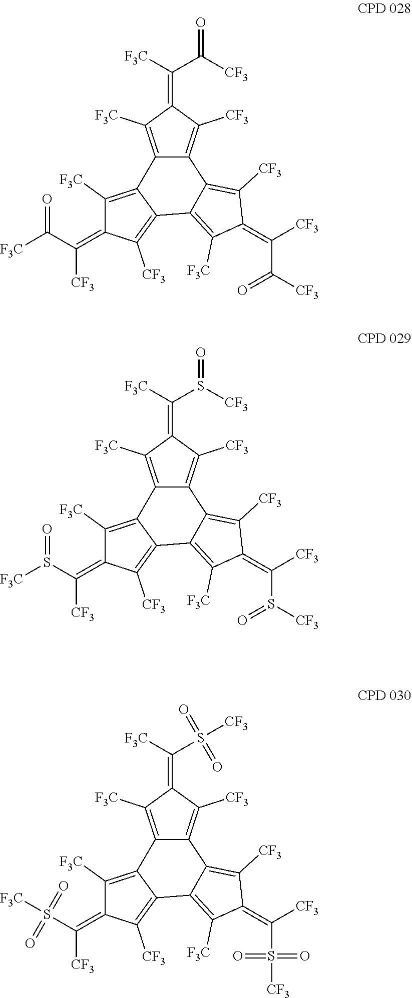

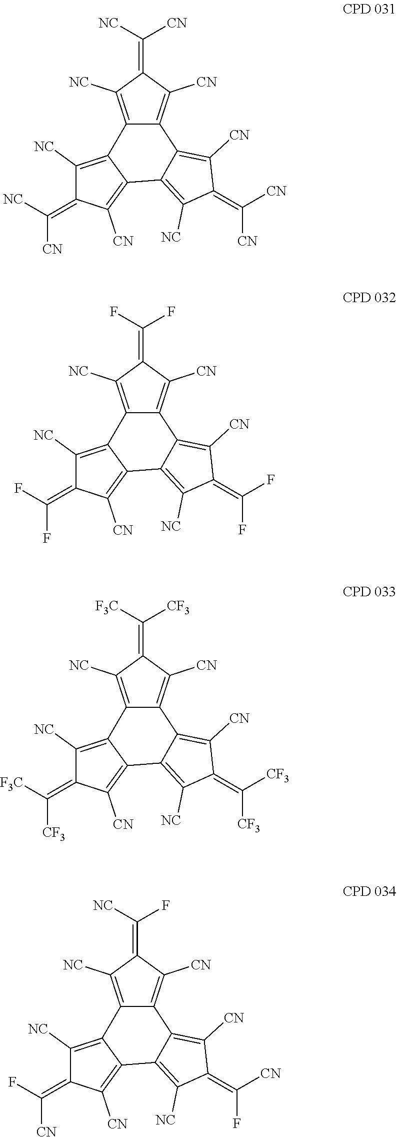 [SMILES: N#CC(C#N)=C1C(C#N)=c2c3c(c4c(c2=C1C#N)=C(C#N)C(=C(C#N)C#N)C=4C#N)=C(C#N)C(=C(C#N)C#N)C=3C#N ; N#CC1=c2c3c(c4c(c2=C(C#N)C1=C(F)F)=C(C#N)C(=C(F)F)C=4C#N)=C(C#N)C(=C(F)F)C=3C#N ; N#CC1=c2c3c(c4c(c2=C(C#N)C1=C(C(F)(F)F)C(F)(F)F)=C(C#N)C(=C(C(F)(F)F)C(F)(F)F)C=4C#N)=C(C#N)C(=C(C(F)(F)F)C(F)(F)F)C=3C#N ; N#CC(F)=C1C(C#N)=c2c3c(c4c(c2=C1C#N)=C(C#N)C(=C(F)C#N)C=4C#N)=C(C#N)C(=C(F)C#N)C=3C#N]

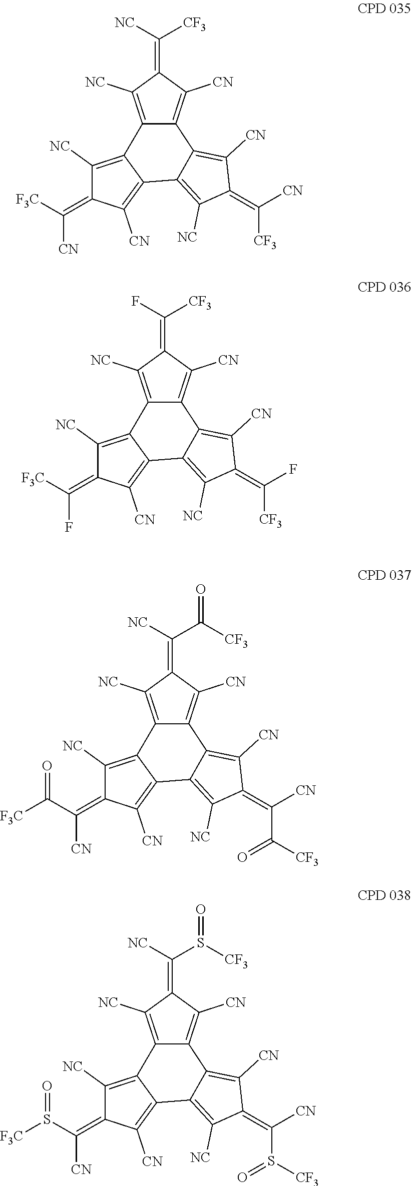 [SMILES: N#CC(=C1C(C#N)=c2c3c(c4c(c2=C1C#N)=C(C#N)C(=C(C#N)C(F)(F)F)C=4C#N)=C(C#N)C(=C(C#N)C(F)(F)F)C=3C#N)C(F)(F)F ; N#CC1=c2c3c(c4c(c2=C(C#N)C1=C(F)C(F)(F)F)=C(C#N)C(=C(F)C(F)(F)F)C=4C#N)=C(C#N)C(=C(F)C(F)(F)F)C=3C#N ; N#CC(C(=O)C(F)(F)F)=C1C(C#N)=c2c3c(c4c(c2=C1C#N)=C(C#N)C(=C(C#N)C(=O)C(F)(F)F)C=4C#N)=C(C#N)C(=C(C#N)C(=O)C(F)(F)F)C=3C#N ; N#CC(=C1C(C#N)=c2c3c(c4c(c2=C1C#N)=C(C#N)C(=C(C#N)S(=O)C(F)(F)F)C=4C#N)=C(C#N)C(=C(C#N)S(=O)C(F)(F)F)C=3C#N)S(=O)C(F)(F)F]

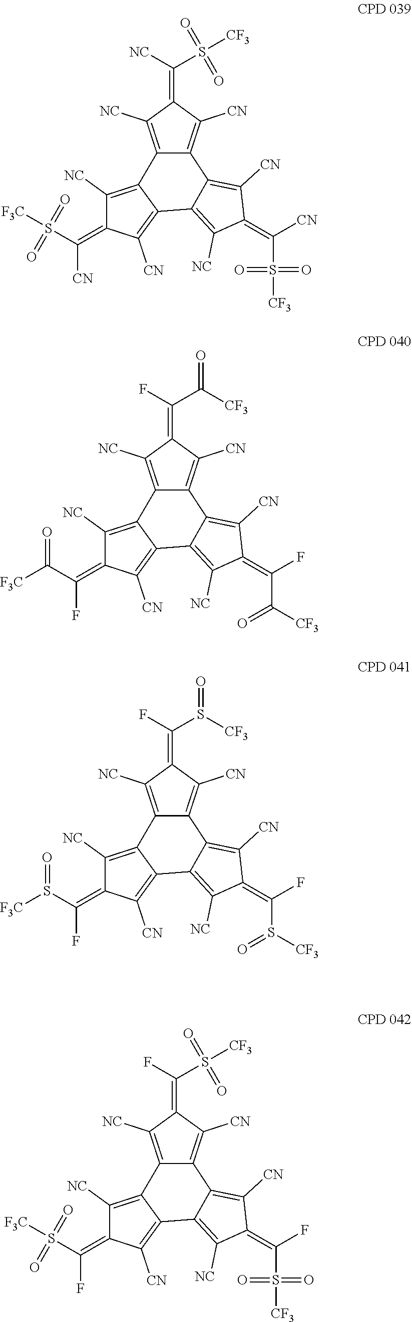 [SMILES: N#CC(=C1C(C#N)=c2c3c(c4c(c2=C1C#N)=C(C#N)C(=C(C#N)S(=O)(=O)C(F)(F)F)C=4C#N)=C(C#N)C(=C(C#N)S(=O)(=O)C(F)(F)F)C=3C#N)S(=O)(=O)C(F)(F)F ; N#CC1=c2c3c(c4c(c2=C(C#N)C1=C(F)C(=O)C(F)(F)F)=C(C#N)C(=C(F)C(=O)C(F)(F)F)C=4C#N)=C(C#N)C(=C(F)C(=O)C(F)(F)F)C=3C#N ; N#CC1=c2c3c(c4c(c2=C(C#N)C1=C(F)S(=O)C(F)(F)F)=C(C#N)C(=C(F)S(=O)C(F)(F)F)C=4C#N)=C(C#N)C(=C(F)S(=O)C(F)(F)F)C=3C#N ; N#CC1=c2c3c(c4c(c2=C(C#N)C1=C(F)S(=O)(=O)C(F)(F)F)=C(C#N)C(=C(F)S(=O)(=O)C(F)(F)F)C=4C#N)=C(C#N)C(=C(F)S(=O)(=O)C(F)(F)F)C=3C#N]

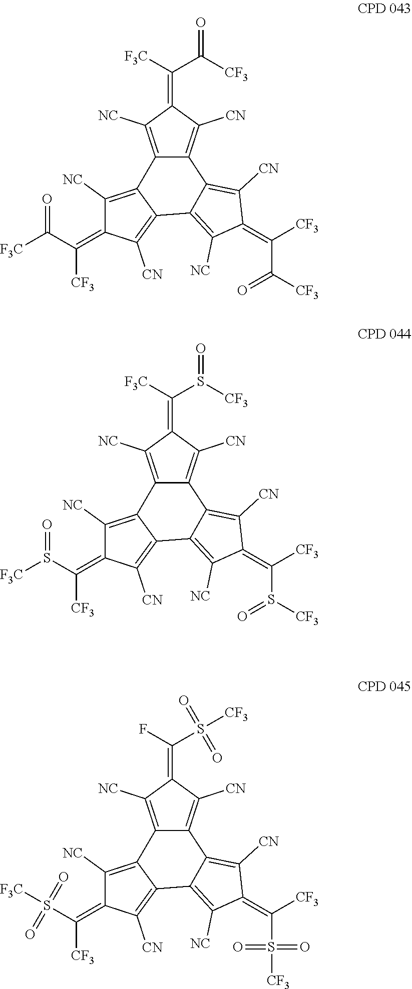 [SMILES: N#CC1=c2c3c(c4c(c2=C(C#N)C1=C(C(=O)C(F)(F)F)C(F)(F)F)=C(C#N)C(=C(C(=O)C(F)(F)F)C(F)(F)F)C=4C#N)=C(C#N)C(=C(C(=O)C(F)(F)F)C(F)(F)F)C=3C#N ; N#CC1=c2c3c(c4c(c2=C(C#N)C1=C(S(=O)C(F)(F)F)C(F)(F)F)=C(C#N)C(=C(S(=O)C(F)(F)F)C(F)(F)F)C=4C#N)=C(C#N)C(=C(S(=O)C(F)(F)F)C(F)(F)F)C=3C#N ; N#CC1=c2c(c3c(c4c2=C(C#N)/C(=C(\F)S(=O)(=O)C(F)(F)F)C=4C#N)=C(C#N)/C(=C(\C(F)(F)F)S(=O)(=O)C(F)(F)F)C=3C#N)=C(C#N)/C1=C(\C(F)(F)F)S(=O)(=O)C(F)(F)F]

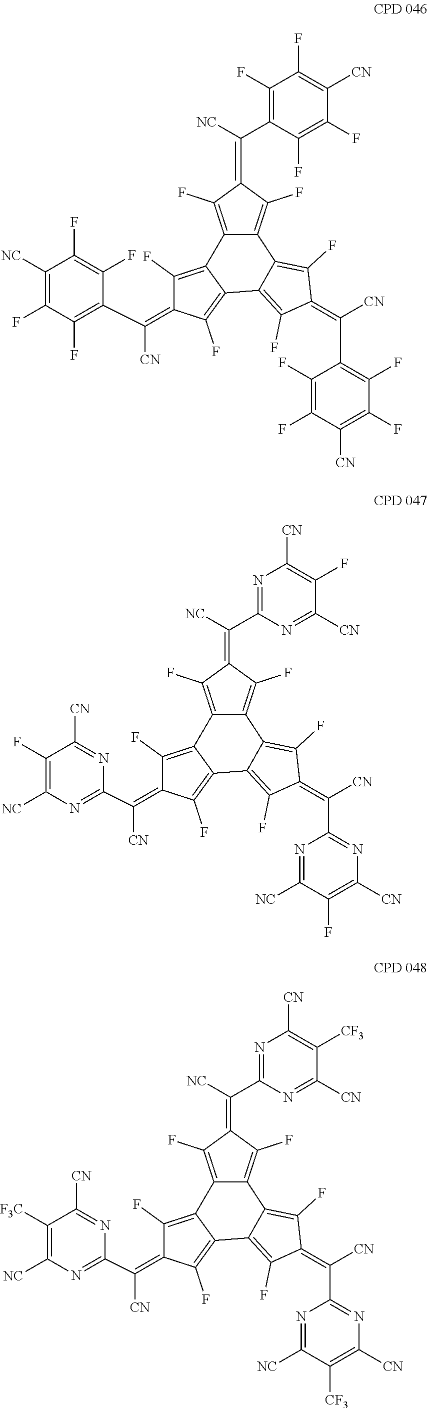 [SMILES: N#CC(=C1C(F)=c2c3c(c4c(c2=C1F)=C(F)C(=C(C#N)c1c(F)c(F)c(C#N)c(F)c1F)C=4F)=C(F)C(=C(C#N)c1c(F)c(F)c(C#N)c(F)c1F)C=3F)c1c(F)c(F)c(C#N)c(F)c1F ; N#CC(=C1C(F)=c2c3c(c4c(c2=C1F)=C(F)C(=C(C#N)c1nc(C#N)c(F)c(C#N)n1)C=4F)=C(F)C(=C(C#N)c1nc(C#N)c(F)c(C#N)n1)C=3F)c1nc(C#N)c(F)c(C#N)n1 ; N#CC(=C1C(F)=c2c3c(c4c(c2=C1F)=C(F)C(=C(C#N)c1nc(C#N)c(C(F)(F)F)c(C#N)n1)C=4F)=C(F)C(=C(C#N)c1nc(C#N)c(C(F)(F)F)c(C#N)n1)C=3F)c1nc(C#N)c(C(F)(F)F)c(C#N)n1]

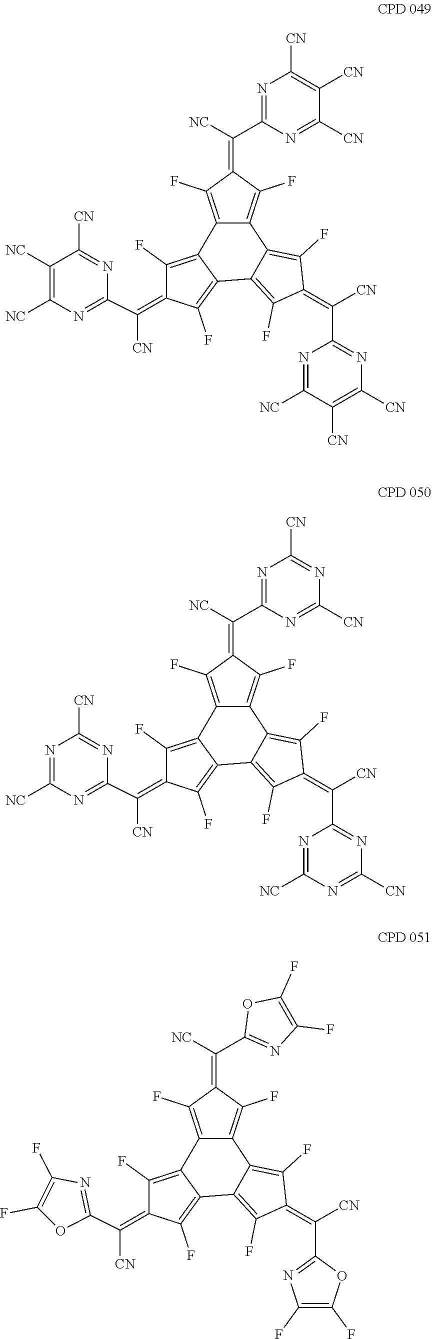 [SMILES: N#CC(=C1C(F)=c2c3c(c4c(c2=C1F)=C(F)C(=C(C#N)c1nc(C#N)c(C#N)c(C#N)n1)C=4F)=C(F)C(=C(C#N)c1nc(C#N)c(C#N)c(C#N)n1)C=3F)c1nc(C#N)c(C#N)c(C#N)n1 ; N#CC(=C1C(F)=c2c3c(c4c(c2=C1F)=C(F)C(=C(C#N)c1nc(C#N)nc(C#N)n1)C=4F)=C(F)C(=C(C#N)c1nc(C#N)nc(C#N)n1)C=3F)c1nc(C#N)nc(C#N)n1 ; N#CC(=C1C(F)=c2c3c(c4c(c2=C1F)=C(F)C(=C(C#N)c1nc(F)c(F)o1)C=4F)=C(F)C(=C(C#N)c1nc(F)c(F)o1)C=3F)c1nc(F)c(F)o1]

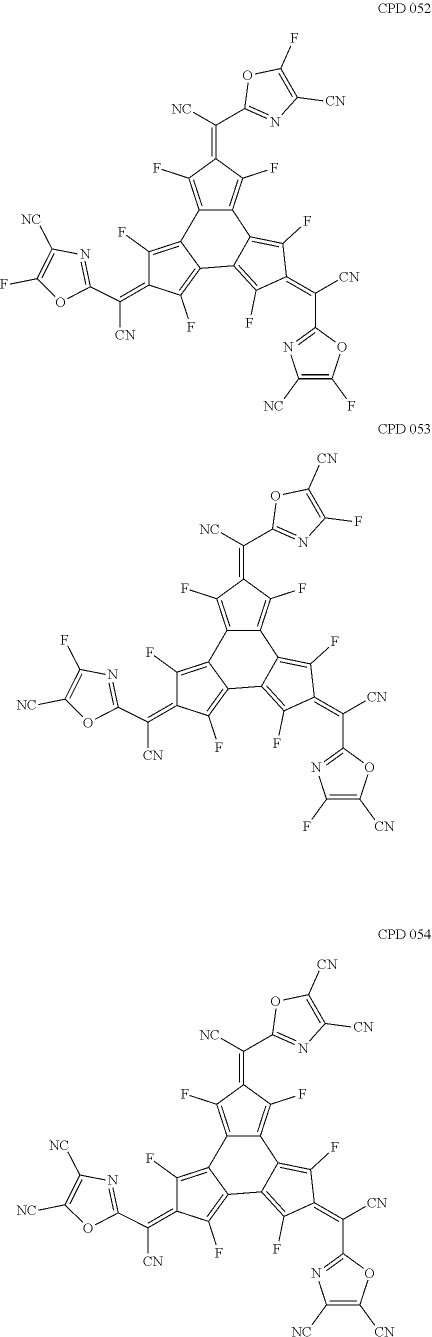 [SMILES: N#CC(=C1C(F)=c2c3c(c4c(c2=C1F)=C(F)C(=C(C#N)c1nc(C#N)c(F)o1)C=4F)=C(F)C(=C(C#N)c1nc(C#N)c(F)o1)C=3F)c1nc(C#N)c(F)o1 ; N#CC(=C1C(F)=c2c3c(c4c(c2=C1F)=C(F)C(=C(C#N)c1nc(F)c(C#N)o1)C=4F)=C(F)C(=C(C#N)c1nc(F)c(C#N)o1)C=3F)c1nc(F)c(C#N)o1 ; N#CC(=C1C(F)=c2c3c(c4c(c2=C1F)=C(F)C(=C(C#N)c1nc(C#N)c(C#N)o1)C=4F)=C(F)C(=C(C#N)c1nc(C#N)c(C#N)o1)C=3F)c1nc(C#N)c(C#N)o1]

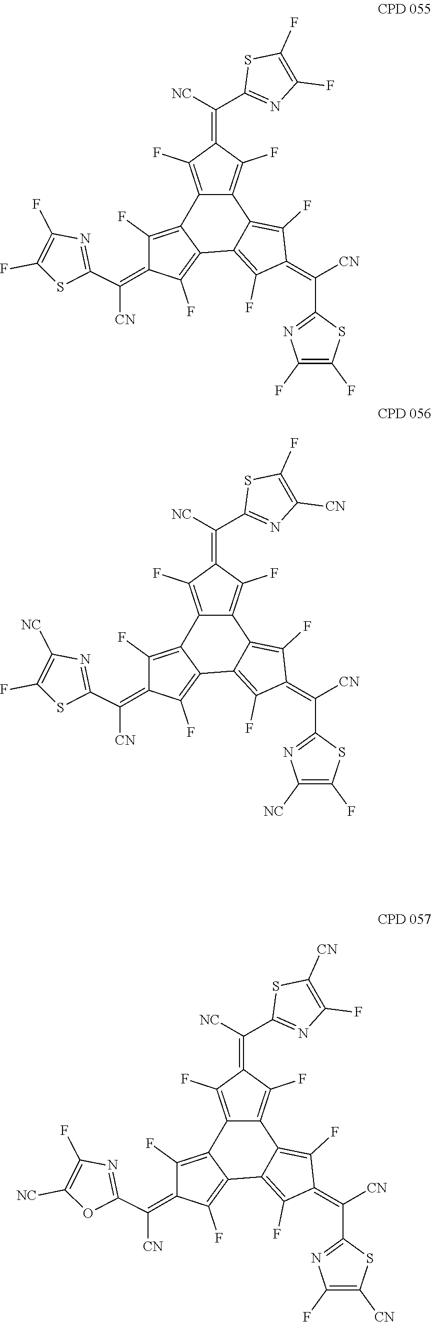 [SMILES: N#CC(=C1C(F)=c2c3c(c4c(c2=C1F)=C(F)C(=C(C#N)c1nc(F)c(F)s1)C=4F)=C(F)C(=C(C#N)c1nc(F)c(F)s1)C=3F)c1nc(F)c(F)s1 ; N#CC(=C1C(F)=c2c3c(c4c(c2=C1F)=C(F)C(=C(C#N)c1nc(C#N)c(F)s1)C=4F)=C(F)C(=C(C#N)c1nc(C#N)c(F)s1)C=3F)c1nc(C#N)c(F)s1 ; N#C/C(=C1/C(F)=c2c(c3c(c4c2=C(F)/C(=C(\C#N)c2nc(F)c(C#N)s2)C=4F)=C(F)/C(=C(\C#N)c2nc(F)c(C#N)s2)C=3F)=C1F)c1nc(F)c(C#N)o1]

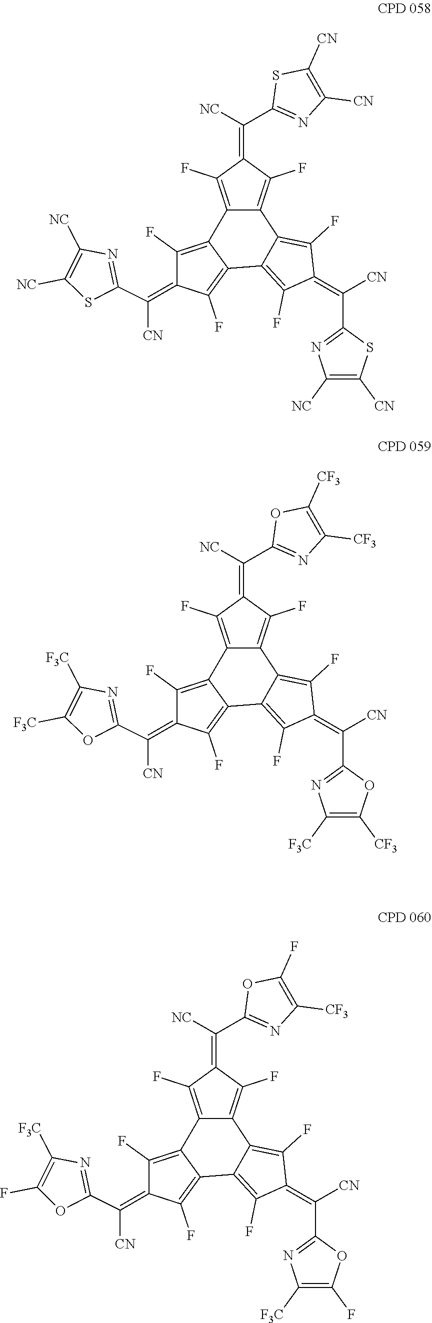 [SMILES: N#CC(=C1C(F)=c2c3c(c4c(c2=C1F)=C(F)C(=C(C#N)c1nc(C#N)c(C#N)s1)C=4F)=C(F)C(=C(C#N)c1nc(C#N)c(C#N)s1)C=3F)c1nc(C#N)c(C#N)s1 ; N#CC(=C1C(F)=c2c3c(c4c(c2=C1F)=C(F)C(=C(C#N)c1nc(C(F)(F)F)c(C(F)(F)F)o1)C=4F)=C(F)C(=C(C#N)c1nc(C(F)(F)F)c(C(F)(F)F)o1)C=3F)c1nc(C(F)(F)F)c(C(F)(F)F)o1 ; N#CC(=C1C(F)=c2c3c(c4c(c2=C1F)=C(F)C(=C(C#N)c1nc(C(F)(F)F)c(F)o1)C=4F)=C(F)C(=C(C#N)c1nc(C(F)(F)F)c(F)o1)C=3F)c1nc(C(F)(F)F)c(F)o1]

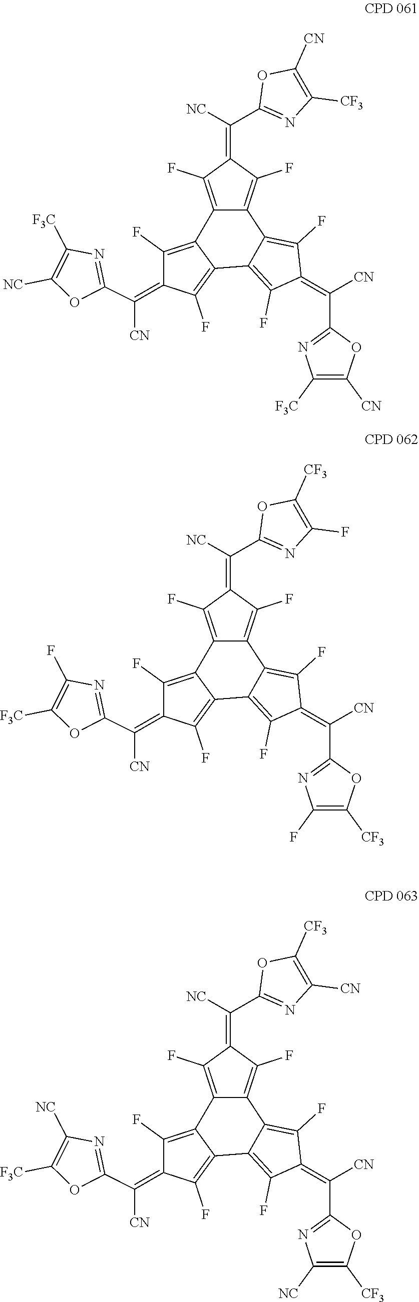 [SMILES: N#CC(=C1C(F)=c2c3c(c4c(c2=C1F)=C(F)C(=C(C#N)c1nc(C(F)(F)F)c(C#N)o1)C=4F)=C(F)C(=C(C#N)c1nc(C(F)(F)F)c(C#N)o1)C=3F)c1nc(C(F)(F)F)c(C#N)o1 ; N#CC(=C1C(F)=c2c3c(c4c(c2=C1F)=C(F)C(=C(C#N)c1nc(F)c(C(F)(F)F)o1)C=4F)=C(F)C(=C(C#N)c1nc(F)c(C(F)(F)F)o1)C=3F)c1nc(F)c(C(F)(F)F)o1 ; N#CC(=C1C(F)=c2c3c(c4c(c2=C1F)=C(F)C(=C(C#N)c1nc(C#N)c(C(F)(F)F)o1)C=4F)=C(F)C(=C(C#N)c1nc(C#N)c(C(F)(F)F)o1)C=3F)c1nc(C#N)c(C(F)(F)F)o1]

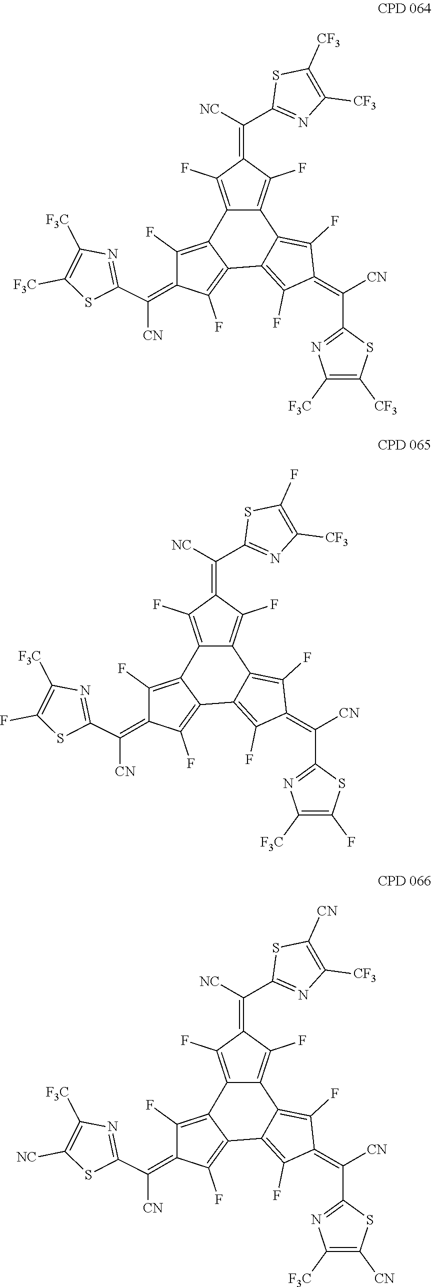 [SMILES: N#CC(=C1C(F)=c2c3c(c4c(c2=C1F)=C(F)C(=C(C#N)c1nc(C(F)(F)F)c(C(F)(F)F)s1)C=4F)=C(F)C(=C(C#N)c1nc(C(F)(F)F)c(C(F)(F)F)s1)C=3F)c1nc(C(F)(F)F)c(C(F)(F)F)s1 ; N#CC(=C1C(F)=c2c3c(c4c(c2=C1F)=C(F)C(=C(C#N)c1nc(C(F)(F)F)c(F)s1)C=4F)=C(F)C(=C(C#N)c1nc(C(F)(F)F)c(F)s1)C=3F)c1nc(C(F)(F)F)c(F)s1 ; N#CC(=C1C(F)=c2c3c(c4c(c2=C1F)=C(F)C(=C(C#N)c1nc(C(F)(F)F)c(C#N)s1)C=4F)=C(F)C(=C(C#N)c1nc(C(F)(F)F)c(C#N)s1)C=3F)c1nc(C(F)(F)F)c(C#N)s1]

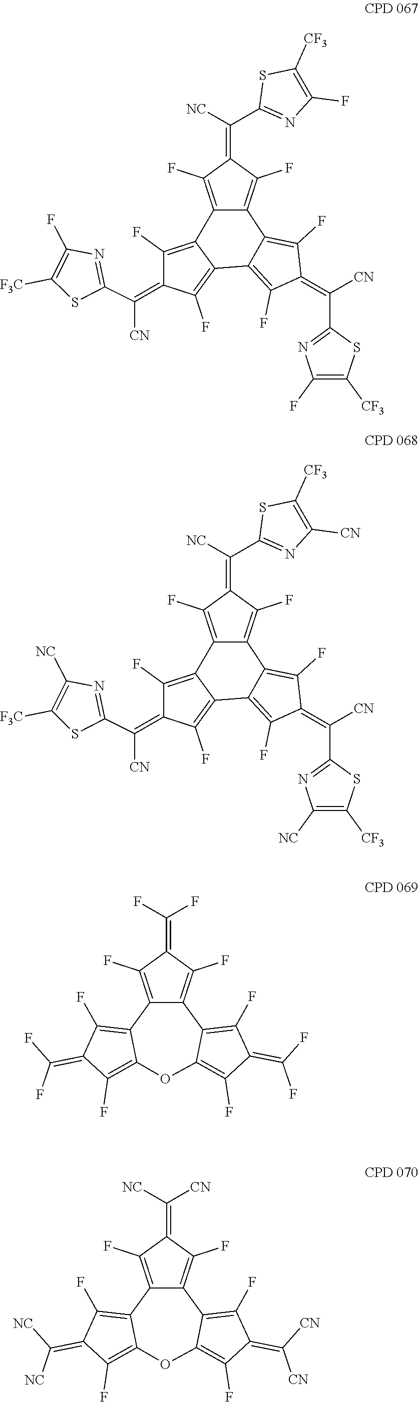 [SMILES: N#CC(=C1C(F)=c2c3c(c4c(c2=C1F)=C(F)C(=C(C#N)c1nc(F)c(C(F)(F)F)s1)C=4F)=C(F)C(=C(C#N)c1nc(F)c(C(F)(F)F)s1)C=3F)c1nc(F)c(C(F)(F)F)s1 ; N#CC(=C1C(F)=c2c3c(c4c(c2=C1F)=C(F)C(=C(C#N)c1nc(C#N)c(C(F)(F)F)s1)C=4F)=C(F)C(=C(C#N)c1nc(C#N)c(C(F)(F)F)s1)C=3F)c1nc(C#N)c(C(F)(F)F)s1 ; FC(F)=c1c(F)c2oc3c(F)c(=C(F)F)c(F)c3c3c(F)c(=C(F)F)c(F)c3c2c1F ; N#CC(C#N)=c1c(F)c2oc3c(F)c(=C(C#N)C#N)c(F)c3c3c(F)c(=C(C#N)C#N)c(F)c3c2c1F]

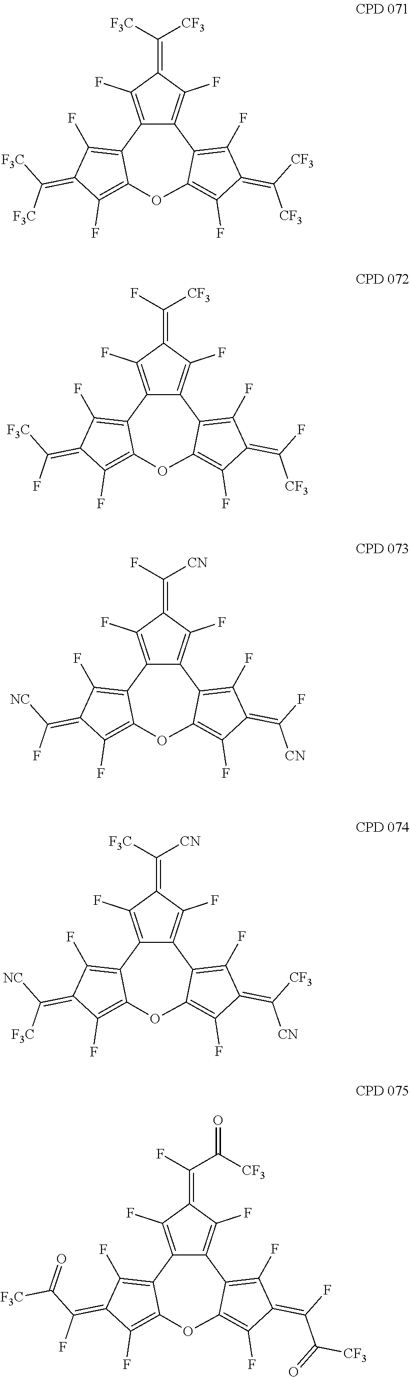 [SMILES: Fc1c(=C(C(F)(F)F)C(F)(F)F)c(F)c2c1oc1c(F)c(=C(C(F)(F)F)C(F)(F)F)c(F)c1c1c(F)c(=C(C(F)(F)F)C(F)(F)F)c(F)c21 ; F/C(=c1\c(F)c2oc3c(F)/c(=C(/F)C(F)(F)F)c(F)c3c3c(F)/c(=C(/F)C(F)(F)F)c(F)c3c2c1F)C(F)(F)F ; N#C/C(F)=c1/c(F)c2oc3c(F)/c(=C(/F)C#N)c(F)c3c3c(F)/c(=C(/F)C#N)c(F)c3c2c1F ; N#C/C(=c1/c(F)c2oc3c(F)/c(=C(\C#N)C(F)(F)F)c(F)c3c3c(F)/c(=C(\C#N)C(F)(F)F)c(F)c3c2c1F)C(F)(F)F ; O=C(/C(F)=c1/c(F)c2oc3c(F)/c(=C(/F)C(=O)C(F)(F)F)c(F)c3c3c(F)/c(=C(/F)C(=O)C(F)(F)F)c(F)c3c2c1F)C(F)(F)F]

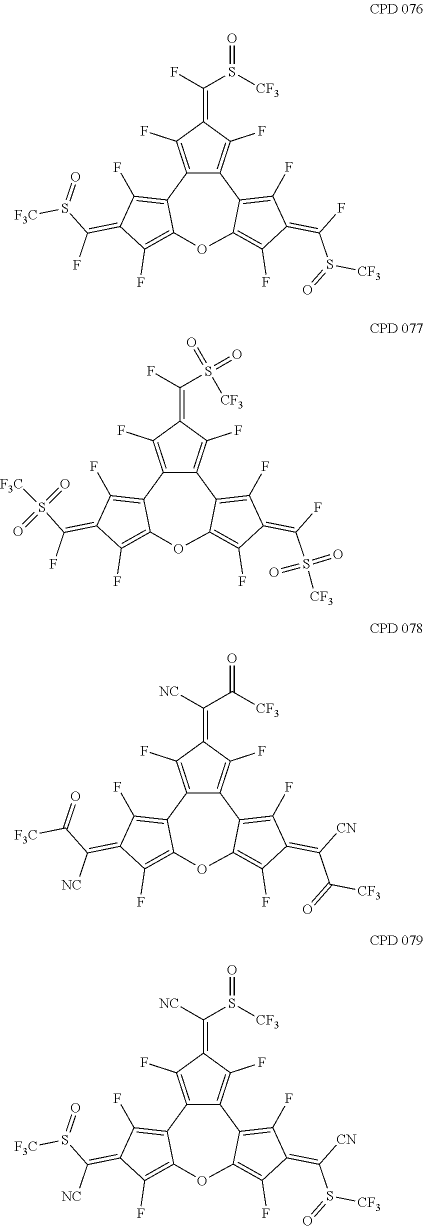 [SMILES: O=S(/C(F)=c1\c(F)c2oc3c(F)/c(=C(\F)S(=O)C(F)(F)F)c(F)c3c3c(F)/c(=C(\F)S(=O)C(F)(F)F)c(F)c3c2c1F)C(F)(F)F ; O=S(=O)(/C(F)=c1\c(F)c2oc3c(F)/c(=C(\F)S(=O)(=O)C(F)(F)F)c(F)c3c3c(F)/c(=C(\F)S(=O)(=O)C(F)(F)F)c(F)c3c2c1F)C(F)(F)F ; N#C/C(C(=O)C(F)(F)F)=c1/c(F)c2oc3c(F)/c(=C(\C#N)C(=O)C(F)(F)F)c(F)c3c3c(F)/c(=C(\C#N)C(=O)C(F)(F)F)c(F)c3c2c1F ; N#C/C(=c1/c(F)c2oc3c(F)/c(=C(\C#N)S(=O)C(F)(F)F)c(F)c3c3c(F)/c(=C(\C#N)S(=O)C(F)(F)F)c(F)c3c2c1F)S(=O)C(F)(F)F]

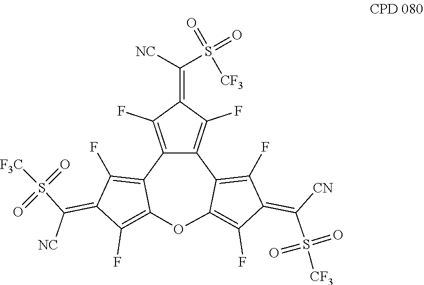 [SMILES: N#C/C(=c1/c(F)c2oc3c(F)/c(=C(\C#N)S(=O)(=O)C(F)(F)F)c(F)c3c3c(F)/c(=C(\C#N)S(=O)(=O)C(F)(F)F)c(F)c3c2c1F)S(=O)(=O)C(F)(F)F]

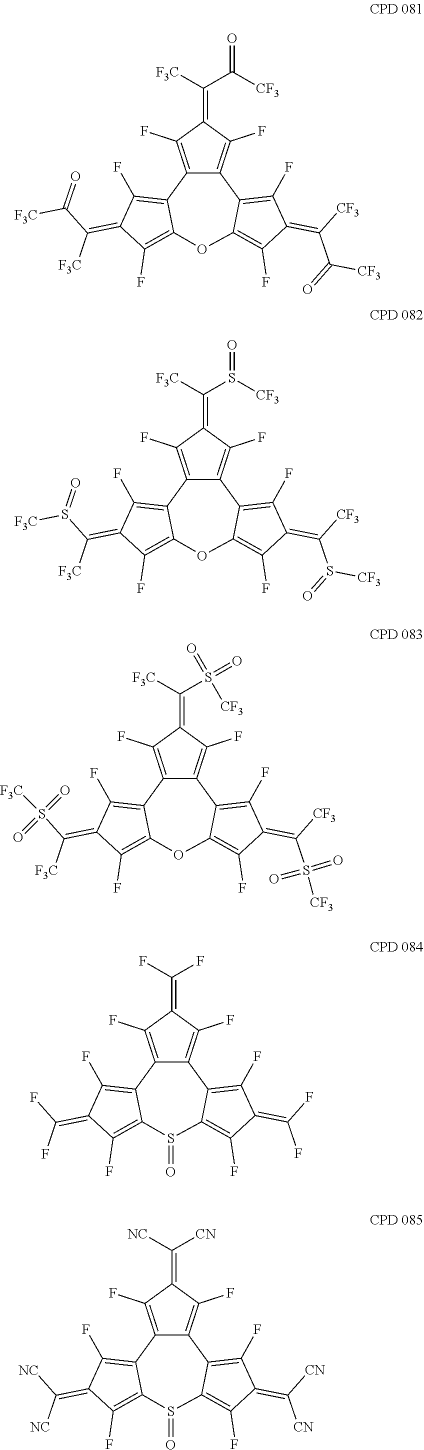

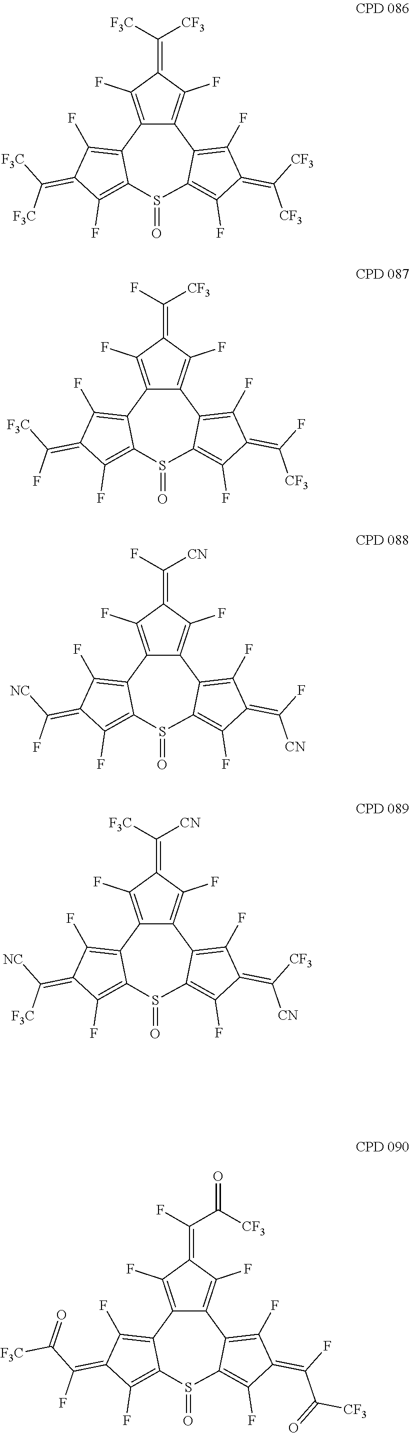

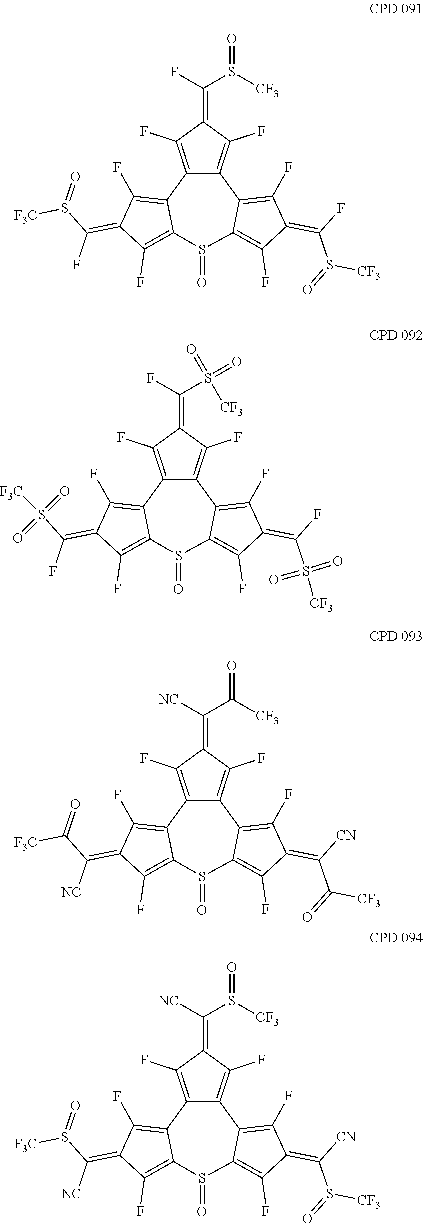 [SMILES: O=S1C2=C(F)/C(=C(/F)S(=O)C(F)(F)F)C(F)=C2C2=C(F)/C(=C(/F)S(=O)C(F)(F)F)C(F)=C2C2=C(F)/C(=C(/F)S(=O)C(F)(F)F)C(F)=C21 ; O=S1C2=C(F)/C(=C(/F)S(=O)(=O)C(F)(F)F)C(F)=C2C2=C(F)/C(=C(/F)S(=O)(=O)C(F)(F)F)C(F)=C2C2=C(F)/C(=C(/F)S(=O)(=O)C(F)(F)F)C(F)=C21 ; N#C/C(C(=O)C(F)(F)F)=C1/C(F)=C2C(=C1F)C1=C(F)/C(=C(/C#N)C(=O)C(F)(F)F)C(F)=C1S(=O)C1=C(F)/C(=C(/C#N)C(=O)C(F)(F)F)C(F)=C21 ; N#C/C(=C1/C(F)=C2C(=C1F)C1=C(F)/C(=C(/C#N)S(=O)C(F)(F)F)C(F)=C1S(=O)C1=C(F)/C(=C(/C#N)S(=O)C(F)(F)F)C(F)=C21)S(=O)C(F)(F)F]

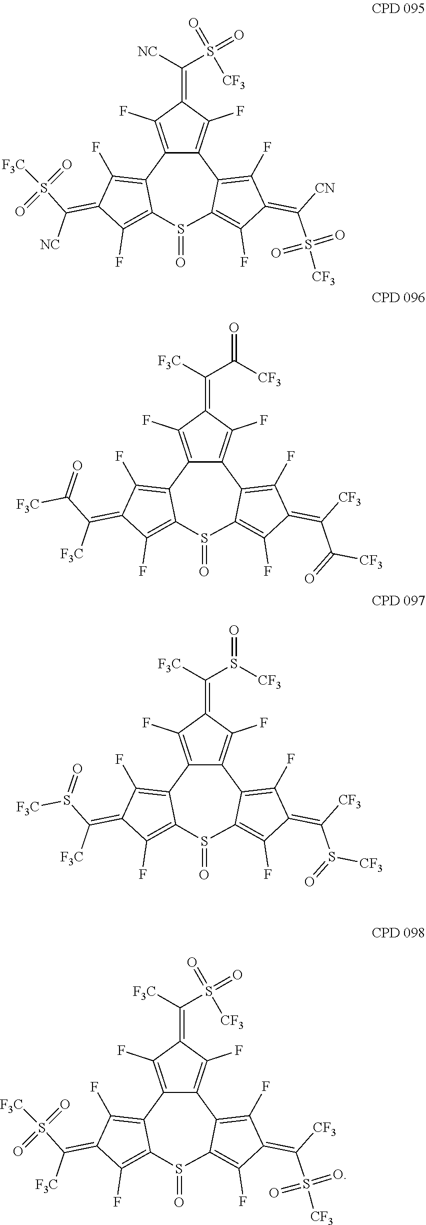

12. An electroluminescent device comprising a cathode, an anode, and an organic layer arranged between the cathode and the anode, wherein the organic layer comprises the organic compound according to claim 1.

13. The electroluminescent device according to claim 12, wherein the organic layer comprises a hole injection layer, and the hole injection layer comprises the organic compound as a hole injection material.

14. The electroluminescent device according to claim 13, wherein the hole injection layer further comprises at least one hole transport material containing triarylamine or carbazole as a matrix material.

15. The electroluminescent device according to claim 14, wherein an absolute value of a highest occupied molecular orbital energy level HOMO of the hole transport material containing triarylamine or carbazole is 4.8-6.8 eV.

16. An electroluminescent device comprising a cathode, an anode, and an organic layer arranged between the cathode and the anode, wherein the organic layer comprises the organic compound according to claim 2.

17. An electroluminescent device comprising a cathode, an anode, and an organic layer arranged between the cathode and the anode, wherein the organic layer comprises the organic compound according to claim 3.

18. An electroluminescent device comprising a cathode, an anode, and an organic layer arranged between the cathode and the anode, wherein the organic layer comprises the organic compound according to claim 4.

19. An electroluminescent device comprising a cathode, an anode, and an organic layer arranged between the cathode and the anode, wherein the organic layer comprises the organic compound according to claim 5.

20. An electroluminescent device comprising a cathode, an anode, and an organic layer arranged between the cathode and the anode, wherein the organic layer comprises the organic compound according to claim 11.
